SEQUENCE LISTING (38 sequences)

SEQ ID NO: 1 - protein, 288 aa, synthetic construct
MLGNGSAIVPIDQCFCLAWTDSLGSDTEQLVANILQWFAFGFSILILMFYAYQTWRATCGWEEVYVCCVELTKVIIEFFHEFDDPSMLYLANGHRVQWLRYAEWLLTCPVILIHLSNLTGLKDDYSKRTMRLLVSDVGTIVWGATSAMSTGYVKVIFFVLGCIYGANTFFHAAKVYIESYHVVPKGRPRTVVRIMAWLFFLSWGMFPVLFVVGPEGFDAISVYGSTIGHTIIDLMSKNCWGLLGHYLRVLIHQHIIIYGDIRKKTKINVAGEEMEVETMVDQEDEETV

SEQ ID NO: 2 - DNA, 864 bp, synthetic construct
  source 1..864: mol_type = other DNA
atgctgggaaacggcagcgccattgtgcctatcgaccagtgcttttgcctggcttggaccgacagcctgggaagcgatacagagcagctggtggccaacatcctccagtggttcgccttcggcttcagcatcctgatcctgatgttctacgcctaccagacttggagagccacttgcggttgggaggaggtctacgtctgttgcgtcgagctgaccaaggtcatcatcgagttcttccacgagttcgacgaccccagcatgctgtacctggctaacggacaccgagtccagtggctgagatacgcagagtggctgctgacttgtcccgtcatcctgatccacctgagcaacctgacaggcctgaaggacgactacagcaagcggaccatgaggctgctggtgtcagacgtgggaaccatcgtgtggggagctacaagcgccatgagcacaggctacgtcaaggtcatcttcttcgtgctgggttgcatctacggcgccaacaccttcttccacgccgccaaggtgtatatcgagagctaccacgtggtgccaaagggcagacctagaaccgtcgtgcggatcatggcttggctgttcttcctgtcttggggcatgttccccgtgctgttcgtcgtgggaccagaaggattcgacgccatcagcgtgtacggctctaccattggccacaccatcatcgacctcatgagcaagaattgttggggcctgctgggacactatctgagagtgctgatccaccagcacatcatcatctacggcgacatccgcaagaagaccaagatcaacgtggccggcgaggagatggaagtggagaccatggtggaccaggaggacgaggagacagtg

SEQ ID NO: 3 - protein, 288 aa, synthetic construct
MLGNGSAIVPIDQCFCLAWTDSLGSDTEQLVANILQWFAFGFSILILMFYAYQTWRATCGWEEVYVCCVELTKVIIEFFHEFDDPSMLYLANGHRVQWLRYAEWLLTCPVICIHLSNLTGLKDDYSKRTMRLLVSDVGTIVWGATSAMSTGYVKVIFFVLGCIYGANTFFHAAKVYIESYHVVPKGRPRTVVRIMAWLFFLSWGMFPVLFVVGPEGFDAISVYGSTIGHTIIDLMSKNCWGLLGHYLRVLIHQHIIIYGDIRKKTKINVAGEEMEVETMVDQEDEETV

SEQ ID NO: 4 - protein, 288 aa, synthetic construct
MLGNGSAIVPIDQCFCLAWTDSLGSDTEQLVANILQWFAFGFSILILMFYAYQTWRATCGWEEVYVCCVELTKVIIEFFHEFDDPSMLYLANGHRVQWLRYAEWLLTCPVICIHLSNLTGLKDDYSKRTMRLLVSDVGCIVWGATSAMSTGYVKVIFFVLGCIYGANTFFHAAKVYIESYHVVPKGRPRTVVRIMAWLFFLSWGMFPVLFVVGPEGFDAISVYGSTIGHTIIDLMSKNCWGLLGHYLRVLIHQHIIIYGDIRKKTKINVAGEEMEVETMVDQEDEETV

SEQ ID NO: 5 - protein, 288 aa, synthetic construct
MLGNGSAIVPIDQCFCLAWTDSLGSDTEQLVANILQWFAFGFSILILMFYAYQTWRATCGWEEVYVCCVELTKVIIEFFHEFDDPSMLYLANGERVQWLRYAEWLLTCPVICIHLSNLTGLKDDYSKRTMRLLVSDVGTIVWGATSAMSTGYVKVIFFVLGCIYGANTFFHAAKVYIESYHVVPKGRPRTVVRIMAWLFFLSWGMFPVLFVVGPEGFDAISVYGSTIGHTIIDLMSKNCWGLLGHYLRVLIHQHIIIYGDIRKTTKINVAGEEMEVETMVDQEDEETV

SEQ ID NO: 6 - protein, 728 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSANNNSEYSWTGATKYHLNGRDSLVNPGPAMASHKDDEEKFFPQSGVLIFGKQGSEKTNVDIEKVMITDEEEIRTTNPVATEQYGSVSTNLQRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWEQKENSKRWNPEIQYTSNYNKSVNVDFTVDTNGVYSEPRPIGTRYLTRNL

SEQ ID NO: 7 - protein, 543 aa, synthetic construct
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 7 aa, synthetic construct
LGETTRP

SEQ ID NO: 9 - protein, 10 aa, synthetic construct
LAISDQTKHA

SEQ ID NO: 10 - DNA, 1709 bp, synthetic construct
  source 1..1709: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 11 - DNA, 1784 bp, synthetic construct
  source 1..1784: mol_type = other DNA
caagcaggaggctgctgtgtgctgggagctgtcaggctcgtcctgaacagggaagggcccatccacctcccaaacccagtttatgcagtccttcgcaatgtcaggctcagggcctggcaccagccaagctccccacccttcccactgttaaaatggataggagcagggctaggcccagcctgttgactctgggcttccaccaggagaagtggttctggcagtagaaactatcggggcctgggagaggcgggggaagagagaaaggtggcatgtttcttgcttgctccctctaccagccttgtccaaatccccgcagccaccctaatccagcctgtctaatggagcccaagccggctcaggccctcggacgaggagcctgctaatccctgtggctaggagctcaccacctgtctccaggacgccctttgctctcttggcatcagagagccaaatcctgggcctcggatggggggatgataaaagcatcttttggccaagccccctcaccttggcctccacgatgagatggggagttaggtgcagagagcgttggcacagtgagcaccgcagctcgagtggctgcctcagacccagagcccgaggagactttatacggagccagaacgaccccgcggggttccatcctcccaagcaataggcgggagtgggagctgcgaggaaagccggcccctcccctccctccatccaaggcagtgtgggctgtttgtttcatgccattctgggtgtgaatcctgatgcccacacatgccagctgcatgcacttgggcaactcaactcactcctcgagggctgtttctcgactgcagggtgttgtaagttcgctaatactaaaggcttctccctcctggccccttcctgcccctcgctcttcctcctcttccttaggccctcccagctcaggcagcccctgccccctgcagggttctgcaaggagaaagctggggaataccttaggcaactgcagtcaggagcactggtggccaggacagagacagagagacagaaaaggggtcagggacagagagagataaccgcagggagagacaggaagggacagagacagaaaagatttccaagaagaggacagaggcagaaagccagggacagagactgagaaacagagacctagaggcagaagaagactgagatagagatggacagagattgtgtcagacacagccccagagacagccagacagtctgagtcagacgcaaaccaaagacaagaaaacaggaaaacagacccagagattgggagagggaggggaaggagatgcggggagagccagcaccgccaccccccacactcaggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccg

SEQ ID NO: 12 - DNA, 547 bp, synthetic construct
  source 1..547: mol_type = other DNA
ccaaagacaagaaaacaggaaaacagacccagagattgggagagggaggggaaggagatgcggggagagccagcaccgccaccccccacactcaggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccg

SEQ ID NO: 13 - DNA, 198 bp, synthetic construct
  source 1..198: mol_type = other DNA
gatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgta

SEQ ID NO: 14 - DNA, 601 bp, synthetic construct
  source 1..601: mol_type = other DNA
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcgg

SEQ ID NO: 15 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 864 bp, synthetic construct
  source 1..864: mol_type = other DNA
atgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtg

SEQ ID NO: 18 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 19 - DNA, 9349 bp, synthetic construct
  source 1..9349: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtggaattcggaggcggaggtggagctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactaataactcgagggtaaccgtggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgtaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgatatcgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 20 - DNA, 8761 bp, synthetic construct
  source 1..8761: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtggaattcggaggcggaggtggagctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactaataactcgagggtaaccgtggtaccgatctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgtaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgatatcgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 21 - DNA, 8615 bp, synthetic construct
  source 1..8615: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtgtaataactcgagggtaaccgtggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgtaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgatatcgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 22 - DNA, 9462 bp, synthetic construct
  source 1..9462: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtggaattcgtagcagtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaataactcgagggtaaccgtggtaccgatctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgtaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgatatcgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 23 - DNA, 8025 bp, synthetic construct
  source 1..8025: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatgcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtgtaataactcgagggtaaccgtggtaccgatctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgtaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgatatcgcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 24 - DNA, 500 bp, synthetic construct
  source 1..500: mol_type = other DNA
ttaaaggcagtctaggggagaagcagacccagggagtcagagaggcagagagagaagagagcccttcctccactctcaagctctggagggggtctctgccctcaccctcatccctccccagaatccttaaatcctctagactgtagctctgattttacagctgtcacagactcgtcctactagccagaggttggctcaggtaagcaccactggggaggtagcctagggtgcgctggggtgggtccagaggaagagctgcccagaactgtgggggaaggagcgggaccgaccatcaacagggggacttttcagggagaatgagagcaatcctctggaggcctgggagaggctgctgagttgctggtgcgcgagtcaccaacttttcctgcgctctcggtgtccggccagaatcccgaagtggcagctgagcacggggtggcagcttcgtccgccggctctcaaggcgtcccggtaacttcctttcccgcagtccaggagca

SEQ ID NO: 25 - DNA, 201 bp, synthetic construct
  source 1..201: mol_type = other DNA
gatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgt

SEQ ID NO: 26 - DNA, 690 bp, synthetic construct
  source 1..690: mol_type = other DNA
ggtgagtcccccaccccactcatcctccctgatgcttcctgtgtgggatgctcatttccacatttgtctcggagtcccacatgctgagtaactctgagatttgctttaaaatgccatgcaggtaatttaaatgggaaggtctgatccaagtgatgaagtgcagccttgatagcatgcttcctccgccctcccacaggcttccatcttttgtggggtgcccacctccacaccttttcttttagctagagtggtcaagtggacaagctggtcattagcaatcaaggcgtttcagatctggaagtgggtggtgccattatggatcagtgagccctgtattttttgtgcctctgcacaaggtgggtagtgaagccctgtccattacataaccatggcatcccctagccatgacataaagggcagtgaaaaattctttaaggatgccagagctgctttttccatttgtgtgtatgcgtgcaggtgtgtgttgtacatgacacaagtgtatgtgtgtgcatgtggaggcctgaggttgatttcaggaatcatcctcaattctttttctaccttattcactgaggcagggtctgtggagagatcaccgatatggctactgtgggattcccctgtctctgccttcagagccactcctggatacacagtacacctggctcagatggtcaccaccctcct

SEQ ID NO: 27 - DNA, 800 bp, synthetic construct
  source 1..800: mol_type = other DNA
actccaggccatgagcaactcctcacatctccctaagcccttcctgtcgccctctggagtcttttgttctgggaatgagacaggcttgactggctgaaggttctccgggcctggcctgggaaacacaggaaaacacgactatttttattgttcattgtgggagagagaactggtaggcaaacccaagagcagaaaatgtaccgtgagggacactgccccagtaaaccctgaaacctacattatcctaagccagccaaggttcttttccagcctgggaagttgagcgtgacattggtggctgaatttgtagacagaatggcttctgagtgcccctgacattccccaaaaggaggctctctgcattaatccatttgtctttattataataaaatatccaagtcagggcgttttttaaggaaaagacttatttttaacatcaactcttggaggtgaaagttcaggcagcgtgacaccagctctgctgaggacctagcttgcatcacattttgacaaatgttatggaaagagggagtagagaaggaaagagtgcatggagagaaggaacatcagaaagaagagggacagggttcactctttgatagctattcaccttcacagaattacctcacccttccagaggtcaagagcaacatccccagtgacccaataaccttgcactaagccacacctcttttttattttttatgagacagggcctcactctgtatccctgaacttgctatgtggaccaagctgtcctcttgagtgctgggattaaaggcattcagtatcagggctgg

SEQ ID NO: 28 - DNA, 2226 bp, synthetic construct
  source 1..2226: mol_type = other DNA
ggtgagtcccccaccccactcatcctccctgatgcttcctgtgtgggatgctcatttccacatttgtctcggagtcccacatgctgagtaactctgagatttgctttaaaatgccatgcaggtaatttaaatgggaaggtctgatccaagtgatgaagtgcagccttgatagcatgcttcctccgccctcccacaggcttccatcttttgtggggtgcccacctccacaccttttcttttagctagagtggtcaagtggacaagctggtcattagcaatcaaggcgtttcagatctggaagtgggtggtgccattatggatcagtgagccctgtattttttgtgcctctgcacaaggtgggtagtgaagccctgtccattacataaccatggcatcccctagccatgacataaagggcagtgaaaaattctttaaggatgccagagctgctttttccatttgtgtgtatgcgtgcaggtgtgtgttgtacatgacacaagtgtatgtgtgtgcatgtggaggcctgaggttgatttcaggaatcatcctcaattctttttctaccttattcactgaggcagggtctgtggagagatcaccgatatggctactgtgggattcccctgtctctgccttcagagccactcctggatacacagtacacctggctcagatggtcaccaccctcctagcgctcactccaggccatgagcaactcctcacatctccctaagcccttcctgtcgccctctggagtcttttgttctgggaatgagacaggcttgactggctgaaggttctccgggcctggcctgggaaacacaggaaaacacgactatttttattgttcattgtgggagagagaactggtaggcaaacccaagagcagaaaatgtaccgtgagggacactgccccagtaaaccctgaaacctacattatcctaagccagccaaggttcttttccagcctgggaagttgagcgtgacattggtggctgaatttgtagacagaatggcttctgagtgcccctgacattccccaaaaggaggctctctgcattaatccatttgtctttattataataaaatatccaagtcagggcgttttttaaggaaaagacttatttttaacatcaactcttggaggtgaaagttcaggcagcgtgacaccagctctgctgaggacctagcttgcatcacattttgacaaatgttatggaaagagggagtagagaaggaaagagtgcatggagagaaggaacatcagaaagaagagggacagggttcactctttgatagctattcaccttcacagaattacctcacccttccagaggtcaagagcaacatccccagtgacccaataaccttgcactaagccacacctcttttttattttttatgagacagggcctcactctgtatccctgaacttgctatgtggaccaagctgtcctcttgagtgctgggattaaaggcattcagtatcagggctggtttataatccggagatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgtgaccggtatttaaatttaaaggcagtctaggggagaagcagacccagggagtcagagaggcagagagagaagagagcccttcctccactctcaagctctggagggggtctctgccctcaccctcatccctccccagaatccttaaatcctctagactgtagctctgattttacagctgtcacagactcgtcctactagccagaggttggctcaggtaagcaccactggggaggtagcctagggtgcgctggggtgggtccagaggaagagctgcccagaactgtgggggaaggagcgggaccgaccatcaacagggggacttttcagggagaatgagagcaatcctctggaggcctgggagaggctgctgagttgctggtgcgcgagtcaccaacttttcctgcgctctcggtgtccggccagaatcccgaagtggcagctgagcacggggtggcagcttcgtccgccggctctcaaggcgtcccggtaacttcctttcccgcagtccaggagca

SEQ ID NO: 29 - DNA, 4053 bp, synthetic construct
  source 1..4053: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcaggcctcctagggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtgtaataactcgagggtaaccgtggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggcctcgaggaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - DNA, 4240 bp, synthetic construct
  source 1..4240: mol_type = other DNA
ggtgagtcccccaccccactcatcctccctgatgcttcctgtgtgggatgctcatttccacatttgtctcggagtcccacatgctgagtaactctgagatttgctttaaaatgccatgcaggtaatttaaatgggaaggtctgatccaagtgatgaagtgcagccttgatagcatgcttcctccgccctcccacaggcttccatcttttgtggggtgcccacctccacaccttttcttttagctagagtggtcaagtggacaagctggtcattagcaatcaaggcgtttcagatctggaagtgggtggtgccattatggatcagtgagccctgtattttttgtgcctctgcacaaggtgggtagtgaagccctgtccattacataaccatggcatcccctagccatgacataaagggcagtgaaaaattctttaaggatgccagagctgctttttccatttgtgtgtatgcgtgcaggtgtgtgttgtacatgacacaagtgtatgtgtgtgcatgtggaggcctgaggttgatttcaggaatcatcctcaattctttttctaccttattcactgaggcagggtctgtggagagatcaccgatatggctactgtgggattcccctgtctctgccttcagagccactcctggatacacagtacacctggctcagatggtcaccaccctcctagcgctcactccaggccatgagcaactcctcacatctccctaagcccttcctgtcgccctctggagtcttttgttctgggaatgagacaggcttgactggctgaaggttctccgggcctggcctgggaaacacaggaaaacacgactatttttattgttcattgtgggagagagaactggtaggcaaacccaagagcagaaaatgtaccgtgagggacactgccccagtaaaccctgaaacctacattatcctaagccagccaaggttcttttccagcctgggaagttgagcgtgacattggtggctgaatttgtagacagaatggcttctgagtgcccctgacattccccaaaaggaggctctctgcattaatccatttgtctttattataataaaatatccaagtcagggcgttttttaaggaaaagacttatttttaacatcaactcttggaggtgaaagttcaggcagcgtgacaccagctctgctgaggacctagcttgcatcacattttgacaaatgttatggaaagagggagtagagaaggaaagagtgcatggagagaaggaacatcagaaagaagagggacagggttcactctttgatagctattcaccttcacagaattacctcacccttccagaggtcaagagcaacatccccagtgacccaataaccttgcactaagccacacctcttttttattttttatgagacagggcctcactctgtatccctgaacttgctatgtggaccaagctgtcctcttgagtgctgggattaaaggcattcagtatcagggctggtttataatccggagatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgtgaccggtatttaaatttaaaggcagtctaggggagaagcagacccagggagtcagagaggcagagagagaagagagcccttcctccactctcaagctctggagggggtctctgccctcaccctcatccctccccagaatccttaaatcctctagactgtagctctgattttacagctgtcacagactcgtcctactagccagaggttggctcaggtaagcaccactggggaggtagcctagggtgcgctggggtgggtccagaggaagagctgcccagaactgtgggggaaggagcgggaccgaccatcaacagggggacttttcagggagaatgagagcaatcctctggaggcctgggagaggctgctgagttgctggtgcgcgagtcaccaacttttcctgcgctctcggtgtccggccagaatcccgaagtggcagctgagcacggggtggcagcttcgtccgccggctctcaaggcgtcccggtaacttcctttcccgcagtccaggagcagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggactctacttgagggtgctgatccaccagcacatcatcatctacggcgacatcagaaagaccaccaaaatcaacgtggccggagaagaaatggaagtcgagacaatggtggaccaagaggacgaggaaacagtgtaataactcgagggtaaccgtggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 31 - DNA, 4574 bp, synthetic construct
  source 1..4574: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcaggcctcctagggccgcacgcgtacgcgtggtgagtcccccaccccactcatcctccctgatgcttcctgtgtgggatgctcatttccacatttgtctcggagtcccacatgctgagtaactctgagatttgctttaaaatgccatgcaggtaatttaaatgggaaggtctgatccaagtgatgaagtgcagccttgatagcatgcttcctccgccctcccacaggcttccatcttttgtggggtgcccacctccacaccttttcttttagctagagtggtcaagtggacaagctggtcattagcaatcaaggcgtttcagatctggaagtgggtggtgccattatggatcagtgagccctgtattttttgtgcctctgcacaaggtgggtagtgaagccctgtccattacataaccatggcatcccctagccatgacataaagggcagtgaaaaattctttaaggatgccagagctgctttttccatttgtgtgtatgcgtgcaggtgtgtgttgtacatgacacaagtgtatgtgtgtgcatgtggaggcctgaggttgatttcaggaatcatcctcaattctttttctaccttattcactgaggcagggtctgtggagagatcaccgatatggctactgtgggattcccctgtctctgccttcagagccactcctggatacacagtacacctggctcagatggtcaccaccctcctagcgctcactccaggccatgagcaactcctcacatctccctaagcccttcctgtcgccctctggagtcttttgttctgggaatgagacaggcttgactggctgaaggttctccgggcctggcctgggaaacacaggaaaacacgactatttttattgttcattgtgggagagagaactggtaggcaaacccaagagcagaaaatgtaccgtgagggacactgccccagtaaaccctgaaacctacattatcctaagccagccaaggttcttttccagcctgggaagttgagcgtgacattggtggctgaatttgtagacagaatggcttctgagtgcccctgacattccccaaaaggaggctctctgcattaatccatttgtctttattataataaaatatccaagtcagggcgttttttaaggaaaagacttatttttaacatcaactcttggaggtgaaagttcaggcagcgtgacaccagctctgctgaggacctagcttgcatcacattttgacaaatgttatggaaagagggagtagagaaggaaagagtgcatggagagaaggaacatcagaaagaagagggacagggttcactctttgatagctattcaccttcacagaattacctcacccttccagaggtcaagagcaacatccccagtgacccaataaccttgcactaagccacacctcttttttattttttatgagacagggcctcactctgtatccctgaacttgctatgtggaccaagctgtcctcttgagtgctgggattaaaggcattcagtatcagggctggtttataatccggagatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgtgaccggtatttaaatttaaaggcagtctaggggagaagcagacccagggagtcagagaggcagagagagaagagagcccttcctccactctcaagctctggagggggtctctgccctcaccctcatccctccccagaatccttaaatcctctagactgtagctctgattttacagctgtcacagactcgtcctactagccagaggttggctcaggtaagcaccactggggaggtagcctagggtgcgctggggtgggtccagaggaagagctgcccagaactgtgggggaaggagcgggaccgaccatcaacagggggacttttcagggagaatgagagcaatcctctggaggcctgggagaggctgctgagttgctggtgcgcgagtcaccaacttttcctgcgctctcggtgtccggccagaatcccgaagtggcagctgagcacggggtggcagcttcgtccgccggctctcaaggcgtcccggtaacttcctttcccgcagtccaggagcagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggactctacttgagggtgctgatccaccagcacatcatcatctacggcgacatcagaaagaccaccaaaatcaacgtggccggagaagaaatggaagtcgagacaatggtggaccaagaggacgaggaaacagtgtaataactcgagggtaaccgtggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggcctcgaggaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 32 - DNA, 4458 bp, synthetic construct
  source 1..4458: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtggaattcggaggcggaggtggagctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactaataactcgagggtaaccgtggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 33 - DNA, 3869 bp, synthetic construct
  source 1..3869: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtggaattcggaggcggaggtggagctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaactaataactcgagggtaaccgtggtaccgatctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 34 - DNA, 3723 bp, synthetic construct
  source 1..3723: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtgtaataactcgagggtaaccgtggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 35 - DNA, 4571 bp, synthetic construct
  source 1..4571: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtggaattcgtagcagtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaataactcgagggtaaccgtggtaccgatctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 36 - DNA, 3134 bp, synthetic construct
  source 1..3134: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagagctcgcgatccgccaccatgctgggcaacggatctgctatcgttcctatcgaccagtgcttctgcctggcttggaccgactctctgggctctgataccgaacagctggtggccaatatcctgcagtggttcgccttcggcttcagcattctgatcctgatgttctacgcctaccagacctggcgggccacctgtggctgggaggaagtgtatgtgtgctgcgtggaactgacaaaggtgatcatcgagttcttccacgagttcgacgaccccagcatgctgtacctggccaatggcgagagagtgcagtggctgcggtacgccgagtggctgctgacctgtcctgtcatctgcatccacctgtccaacctgaccggcctgaaggacgattacagcaaaagaaccatgagactgctcgtgtccgatgtgggcaccatcgtgtggggcgctaccagcgctatgagcaccggctacgtgaaggttatcttctttgtgctgggttgcatctatggcgccaacactttcttccacgccgccaaggtgtacatcgagagctaccacgtggtccccaagggcagacctagaacagtggtgcggatcatggcctggctgtttttcctgagctggggaatgtttccagtgctgttcgtggtgggccctgagggatttgatgccatcagcgtgtacggcagcacaatcggccatacaatcattgacctgatgagcaagaactgttggggcctgctgggacactacttgagggtgctgatccaccagcacatcatcatctacggcgacatcagaaagaccaccaaaatcaacgtggccggagaagaaatggaagtcgagacaatggtggaccaagaggacgaggaaacagtgtaataactcgagggtaaccgtggtaccgatctgatgcggggatccggtaacgtcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 37 - protein, 735 aa, synthetic construct [sequence omitted]

SEQ ID NO: 38 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

(19) United States
(12) Patent Application Publication
(10) Pub. No.: US 2025/0387513 A1
(43) Pub. Date: Dec. 25, 2025

(54) GENE THERAPY COMPOSITIONS AND METHODS FOR TREATING DISEASES OF THE RETINA

(71) Applicants: Ray Therapeutics, Inc., Berkeley, CA (US); Wayne State University, Detroit, MI (US)

(72) Inventors: Peter Francis, Portland, OR (US); Zhuo-Hua Pan, Troy, MI (US); Paul Bresge, Berkeley, CA (US); Gary Abrams, Detroit, MI (US); Qi Lu, Detroit, MI (US)

(73) Assignees: Ray Therapeutics, Inc., Berkeley, CA (US); Wayne State University, Detroit, MI (US)

(21) Appl. No.: 19/300,897

(22) Filed: Aug. 15, 2025

Related U.S. Application Data
(63) Continuation of application No. PCT/US2024/016073, filed on Feb. 16, 2024.
(60) Provisional application No. 63/485,596, filed on Feb. 17, 2023.

(51) Int. Cl. A61K 48/00; A61K 9/00; A61K 38/00; A61P 27/02; C07K 14/005; C07K 14/405; C12N 15/86

(52) U.S. Cl. CPC A61K 48/005; A61K 9/0048; A61K 48/0075; A61K 48/0083; A61P 27/02; C07K 14/005; C07K 14/405; C12N 15/86; A61K 38/00; C12N 2750/14122; C12N 2750/14143; C12N 2830/48; C12N 2830/50

(57) ABSTRACT

The present invention provides recombinant AAV particles comprising an opsin transgene for therapy of retinal diseases, and related compositions and methods.

### CROSS-REFERENCE TO RELATED APPLICATIONS

This application is a continuation application claiming the benefit of and priority to International Patent Application No. PCT/US2024/016073, entitled “GENE THERAPY COMPOSITIONS AND METHODS FOR TREATING DISEASES OF THE RETINA”, filed Feb. 16, 2024, which claims the benefit of and priority to U.S. provisional application No. 63/485,596, filed Feb. 17, 2023, the contents of each of which are hereby incorporated by reference in their entirety.

### FIELD OF THE INVENTION

The present invention relates to therapeutic compositions and methods for treating diseases of the retina by expression of a heterologous gene using an AAV delivery system.

### STATEMENT REGARDING A SEQUENCE LISTING

The present application contains a sequence listing entitled 60650501_Sequence Listing.xml created Aug. 14, 2025 and is 126242 bytes in size. The sequence listing is submitted electronically along with the filing of the present application and is hereby incorporated by reference in its entirety.

### BACKGROUND

Gene therapy represents a promising approach for improving and restoring vision in humans and there are a number of clinical trials relating to gene therapies for the treatment of retinal diseases. Adeno-associated virus (AAV)-based systems for delivery of a therapeutic transgene are utilized by some clinical candidates, in part because the safety profile of AAV has been well-characterized. For example, Luxturna™ (SparkTherapeutics) is an AAV2 vector encoding retinal pigment epithelium-specific protein 65-kD (RPE65) that was approved in 2017 for patients having biallelic RPE65 mutation-associated retinal dystrophy. RPE65 resides in retinal pigment epithelium (RPE) that is responsible for regeneration of 11-cis retinol in the visual cycle.

However, significant challenges remain for successful translation of any particular therapy to the clinic, including efficient targeting and expression of therapeutic transgenes in retinal cells. Although much is known about the components required for AAV vector design, it is not possible to know how a particular vector design will function based on prior results with individual vector elements. Instead, it remains unpredictable how a particular combination of elements will function for delivery and expression of a particular transgene, particularly in humans. There is a need for additional AAV vector-transgene delivery systems for human use in treating disease of the retina. The present invention addresses this need.

### BRIEF SUMMARY

The present invention provides AAV vectors and related compositions and methods for the expression of therapeutic transgenes in the mammalian and primate eye, in particular the expression of modified channelrhodopsins for improving visual acuity and/or restoring vision in a subject in need thereof. Accordingly, the invention provides infectious recombinant adeno-associated virus (rAAV) particles, related vector constructs, related compositions, including pharmaceutical compositions, and related methods including methods for gene therapy of retinal diseases and disorders.

In one aspect, an infectious recombinant adeno-associated virus (rAAV) particle includes (i) a capsid protein having a modified amino acid sequence relative to a native AAV capsid of serotype 2 and (ii) a vector genome consisting of a heterologous polynucleotide that includes from 5′ to 3′ (a) an AAV2 inverted terminal repeat sequence (ITR1); (b) a promoter sequence; (c) a polynucleotide sequence encoding a channelrhodopsin; (d) a polyadenylation sequence; and (e) an AAV2 inverted terminal repeat sequence (ITR2), where the heterologous polynucleotide does not encode a fluorescent protein or other reporter protein. The rAAV particle may also include where the heterologous polynucleotide further includes a woodchuck hepatitis virus posttranscriptional regulatory element (WPRE) between the polyadenylation sequence and the ITR2 sequence. The rAAV particle may also include where the promoter is selected from a cytomegalovirus (CMV) promoter, an elongation factor 1a (EF1a) promoter, a simian virus 40 (SV40) promoter, a chicken beta-actin promoter, an mGluR6 promoter, and a CAG promoter. The rAAV particle may also include where the heterologous polynucleotide further includes an enhancer sequence. The rAAV particle may also include where the channelrhodopsin includes the amino acid sequence of Chrown (SEQ ID NO: 5). The rAAV particle may also include where the channelrhodopsin includes the amino acid sequence of Chrown (SEQ ID NO: 5), or an amino acid sequence at least 80%, at least 85%, at least 90%, at least 95%, at least 97%, at least 98%, or at least 99% identical thereto. The rAAV particle may also include where the polyadenylation signal is a human growth hormone polyadenylation sequence (hGHpA) or a Simian virus 40 polyadenylation sequence. The rAAV particle may also include where the heterologous polynucleotide includes pCAG-Chrown-mWPRE-hGHpA (SEQ ID NO: 21) or pCAG-Chrown-hGHpA (SEQ ID NO: 23). The rAAV particle may also include where the promoter is a CAG promoter. The rAAV particle may also include where the CAG promoter includes SEQ ID NO: 10 and the WPRE element includes SEQ ID NO: 14. The rAAV particle may also include where the enhancer is a CMV enhancer or an mGluR6 enhancer. The rAAV particle may also include where the polyadenylation signal is a human growth hormone polyadenylation sequence (hGHpA) includes SEQ ID NO: 18. Other technical features may be readily apparent to one skilled in the art from the following figures, descriptions, and claims.

In another aspect, provided herein is an infectious recombinant adeno-associated virus (rAAV) particle comprising (i) a capsid protein and (ii) a vector genome comprising a polynucleotide sequence encoding a channelrhodopsin, wherein: a woodchuck hepatitis virus posttranscriptional regulatory element (WPRE) is positioned downstream of the polynucleotide sequence encoding the channelrhodopsin; the vector genome does not encode a fluorescent protein; or the capsid protein is an AAV2 7m8 serotype. In some embodiments, the woodchuck hepatitis virus posttranscriptional regulatory element (WPRE) is positioned downstream of the polynucleotide sequence encoding the channelrhodopsin. In some embodiments, the WPRE element comprises SEQ ID NO: 14 or a sequence having at least 90%, at least 95%, at least 98%, or at least 99% sequence identity thereto.

In some embodiments, the vector genome does not encode a fluorescent protein.

In some embodiments, the capsid protein is an AAV2 7m8 serotype. In some embodiments, the capsid protein comprises the amino acid sequence of SEQ ID NO: 7.

In some embodiments, the polynucleotide encoding the channelrhodopsin encodes the channel rhodopsin of SEQ ID NO: 5. In some embodiments, the polynucleotide encoding the channelrhodopsin encodes an amino acid sequence having at least 80%, at least 85%, at least 90%, at least 95%, at least 97%, at least 98%, or at least 99% sequence identity to SEQ ID NO: 5. In some embodiments, the polynucleotide encoding the channelrhodopsin comprises SEQ ID NO: 17.

In some embodiments, the vector further comprises an upstream inverted terminal repeat (ITR), wherein the upstream ITR comprises SEQ ID NO: 15. In some embodiments, the vector further comprises a downstream ITR, wherein the downstream ITR comprises SEQ ID NO: 16.

In some embodiments, the vector further comprises a polyadenylation sequence. In some embodiments, the polyadenylation sequence is a human growth hormone polyadenylation sequence (hGHpA) or a Simian virus 40 polyadenylation sequence. In some embodiments, the polyadenylation sequence comprises SEQ ID NO: 18.

In some embodiments, the vector further comprises a promoter. In some embodiments, the promoter comprises a cytomegalovirus (CMV) promoter, an elongation factor 1a (EF1a) promoter, a simian virus 40 (SV40) promoter, a chicken beta-actin promoter, an mGluR6 promoter, or a CAG promoter. In some embodiments, the promoter comprises SEQ ID NO: 10. In some embodiments, the vector comprises SEQ ID NO: 34. In some embodiments, the vector comprises SEQ ID NO: 29. In some embodiments, the vector comprises an mGluR6 regulatory element region, wherein the mGluR6 regulatory element region comprises the promoter. In some embodiments, the mGluR6 regulatory element comprises at least one of, at least two of, at least three of, or all four of SEQ ID NOS: 24-27. In some embodiments, the mGluR6 regulatory element comprises, from upstream to downstream: intron 4 of the mGluR6 gene, intron 3 of the mGluR6 gene, an mGluR6 enhancer, and a fragment of the mGluR6 promoter. In some embodiments, the mGluR6 regulatory element comprises SEQ ID NO: 28.

In some embodiments, the vector comprises SEQ ID NO: 30. In some embodiments, the vector comprises SEQ ID NO: 31.

In some embodiments, the vector comprises, from upstream to downstream, (a) an AAV2 inverted terminal repeat sequence (ITR1); (b) a promoter sequence; (c) a polynucleotide sequence encoding the channelrhodopsin; (d) a polyadenylation sequence; and (e) an AAV2 inverted terminal repeat sequence (ITR2). In some embodiments, the vector comprises a woodchuck hepatitis virus posttranscriptional regulatory element (WPRE) positioned between the polyadenylation sequence and the ITR2 sequence.

A pharmaceutical composition includes a plurality of the rAAV particles and may also include and a pharmaceutically acceptable carrier or excipient. The pharmaceutical composition may also include where the composition is formulated for intravitreal injection. The pharmaceutical composition may also include where the composition is formulated as an emulsion or suspension. Other technical features may be readily apparent to one skilled in the art from the following figures, descriptions, and claims.

Also provided are methods for delivering a heterologous nucleic acid to a retinal cell where the methods include contacting the retinal cell with a plurality of the rAAV particles as described herein.

Also provided are methods for treating a retinal disease in a human subject in need thereof, the methods comprising administering to the subject a therapeutically effective amount a pharmaceutical composition comprising a plurality of the rAAV particles described herein. The pharmaceutical composition may also include a pharmaceutically acceptable carrier or excipient. The method may also include where the rAAV particles are administered by intravitreal injection.

The method may also include where the rAAV particles are administered in one or more doses. In aspects, the one or more doses comprises at least about 1.5×10⁸ viral genomes (vg). In aspects, the one or more doses comprises about 1×10⁸ to 1×10¹⁴ viral genomes (vg).

The method may also include where the rAAV particles are administered in one or more doses per eye.

The method may also include where the retinal disease is selected from Bardet-Biedl syndrome, chorioretinal atrophy or degeneration, cone or cone-rod dystrophy, congenital stationary night blindness, Leber congenital amaurosis (LCA), macular degeneration (MD), including age-related MD (AMD), ocular-retinal developmental disease, optic atrophy, retinitis pigmentosa, syndromic/systemic diseases with retinopathy, Usher syndrome, or other retinopathy, including diabetic retinopathy. The method may also include where the retinal disease is age-related macular degeneration (AMD) or retinitis pigmentosa (RP). Other technical features may be readily apparent to one skilled in the art from the following figures, descriptions, and claims.

### BRIEF DESCRIPTION OF THE FIGURES

FIG. 1A shows ChRown mediated spiking activities in response to the incremental light intensity, light intensities measured as photons/cm²s are shown in the upper right of each trace.

FIG. 1B shows ChRown-GFP mediated spiking activities in response to the incremental light intensity, light intensities measured as photons/cm²s are shown in the upper right of each trace.

FIG. 1C shows light-evoked spikes recorded from a single electrode of a ChRown treated retina.

FIG. 1D shows light-evoked spikes recorded from a single electrode of a ChRown-GFP treated retina.

FIG. 1E shows raster plots of 30 consecutive light-elicited spikes originated from a single neuron (30 trials and 10 min recordings) of a ChRown treated retina.

FIG. 1F shows raster plots of 30 consecutive light-elicited spikes originated from a single neuron (30 trials and 10 min recordings) of a ChRown-GFP treated retina.

FIG. 1G shows the averaged spike rate histograms from the light-elicited spikes in FIG. 1E.

FIG. 1H shows the averaged spike rate histograms from the light-elicited spikes in FIG. 1F.

FIG. 2 shows results from a visual acuity test with two doses (low, high) of either pCAG-ChRown-GFP-mWPRE-hGHpA (R01) or pCAG-ChRown-mWPRE-hGHpA (R03). Average values +/−SD for n=6 animals/group.

FIG. 3A is a line graph showing total clinical score of slit lamp examinations. Data are presented as mean±SEM. Test articles were vehicle (large circle); AAV2 (wt) (square); AAV2 (7m8) (triangle); AAV2 (7m8) high dose (diamond); AAV8 (small circle); and AAV8 high dose (star). See Table 3 for doses.

FIG. 3B is a line graph showing keratic precipitates based on slit lamp examinations. Data are presented as mean±SEM. Test articles were vehicle (small circle); AAV2 (wt) (square); AAV2 (7m8) (triangle); AAV2 (7m8) high dose (inverted triangle); AAV8 (large circle); and AAV8 high dose (diamond). See Table 3 for doses.

FIG. 3C is a line graph showing iris hyperemia based on slit lamp examinations. Data are presented as mean±SEM. Test articles were vehicle (small circle); AAV2 (wt) (square); AAV2 (7m8) (triangle); AAV2 (7m8) high dose (inverted triangle); AAV8 (large circle); and AAV8 high dose (diamond). See Table 3 for doses.

FIG. 3D is a line graph showing anterior lens capsule deposit based on slit lamp examinations. Posterior lens capsule deposits were similar (data not shown). Data are presented as mean±SEM. Test articles were vehicle (small circle); AAV2 (wt) (square); AAV2 (7m8) (triangle); AAV2 (7m8) high dose (inverted triangle); AAV8 (large circle); and AAV8 high dose (diamond). See Table 3 for doses.

FIG. 4A is a line graph showing GFP expression in the foveal position of the eye globe, graded from fluorescent fundus images. Data are presented as mean±SEM. Test articles were AAV2 at 1×10¹¹ (square); AAV2 7m8 low dose (triangle); AAV2 7m8 high dose (inverted triangle); and AAV8 high dose (circle).

FIG. 4B is a line graph showing GFP expression in the peripheral position of the eye globe, graded from fluorescent fundus images. Data are presented as mean±SEM. Test articles were AAV2 at 1×10¹¹ (square); AAV2 7m8 low dose (triangle); AAV2 7m8 high dose (inverted triangle); and AAV8 high dose (circle).

FIG. 4C is a line graph showing GFP expression in the perivascular position of the eye globe, graded from fluorescent fundus images. Data are presented as mean±SEM and test articles were as in FIG. 4A.

FIG. 5 shows representative infrared and autofluorescence cSLO images obtained at Day 42 for eyes treated with Vehicle, AAV2, AAV 7m8 low dose, AAV2 7m8 high dose, AAV8 high dose, and AAV8 low dose. GFP expression was limited across most treatment groups, except AAV2 7m8 low dose and AAV2 7m8 high dose where expression ranged from moderate to bright.

FIG. 6 is a line graph showing transgene expression (copies target per microgram RNA) in selected optical tissues of non-human primates at three doses. See Example 6 for detail.

FIG. 7 shows the relationship between light sensitivity and viral vector dose. Light sensitivity was defined as the lowest light intensity that evoked OMR at a grating frequency of 0.042 cycle/degree. Data are presented as means±SD (n=3-4 mice in each group). Light intensity values among the groups with viral dose≥7.5×10⁸ vg/eye are not significantly different (p>0.05; one-way ANOVA). The dashed line indicates the estimated maximum light intensity of the OptoDrum, which was used to measure visual acuity.

FIG. 8 shows the relationship between visual acuity and viral vector dose. Visual acuity was determined as the highest grating frequency at 100% contrast that evoked OMR. Data are presented as means±SD (n=3-4 mice in each group). Visual acuity values among the groups with viral dose≥1.5×10⁸ vg/eye are not significantly different (p>0.05; one-way ANOVA).

FIG. 9 shows representative immunofluorescence images of ChRown (fused to GFP) in retinal wholemounts from treated TKO mice. Viral transduced RGCs were determined by GFP labeling. RGCs were labeled with an antibody against RBPMS, a RGC-specific marker.

FIG. 10A shows the relationship between total RGCs and viral vector dose. RGC densities among all the treated groups were not statistically different (p>0.05; one-way ANOVA).

FIG. 10B shows the relationship between viral vector dose and the normalized density of transduced RGCs. The data are presented as means±SD (n=4 retinas in each dose group).

FIG. 11A shows representative Western blot images of ChRown-GFP protein bands from whole retinas, labeled using an antibody against GFP. β-actin served as control.

FIG. 11B shows the relative ECL fluorescence intensity of ChRown-GFP after normalization by the ECL fluorescence intensity of β-actin. Four retinas were used for each viral dose group. Data are presented as means±SD (n=3 experiments).

FIG. 12A shows the relationship between light sensitivity and viral vector dose two months after injection. Light sensitivity was defined as the lowest light intensity that evoked OMR at a grating frequency of 0.042 cycle/degree. Data are presented as means±SD (n=6 mice in each group).

FIG. 12B shows the relationship between light sensitivity and viral vector dose six months after injection. Light sensitivity was defined as the lowest light intensity that evoked OMR at a grating frequency of 0.042 cycle/degree. Data are presented as means±SD (n=6 mice in each group).

FIG. 13A shows the relationship between visual acuity and viral vector dose two months after injection. Visual acuity was determined as the highest grating frequency at 100% contrast that evoked OMR. Data are presented as means±SD (n=6 mice in each group).

FIG. 13B shows the relationship between visual acuity and viral vector dose six months after injection. Visual acuity was determined as the highest grating frequency at 100% contrast that evoked OMR. Data are presented as means±SD (n=6 mice in each group).

FIG. 14A shows the relationship between light sensitivity and spatial frequency for the TKO mice treated with ChRown (n=6). Light sensitivity is defined as the threshold light intensity that was required to evoke optomotor response.

FIG. 14B shows that light sensitivity as assessed at the spatial frequency of 0.042 cycle/degree (which is near the peak sensitive frequency) was stable up to 10 months after viral vector injection (n=5).

FIG. 14C shows that visual acuity was stable for up to 10 months after viral vector injection (n=5).

FIG. 14D shows the relationship between contrast sensitivity and spatial frequency (n=5).

### DETAILED DESCRIPTION

Photoreceptor loss or degeneration underlies a number of human diseases characterized by vision loss including age-related macular degeneration (AMD) and retinitis pigmentosa (RP). Loss of photoreceptor cells and/or loss of a photoreceptor cell function are the primary causes of diminished visual acuity, diminished light sensitivity, and blindness in humans. The present invention addresses the need for additional therapeutic agents for the treatment of retinal diseases where photoreceptor loss or degeneration is a factor by providing a therapeutic transgene encoding a light-sensitive protein delivered to retinal cells using a recombinant adeno-associated virus (rAAV) vehicle as described herein, and related compositions and methods for the treatment of retinal diseases and disorders.

The rAAV vehicle advantageously delivers the transgene to retinal cells, which may include retinal photoreceptor cells, retinal ganglion cells, and bipolar cells as well as retinal amacrine cells, horizontal cells, Müller cells, and retinal pigment epithelial cells.

In embodiments, the rAAV vectors provide targeted delivery to photoreceptor cells, or to a combination of photoreceptor cells, retinal ganglion cells, and bipolar cells. Photoreceptor cells are highly specialized neurons responsible for the conversion of light into electrical and chemical signals that collectively with the activity of ganglion and bipolar cells propagate these signals to the brain which generates a visual representation. Bipolar cells receive input from photoreceptor cells and pass the electrical signals on to ganglion cells, whose axons collectively form the optic nerve.

Photoreceptor cells include rod and cone cells which contain rhodopsin and cone opsins, a light-sensitive protein. Rhodopsin, like other opsins, is a G-protein-coupled receptor (GPCR) embedded in the lipid bilayer of the cell membranes and having seven transmembrane domains forming a binding pocket for its ligand, 11-cis-retinal. Signaling is initiated when retinal absorbs a photon of light, resulting in its isomerization to all-trans-retinal and activating a series of reactions referred to as the phototransduction cascade. This signaling results in the movement of ions across the cell membrane, resulting in it becoming electrically polarized, creating a series of electrical and chemical signals that are ultimately conveyed to the brain.

Visual information is processed through the retina through two pathways: an ON pathway which signals the light ON, and an OFF pathway which signals the light OFF. The existence of the ON/OFF pathways is important for enhancement of contrast sensitivity. The visual signal in the ON pathway is relayed from ON cone bipolar cells to ON ganglion cells. Both ON cone bipolar cells and ON ganglion cells are depolarized in response to light. On the other hand, the visual signal in the OFF pathway is carried from OFF cone bipolar cells to OFF ganglion cells. Both OFF cone bipolar cells and OFF ganglion cells are hypopolarized in response to light. Rod bipolar cells, which are responsible for the ability to see in dim light (scotopic vision), are ON bipolar cells (depolarized in response to light). Rod bipolar cells relay the vision signal through All amacrine cells (an ON type of retinal cell) to ON and OFF cone bipolar cells.

In embodiments, the invention provides methods of treating a retinal disease comprising expression of a channelrhodopsin transgene in retinal cells of a subject having photoreceptor loss or degeneration utilizing the compositions and methods described here. Channelrhodopsins are a subfamily of retinylidene proteins (rhodopsins) originally identified in algae where they serve as sensory photoreceptors. Heterologous expression in mammalian cells is associated with light-sensitive electrical signaling, calcium influx, etc.

### Transgene

In accordance with the various embodiments of the compositions and methods described here, the transgene is a channelrhodopsin. In embodiments, the channelrhodopsin is a variant of a wild-type Chloromonas oogama channelrhodopsin, wherein the variant has improved light sensitivity compared to a reference protein, which may be a native or wild-type protein. The terms “native” and “wild-type” with reference to a protein are used interchangeably and refer to the naturally occurring protein.

The wild-type Chloromonas oogama channelrhodopsin is described in Klapoetke et al., 2014 Nat. Methods 11(3): 338-46. Chloromonas oogama channelrhodopsin variants are described in U.S. Pat. No. 10,392,426 (Klapoetke et al.) and U.S. Pat. No. 11,041,004 (Pan et al.).

The amino acid and nucleotide sequences of the wild-type Chloromonas oogama channelrhodopsin are represented by SEQ ID NO: 1 and SEQ ID NO: 2, respectively.

Improved light sensitivity can be demonstrated, for example, by comparing the ion flux and/or proton flux across a membrane produced by recombinantly expressed proteins subjected to activating light. In this context, “activating light” refers to light energy which is above the threshold for activation of the protein. In the context of the variant channelrhodopsins described here, the activating light is approximately 470 nm. In some embodiments, the activating light has a wavelength between about 450 nm to about 495 nm, which may also be referred to as blue light.

The variant channelrhodopsins utilized as transgenes here were identified by screening a library of rationally designed site-specific mutants of Chloromonas oogama. The variants described here are optimized for visual function including high light sensitivity and advantageous channel kinetics and are described in U.S. Pat. No. 11,041,004 (Pan et al.) and Ganjawala, T. H., et al., Improved CoChR Variants Restore Visual Acuity and Contrast Sensitivity in a Mouse Model of Blindness under Ambient Light Conditions. Mol Ther, 2019. 27(6): p. 1195-1205.

Unlike the channelrhodopsin transgenes described previously, the transgenes utilized in the compositions and methods described here advantageously are expressed as the channelrhodopsin protein alone, rather than a fusion protein of the channelrhodopsin with a fluorescent reporter protein, such as a green fluorescent protein (“GFP”) or similar. All previous preclinical and clinical studies on optogenetic restoration of sensory function have been performed using channelrhodopsins fused at their C-terminus to a fluorescent protein. Further, it has been reported that removing the fluorescent protein tag results in a “massive” reduction of photocurrent in transfected cells (see, e.g., Zerche et al., Mol Ther Methods Clin Dev 2023 Mar. 21: 29:202-212 and Gauvain et al., Commun Biol. 2021; 4:125). Further, while some reports indicated that the fusion protein provided higher transgene expression in certain constructs, possibly by stabilizing the expressed protein against degradation, the present inventors unexpectedly found no difference in expression, localization or function between constructs comprising the therapeutic transgene alone and those comprising a fusion protein of the therapeutic transgene and GFP, as discussed in the examples below.

In embodiments, the transgene comprises a variant channelrhodopsin represented by SEQ ID NO: 3.

In embodiments, the transgene comprises a variant channelrhodopsin represented by SEQ ID NO: 4.

In embodiments, the transgene is a variant of SEQ ID NO: 3 or SEQ ID NO: 4 having an amino acid sequence that is at least 80%, at least 85%, at least 90%, at least 95%, at least 97%, at least 98%, or at least 99% identical to SEQ ID NO: 3 or SEQ ID NO: 4.

In embodiments, the amino acid sequence of the variant channelrhodopsin is represented by SEQ ID NO: 5, referred to herein as ChRown. Unlike other channelrhodopsin-based gene therapies being evaluated for clinical use, ChRown is not a fusion protein comprising the therapeutic transgene fused with a fluorescent reporter protein (FP) such as the Green Fluorescent Protein (GFP) or similar.

In embodiments, the transgene is a variant of ChRown having an amino acid sequence that is at least 80%, at least 85%, at least 90%, at least 95%, at least 97%, at least 98%, or at least 99% identical to SEQ ID NO: 5.

Generally, the transgene sequence can be from about 2 to 5 kb in length, although this size may be made up of the transgene and additional non-coding sequences or additional copies of the transgene, for example separated by a ribosome readthrough or an internal ribosome entry site, or “IRES”.

### Gene Delivery Vehicle

Adeno-associated virus (AAV) is a non-enveloped virus of the Parvoviridae family that requires a helper virus to propagate and is therefore considered non-pathogenic. There are currently 12 AAV serotypes that have been identified, each defined by unique capsid proteins. The different serotypes also exhibit differences in cellular tropism, transduction efficiency, and immunogenicity. Serotypes AAV1, AAV2, AAV4, AAV5, AAV6, AAV7, AAV8 and AAV9 have been reported to shown tropism for retinal cells.

The present invention provides recombinant adeno-associated virus (rAAV) vehicles for delivery and expression of a therapeutic transgene to target retinal cells. The term “AAV vehicle” refers to an infectious recombinant viral particle comprising (i) a capsid protein encapsulating (ii) a vector genome consisting of a heterologous polynucleotide encoding the transgene. The heterologous polynucleotide encoding the transgene may also be referred to as an rAAV vector. The rAAV vector also contains additional heterologous polynucleotide sequences upstream and downstream of the transgene. For example, as described in more detail below, the rAAV vector may include non-AAV promoter sequences, enhancer sequences, and termination/polyadenylation sequences. The vector is constructed such that these heterologous sequences are flanked by two AAV inverted terminal repeat sequences (ITRs). The ITR generally consists of nucleotides 1 to 145 at the 5′end of the AAV DNA genome, and nucleotides 4681 to 4536 at the 3′end of the AAV DNA genome. The rAAV vector may also include at least 10 nucleotides following the end of the ITR (e.g., a portion of the “D region”).

In embodiments, the rAAV vector comprises a 5′ ITR having a nucleotide sequence of SEQ ID NO: 15 and a 3′ ITR having a nucleotide sequence of SEQ ID NO: 16.

The capsid is formed from structural proteins which may include 1-3 of the structural proteins encoded by the AAV Cap open reading frame, referred to as VP1, VP2, and VP3. The viral particles are formed during AAV production using helper plasmids containing the AAV Rep and Cap open reading frames. The Rep reading frame encodes proteins that regulate replication. In embodiments, the serotype of the rAAV vehicle is AAV2 and the recombinant viral particles comprise a modified VP1 capsid protein.

The rAAV vehicles described here advantageously infect primate retinal cells and transfer the vector genome comprising the therapeutic channelrhodopsin transgene into the retinal cells where the transgene is expressed at high levels and in the appropriate subcellular structure, i.e., the plasma membrane of the cell, such that the expressed channelrhodopsin protein functions to generate a flux of ions through the channel in response to activating light. The resulting ion flux results in depolarization of the neuronal cell which in turn results in an electrical signal being transmitted, thereby creating an electrical signal in response to activating light.

The term “retinal cells” may include any of the cell types that comprise the retina, including retinal photoreceptor cells, retinal ganglion cells, and bipolar cells as well as retinal amacrine cells, horizontal cells, Müller cells, and retinal pigment epithelial cells. In embodiments, the rAAV vectors provide targeted delivery to photoreceptor cells, or to a combination of photoreceptor cells, retinal ganglion cells, and bipolar cells.

In some embodiments, self-complementary AAV vectors may be used. These vectors feature an inverted repeat genome that can fold into double-stranded DNA (dsDNA) without the requirement for DNA synthesis or base-pairing between multiple vector genomes.

In embodiments, the rAAV vehicle comprises a capsid protein having an amino acid sequence that is modified relative to a native AAV capsid sequence (e.g., SEQ ID NO: 37). In this context the term “modified” refers to an insertion, substitution, or deletion of one or more amino acids relative the sequence of an AAV capsid of serotype 2.

In embodiments, the rAAV vehicle comprises a modified AAV2 VP1 capsid protein having the amino acid sequence of SEQ ID NO: 6, wherein the modified AAV2 VP1 contains a peptide insertion at amino acid position 588 of the wild-type AAV2 capsid sequence and, excluding the peptide insertion, has an amino acid sequence at least 90%, at least 95%, at least 98%, or at least 99% identical to SEQ ID NO: 6.

In embodiments, the rAAV vehicle comprises a modified AAV2 VP1 capsid protein having an amino acid sequence containing an insertion of a peptide represented by SEQ ID NO: 8 (L G E T T R P) at an amino acid corresponding to the amino acid at position 588 of the wild-type AAV2 VP1 capsid sequence.

In embodiments, the rAAV vehicle comprises a modified AAV2 VP1 capsid protein having the amino acid sequence of SEQ ID NO: 38, wherein the modified AAV2 VP1 contains a peptide insertion at amino acid position 588 of the wild-type AAV2 capsid sequence and, excluding the peptide insertion, has an amino acid sequence at least 90%, at least 95%, at least 98%, or at least 99% identical to SEQ ID NO: 38.

In embodiments, the rAAV vehicle comprises a modified AAV2 VP1 capsid protein having an amino acid sequence containing an insertion of a peptide represented by SEQ ID NO: 9 (L A I S D Q T K H A) at an amino acid corresponding to the amino acid at position 588 of the wild-type AAV2 VP1 capsid sequence.

In some embodiments, the rAAV vehicle comprises a modified AAV2 VP1 capsid protein having an amino acid sequence containing an amino acid substitution at position Y444 corresponding to position the wild-type AAV2 VP1 capsid sequence. In some embodiments, the amino acid substitution is Y444F.

In some embodiments, the rAAV vehicle comprises a modified AAV2 VP1 capsid protein having an amino acid sequence containing an insertion of a peptide represented by SEQ ID NO: 8 (L GET TR P) at an amino acid corresponding to the amino acid at position 588 of the wild-type AAV2 VP1 capsid sequence and further comprises an amino acid substitution at position Y444 corresponding to position the wild-type AAV2 VP1 capsid sequence. In some embodiments, the amino acid substitution is Y444F.

In some embodiments, the rAAV vehicle comprises a modified AAV2 VP1 capsid protein having an amino acid sequence containing an insertion of a peptide represented by SEQ ID NO: 9 (L A I S D Q T K H A) at an amino acid corresponding to the amino acid at position 588 of the wild-type AAV2 VP1 capsid sequence and further comprises an amino acid substitution at position Y444 corresponding to position the wild-type AAV2 VP1 capsid sequence. In some embodiments, the amino acid substitution is Y444F.

In embodiments, the rAAV vehicle comprises a capsid protein having an amino acid sequence that is modified relative to a native AAV capsid protein of serotype 2 and a polynucleotide comprising from 5′ to 3′ an AAV2 terminal repeat sequence; a promoter sequence; a polynucleotide sequence encoding an opsin; a polyadenylation sequence; and an AAV2 terminal repeat sequence.

In some embodiments, the promoter is selected from a cytomegalovirus (CMV) promoter, an elongation factor 1a (EF1a) promoter, a simian virus 40 (SV40) promoter, a chicken beta-actin promoter, an mGluR6 promoter, and a CAG promoter.

A CAG promoter comprises three elements, (C) the cytomegalovirus (CMV) early enhancer element, (A) the promoter, first exon and first intron of the chicken beta-actin gene, and (G) the splice acceptor of the rabbit beta-globin gene. In embodiments, the CAG promoter is a modified CAG promoter of SEQ ID NO: 10, or a sequence having at least 95%, at least 98%, or at least 99% identity thereto.

In some embodiments, the promoter comprises a modified metabotropic glutamate receptor (mGluR6) promoter. In embodiments, the promoter comprises a fragment of the human mGluR6 promoter region identified by SEQ ID NO: 11 or SEQ ID NO: 12, or a nucleotide sequence having at least 80%, at least 90%, at least 95%, at least 98%, or at least 99% identity thereto. In some embodiments, the promoter comprises a fragment of the human mGluR6 promoter region identified by SEQ ID NO: 24, or a nucleotide sequence having at least 80%, at least 90%, at least 95% at least 98% or at least 99% identity thereto.

In some embodiments, the vector comprises an mGluR6 regulatory element region, wherein the mGluR6 regulatory element region comprises an mGluR6 promotor or fragment thereof. In some embodiments, the mGluR6 regulatory element region further comprises an enhancer sequence identified by SEQ ID NO: 13, or a nucleotide sequence having at least 80%, at least 90%, at least 95%, at least 98%, or at least 99% identity thereto. In some embodiments, the mGluR6 regulatory element region further comprises an enhancer sequence identified by SEQ ID NO: 25, or a nucleotide sequence having at least 80%, at least 90%, at least 95%, at least 98%, or at least 99% identity thereto. In some embodiments, the mGluR6 regulatory element region may further comprise one or more mGluR6 intron sequences identified by one or both of SEQ ID NO: 26 or SEQ ID NO: 27, or a nucleotide sequence having at least 80%, at least 90%, at least 95%, at least 98%, or at least 99% identity thereto.

In some embodiments, the mGluR6 regulatory element region comprises a fragment of the human mGluR6 promoter, an mGluR6 enhancer, intron 4 of the mGluR6 gene, and/or intron 3 of the mGluR6 gene. In some embodiments, the promoter comprises, from upstream to downstream, intron 4 of the mGluR6 gene, intron 3 of the mGluR6 gene, an mGluR6 enhancer, and a fragment of the mGluR6 promoter. In some embodiments, the promoter comprises, from 5′ to 3′, SEQ ID NO: 26, SEQ ID NO: 27, SEQ ID NO: 25, and SEQ ID NO: 24. In some embodiments, the promoter comprises SEQ ID NO: 28, or a sequence having at least 95%, at least 98%, or at least 99% identity thereto.

In some embodiments, the rAAV vehicle comprises a WPRE enhancer element in combination with a CAG promoter.

In some embodiments, the rAAV vehicle comprises (i) a modified AAV2 VP1 capsid protein having the amino acid sequence of SEQ ID NO: 6, wherein the modified AAV2 VP1 contains a peptide insertion at an amino acid position corresponding to position 588 of the wild-type AAV2 capsid sequence and, excluding the peptide insertion, has an amino acid sequence at least 90%, at least 95%, at least 98%, or at least 99% identical to SEQ ID NO: 6, wherein the peptide insertion comprises SEQ ID NO: 8 or SEQ ID NO: 9; and (ii) a rAAV vector encoding ChRown SEQ ID NO: 5 operably linked to a modified CAG promoter according to SEQ ID NO: 10, and a WPRE enhancer according to SEQ ID NO: 14. In some embodiments, the ChRown according to SEQ ID NO: 5 may further be operably linked to a polyadenylation sequence according to SEQ ID NO: 18. The polyadenylation sequence according to SEQ ID NO: 18 may be positioned downstream of the WPRE enhancer according to SEQ ID NO: 14.

In some embodiments, the rAAV vehicle comprises a VP1 capsid protein comprising SEQ ID NO: 7 and a rAAV vector encoding ChRown (SEQ ID NO: 5) operably linked to a modified CAG promoter (SEQ ID NO: 10) and a modified WPRE enhancer (SEQ ID NO: 14). In some embodiments, an rAAV vector encoding ChRown operably linked to a modified CAG promoter, a modified WPRE enhancer and a human growth hormone polyadenylation sequence (hGHpA) is represented by SEQ ID NO: 34 (pCAG-Chrown-mWPRE-hGHpA). In some embodiments, the rAAV vector further comprises a 5′ ITR. In some embodiments, the 5′ ITR is represented by SEQ ID NO: 15. In some embodiments, the rAAV vector further comprises a 3′ ITR. In some embodiments, the 3′ ITR is represented by SEQ ID NO: 16. In some embodiments, the rAAV vector comprises or consists of SEQ ID NO: 29.

In some embodiments, the rAAV vehicle comprises a VP1 capsid protein having the amino acid sequence of SEQ ID NO: 38 and a rAAV vector encoding ChRown (SEQ ID NO: 5) operably linked to a modified CAG promoter (SEQ ID NO: 10) and a modified WPRE enhancer (SEQ ID NO: 14). In some embodiments, an rAAV vector encoding ChRown operably linked to a modified CAG promoter, a modified WPRE enhancer and a human growth hormone polyadenylation sequence (hGHpA) is represented by SEQ ID NO: 34 (pCAG-Chrown-mWPRE-hGHpA). In some embodiments, the rAAV vector further comprises a 5′ITR. In some embodiments, the 5′ITR is represented by SEQ ID NO: 15. In some embodiments, the rAAV vector further comprises a 3′ITR. In some embodiments, the 3′ITR is represented by SEQ ID NO: 16. In some embodiments, the rAAV vector comprises or consists of SEQ ID NO: 29.

In some embodiments, the rAAV vehicle comprises (i) a modified AAV2 VP1 capsid protein having the amino acid sequence of SEQ ID NO: 6, wherein the modified AAV2 VP1 contains a peptide insertion at an amino acid position corresponding to position 588 of the wild-type AAV2 capsid sequence and, excluding the peptide insertion, has an amino acid sequence having at least 90%, at least 95%, at least 98%, or at least 99% sequence identity to SEQ ID NO: 6, wherein the peptide insertion comprises SEQ ID NO: 8 or SEQ ID NO: 9; and (ii) a rAAV vector encoding ChRown SEQ ID NO: 5 operably linked to an mGluR6 promoter according to SEQ ID NO: 28, a WPRE enhancer according to SEQ ID NO: 14, and a polyadenylation sequence according to SEQ ID NO: 18.

In some embodiments, the rAAV vehicle comprises a VP1 capsid protein comprising SEQ ID NO: 7 and a rAAV vector encoding ChRown SEQ ID NO: 5 operably linked to an mGluR6 promoter SEQ ID NO: 28 and a modified WPRE enhancer SEQ ID NO: 14. In embodiments, an rAAV vector encoding ChRown operably linked to an mGluR6 promoter, a modified WPRE enhancer and a human growth hormone polyadenylation sequence (hGHpA) is represented by SEQ ID NO: 30 (mGluR6-Chrown-mWPRE-hGHpA). In some embodiments, the rAAV vector further comprises a 5′ ITR. In some embodiments, the 5′ ITR is represented by SEQ ID NO: 15. In some embodiments, the rAAV vector further comprises a 3′ ITR. In some embodiments, the 3′ ITR is represented by SEQ ID NO: 16. In some embodiments, the rAAV vector comprises or consists of SEQ ID NO: 31.

In some embodiments, the rAAV vehicle comprises a VP1 capsid protein having the amino acid sequence of SEQ ID NO: 38 and a rAAV vector encoding ChRown SEQ ID NO: 5 operably linked to an mGluR6 promoter SEQ ID NO: 28 and a modified WPRE enhancer SEQ ID NO: 14. In embodiments, an rAAV vector encoding ChRown operably linked to an mGluR6 promoter, a modified WPRE enhancer and a human growth hormone polyadenylation sequence (hGHpA) is represented by SEQ ID NO: 30 (mGluR6-Chrown-mWPRE-hGHpA). In some embodiments, the rAAV vector further comprises a 5′ ITR. In some embodiments, the 5′ ITR is represented by SEQ ID NO: 15. In some embodiments, the rAAV vector further comprises a 3′ ITR. In some embodiments, the 3′ ITR is represented by SEQ ID NO: 16. In some embodiments, the rAAV vector comprises or consists of SEQ ID NO: 31.

In some embodiments, the rAAV vector encoding ChRown comprises any one of CAG-ChRown-GFP-mWPRE-hGHpA (SEQ ID NO: 32), CAG-ChRown-GFP-hGHpA (SEQ ID NO; 33), CAG-ChRown-Mwpre-hGHpA (SEQ ID NO: 34), ChRown-tdTomato-hGHpA (SEQ ID NO: 35), and CAG-ChRown-hGHpA (SEQ ID NO: 36). In some embodiments, the rAAV vector encoding ChRown further comprises a 5′ ITR and a 3′ ITR. In some embodiments, the 5′ ITR is represented by SEQ ID NO: 15 and the 3′ ITR is represented by SEQ ID NO: 16.

Other vectors encoding ChRown include pCAG-Chrown-GFP-mWPRE-hGHpA (SEQ ID NO: 19), pCAG-Chrown-GFP-hGHpA (SEQ ID NO: 20), pChrown-tdTomato-hGHpA (SEQ ID NO: 22), and pCAG-Chrown-hGHpA (SEQ ID NO: 23).

In some embodiments, the present invention provides a gene delivery vehicle in the form of an infectious recombinant adeno-associated virus (rAAV) particle comprising (i) a capsid protein having a modified amino acid sequence relative to a native AAV capsid of serotype 2 and (ii) a vector genome comprising or consisting of a heterologous polynucleotide comprising from 5′ to 3′ (a) an AAV2 inverted terminal repeat sequence (ITR1); (b) a promoter sequence; (c) a polynucleotide sequence encoding a channelrhodopsin; (d) a polyadenylation sequence; and (e) an AAV2 inverted terminal repeat sequence (ITR2), wherein the heterologous polynucleotide does not encode a fluorescent protein.

In some embodiments, the present invention provides a gene delivery vehicle in the form of an infectious recombinant adeno-associated virus (rAAV) particle comprising (i) a capsid protein having a modified amino acid sequence relative to a native AAV capsid of serotype 2 and (ii) a vector genome comprising or consisting of a heterologous polynucleotide comprising the nucleotide sequence of SEQ ID NO: 29, or a sequence having at least 90%, at least 95%, or at least 99% sequence identity thereto. In some embodiments, the capsid protein comprises or consists of SEQ ID NO: 38.

In some embodiments, the present invention provides a gene delivery vehicle in the form of an infectious recombinant adeno-associated virus (rAAV) particle comprising (i) a capsid protein having a modified amino acid sequence relative to a native AAV capsid of serotype 2 and (ii) a vector genome comprising or consisting of a heterologous polynucleotide comprising the nucleotide sequence of SEQ ID NO: 31, or a sequence having at least 90%, at least 95%, or at least 99% identity thereto. In some embodiments, the capsid protein comprises or consists of SEQ ID NO: 38.

### Methods of Manufacture

The rAAV vehicle described here may be produced, for example by transfecting a suitable cell line, such as HEK293 cells, with from two to three plasmids containing (1) the rAVV vector; (2) the AAV Rep and Cap genes; and optionally (3) helper genes, respectively. Other platforms may include the use of mammalian or insect cell lines stably expressing one or more of the viral helper genes, such as E1a and E1b or use of a baculovirus/insect cell system in which one to three baculovirus particles are used to infect the insect cells with e.g., the Rep gene, the Cap gene, and the rAVV vector containing the transgene. The insect cells may also be engineered to contain the viral Rep and Cap genes or the rAAV vector stably integrated into their genome.

The cells may be grown as adherent cultures or in suspension. For larger-scale culture volumes, virus can be produced in roller bottles, multilayer adherent culture flasks, continuous perfusion, or WAVE Bioreactor systems.

The methods may further include one or more purification and polishing steps downstream from cell culture and virus production. For example, affinity or heparin chromatography may be used to isolate virus from culture supernatants. The affinity resins may be based on AAV-specific binding proteins such as scFvs and antibody single domains from llamas (camelids). In addition, benzonase/DNAse treatment of eluted virus may be utilized for removal of extraviral DNA contamination. The methods may further include an anion-exchange chromatography step or density gradient centrifugation to separate empty AAV particles from those containing the rAAV vector.

In embodiments, the methods may include density gradient centrifugation through a density gradient selected from a continuous cesium chloride (CsCl) density gradient and an iodixanol step density gradient. The method may also include one or more of cell lysis and precipitation of cellular DNA and proteins prior to ultracentrifugation through the density gradient.

### Pharmaceutical Compositions

The disclosure provides pharmaceutical compositions comprising the rAAV vehicles described herein. In embodiments, the compositions comprise a pharmaceutically acceptable vehicle, diluent, carrier, and/or excipient. Suitable vehicles include aqueous vehicles such as water or buffered saline, e.g., phosphate buffered saline (“PBS”) or other suitable buffers to maintain physiologic pH, such acetate buffers, citrate buffers, phosphate buffers and borate buffers.

In embodiments, the pharmaceutical compositions may comprise one or more excipients suitable for ophthalmological use, including tonicity adjustors, viscosity regulators, co-solvents, and stabilizers.

Tonicity adjusters include sodium chloride, potassium chloride, dextran, cyclodextrins, mannitol, dextrose, glycerol, sorbitol, boric acid, borax and propylene glycol and combinations thereof.

Viscosity regulators include hydroxypropyl methylcellulose (HPMC), hydroxypropylcellulose (HPC), carboxymethylcellulose (CMC), methylcellulose (MC), hydroxyethylcellulose (HEC), cellulose and derivatives thereof, polycarbophil, polyoxyethylene glycol (PEG), hyaluronic acid (HA), amylase and derivatives thereof, amylopectins and derivatives thereof, dextran and derivatives thereof, polyvinylpyrrolidone (PVP), polyvinyl alcohol (PVA), and acrylic polymers such as derivatives of poly acrylic or polymethacrylic acid including hydroxylmethyl methacrylate (HEMA), carbomer and combinations thereof.

Suitable co-solvents include a polysorbate, polyoxyethylene glycol (PEG), or propylene glycol, and combinations thereof.

Suitable stabilizing agents include sucrose, sorbitol, glycerol, trehalose, or mannitol.

In embodiments, the pharmaceutical composition is formulated for administration by a suitable route of administration for transduction of retinal cells, including, for example, intravitreal, intraocular, or subretinal injection. Preferably, the route of administration is by intravitreal injection. All retinal neurons, including retinal ganglion cells, bipolar cells, horizontal cells, amacrine cells, and photoreceptor cells are known to be reasonably well-accessible to intravitreal injection as disclosed herein. Intravitreal and/or subretinal injection can provide the necessary access to the bipolar cells, especially in circumstances in which the photoreceptor cell layer is absent due to degeneration. Other routes which may be suitable include periocular routes such as retrobulbar, subtenons, or subconjunctival injections, intracameral injection, or suprachoroidal injection.

In embodiments, pharmaceutical compositions formulated for intravitreal injection may comprise one or more of a co-solvent, a tonicity agent, a buffering agent, and a stabilizing agent. The co-solvent may be, for example, polysorbate, polyoxyethylene glycol (PEG), or propylene glycol, and combinations thereof. The tonicity agent may be, for example, sodium or potassium chloride. The buffering agent may be a phosphate buffer, such as phosphate buffered saline, or PBS. The stabilizing agent may be sucrose, sorbitol, glycerol, trehalose, or mannitol.

In embodiments, the pharmaceutical composition is formulated to contain from 1×10¹¹ to 1×10¹⁵ particles per milliliter, or from 1×10⁸ to 1×10¹⁴ viral genomes per injection, e.g., in a volume of from 50-100 microliters.

### Methods of Treating

The present invention provides methods of treating a retinal disease by administering to a human subject in need of therapy for a retinal disease a pharmaceutical composition comprising an rAAV vehicle as described herein.

In embodiments, the retinal disease may be Bardet-Biedl syndrome, chorioretinal atrophy or degeneration, cone or cone-rod dystrophy, congenital stationary night blindness, Leber congenital amaurosis (LCA), macular degeneration (MD), including age-related MD (AMD) and inherited MD such as Stargardt disease, ocular-retinal developmental disease, optic atrophy, retinitis pigmentosa, syndromic/systemic diseases with retinopathy, Usher syndrome, or other retinopathy, including diabetic retinopathy.

In embodiments, the retinal disease is age-related macular degeneration (AMD). AMD is a common cause of vision loss resulting from degeneration of photoreceptor cells in the central part of the retina.

In embodiments, the retinal disease is Stargardt disease.

In embodiments, the retinal disease is retinitis pigmentosa (RP). RP is a genetic disorder resulting in degeneration of photoreceptor cells in the retina. Vision loss typically begins with damage to rod cells, which are responsible for detecting light and dark, and progresses to cone cells, which are responsible for color vision.

In embodiments, the retinal disease is Leber congenital amaurosis (LCA), which may also be referred to as Leber's hereditary optic neuropathy. LCA is a rare genetic disorder affecting men and resulting in rapid vision loss due to degeneration of photoreceptor cells in the retina.

In accordance with the methods described here, a therapeutically effective amount of an rAAV vehicle is administered to the subject in need of therapy. The therapeutically effective amount is an amount or dose sufficient to treat the disease or disorder, or sufficient to achieve a desired therapeutic outcome, for example, the amelioration or stabilization of the disease or disorder, or one or more clinical symptoms. In embodiments, the therapeutically effective amount is an amount or dose sufficient to improve one or more of visual acuity, visual field, navigation, motion detection, contrast sensitivity, light sensitivity, object detection, and light dark discrimination. In embodiments, the therapeutically effective amount is an amount or dose sufficient to improve activities of daily life and/or quality of life as ascertained by standardized questionnaire, patient reported outcomes (PROs).

The term “vision” includes the ability of the eye to detect light from the external environment and send a signal to the brain for perception. The compositions and methods described here may be used to improve or restore vision where the improvement or restoration in vision includes, for example, increases in light detection or perception, increased light sensitivity, an increase in the ability to discern the direction from which a light stimulus is coming, increased ability to detect differing brightness and contrast levels, increased ability to recognize the shape of a visual target, and increases in visual evoked potential or transmission from the retina to the cortex. Visual recovery may include placing the affected person on a low end of vision function by increasing an aspect of vision such as light sensitivity or visual evoked potential, without restoring full sight. Improvements in vision may include improvements in visual acuity, contrast sensitivity, the ability to perceive color, depth perception, visual field, and navigation as well as correlated electrophysiological responses as noted on the retinal electrogram and visual evoked potentials.

The degree of restoration of vision can be determined through the measurement of vision before and after administering a composition comprising a therapeutic transgene including but not limited that the methods described here. Vision can be measured, for example, as (1) a light detection response by the subject after exposure to a light stimulus; (2) a light projection response by the subject after exposure to a light stimulus; (3) light resolution by the subject of a light vs. dark patterned visual stimulus, which measures the subject's capability of resolving light vs dark patterned visual stimuli as evidenced, for example by the presence of demonstrable reliable optokinetically produced nystagmoid eye movements and/or related head or body movements that demonstrate tracking of the target and/or the presence of a reliable ability to discriminate a pattern visual stimulus and to indicate such discrimination by verbal or non-verbal means, including, for example pointing, or pressing a bar or a button; (4) visual acuity; (5) visual field testing, for example Humphrey visual field testing and Full Field Sensitivity Testing (FST); (6) navigation and light intensity measurement, for example multi-luminance mobility testing (MLMT); (7) contrast sensitivity, e.g. qCSF; (8) color vision testing or electrical recording from the retina and visual cortex e.g. electroretinogram and visual evoked potential, and/or dynamic anatomical imaging of the brain (functional magnetic resonance imaging (fMRI).

Thus, an improvement or restoration of vision may include increases in amplitude or kinetics of photocurrents or electrical responses in response to light stimulus in the retinal cells, increases in light sensitivity which may be indicated by a lower threshold light intensity required for initiating a photocurrent or electrical response in response to a light stimulus, increases in the number or amplitude of light-evoked spiking or spike firings, increases in light responses to the visual cortex, including visual evoked potential.

In embodiments, a therapeutically effective amount comprises from about 10¹⁰ to about 10¹⁵ rAAV infectious units or vector genomes (vg) in a volume of between about 25 and about 200 μl per injection, or about 50-100 microliters. The rAAV infectious units can be measured, for example, according to Del la Camara et al. Accurate Quantification of AAV Vector Genomes by Quantitative PCR, Genes (Basel). 2021 April; 12(4):601. Vector genome copies may be determined, for example, using a quantitative polymerase chain reaction (qPCR) based method. Reference standard materials (RSMs) for AAV serotype 2 are available from the American Type Culture Collection. Preferably, vector genomes are determined by a droplet digital PCR method. Other dosages and volumes, preferably within these ranges but possibly outside them, may be selected by the treating professional, taking into account the physical state of the subject (preferably a human), who is being treated, including, age, weight, general health, and the nature and severity of the particular ocular disorder.

In embodiments, a therapeutically effective amount comprises from 1×10¹¹ to 1×10¹⁵ virus particles per milliliter, or from 1×10⁸ to 1×10¹⁴ viral genomes per injection, e.g., in a volume of from 50-100 microliters. Accordingly, in embodiments, the pharmaceutical composition is formulated to contain from 1×10¹¹ to 1×10¹⁵ virus particles per milliliter, or from 1×10⁸ to 1×10¹⁴ viral genomes per injection, e.g., in a volume of from 50-100 microliters

In the context of the methods described here, the subject treated is one in need of therapy for a retinal disease. The subject is preferably human, although other mammalian subjects are contemplated including non-human primates. The term “patient” refers to a human subject manifesting with one or more clinical symptoms of a retinal disease. In some embodiments, the patient is a human subject who has been diagnosed with a retinal disease.

Treatment,” “treating,” and “treat” describe the management and care of a patient for the purpose of combating a retinal disease and includes the administration of a composition described herein to alleviate the symptoms or complications of a retinal disease, which may include vision restoration, which may be partial vision restoration or improved vision relative to vision before therapy was initiated. “Treating” refers to (1) taking steps to obtain beneficial or desired results, including clinical results such as an amelioration or reduction in one or more symptoms of the retinal disease; (2) inhibiting the retinal disease, for example, arresting or reducing the development or clinical progression of the disease or any one or more of its clinical symptoms; (3) relieving the disease, for example, causing regression of the disease or its clinical symptoms; or (4) delaying or slowing disease progression.

In the context of the present methods, the term “administering” when used in connection with a composition described herein may refer to direct administration or indirect administration. Indirect administration includes the act of prescribing a composition comprising a rAAV vehicle described herein. Direct administration includes administration to cells in vitro, administration to cells in vivo, administration to a patient by a medical professional or self-administration by the patient.

In embodiments, including both monotherapy with a composition described here and combination therapies with one or more additional therapies or therapeutic agents, the administration of a composition of the invention leads to the elimination of a symptom or complication of the disease being treated, however elimination of the disease is not required. In one embodiment, the severity of a symptom is decreased.

### EXAMPLE 1

### In Vitro Functional Assessment

A study was conducted to assess the in vitro functional efficacy of different rAAV expression plasmids. Transmembrane trafficking of the expressed transgene was assessed by immunocytochemistry. Electrophysiological recordings were used to assess function of the transgene. The following five constructs were tested:

Three constructions produced fusion proteins of the transgene with a fluorescent reporter protein, either GFP or tdTomato:
pCAG-ChRown-GFP-mWPRE-hGHpA
pCAG-ChRown-GFP-hGHpA
pChRown-tdTomato-hGHpA

Two of the constructs produced the transgene without fusion to the fluorescent protein:
pCAG-ChRown-mWPRE-hGHpA
pCAG-ChRown-hGHpA

HEK cell culture, DNA transfection and Patch Clamp Recordings: HEK293f cells were maintained in Advance Dulbecco's minimum essential medium (Life Technologies, Grand Island, NY, USA) supplemented with 5% fetal bovine serum, 1× minimum essential medium (MEM) non-essential amino acid solution, 100 U/mL penicillin G, and 100 mg/mL streptomycin at 37° C. in a humidified atmosphere of 95% air and 5% CO₂. HEK293f cells were passaged weekly.

For patch clamp recordings, HEK 293f cells were seeded onto 35 mm dishes. All-trans retinal (1 μM) was added to the culture media at the time of cell seeding. Cells were transfected with 1 μg plasmids using Lipofectamine 2000 (Life Technologies) and patch-clamp recordings were performed 2 days after transfection. Recordings in the whole-cell configuration were made using standard procedures at room temperature (22° C.). The extracellular recording solution contained the following (in mM): 138 NaCl, 1 NaHCO₃, 0.3 Na₂HPO₄, 5 KCl, 0.3 KH₂PO₄, 1.25 CaCl₂, 0.5 MgSO₄, 0.5 MgCl₂, 5 HEPES, 22.2 glucose, and 0.001% (v/v) phenol red, with the pH adjusted to 7.2 using 0.3 N NaOH. The extracellular solution is based on Normal Hank's solution with supplemental 1 mM all-trans retinal. The electrode solution contained the following (in mM): 110 Cs-Cl, 30 TEA-Cl, 2 MgCl₂, 0.1 CaCl₂, 10 EGTA, and 10 HEPES, with the pH adjusted to 7.25 using CsOH. The electrodes were pulled with vertical pipettes puller (Narishige, Japan), coated with silicone, and then polished with bulb filament (Narishige, Japan). Light stimuli were generated by 150-W xenon lamp-based scanning monochromators with a bandwidth of 10 nm (TILL Photonics, Germany). The light stimuli were coupled to the microscope with an optical fiber. The light stimulation of pulse was either 10 ms or 1 s. The light intensity without neutral density is 1.3×10¹⁷ photons/cm²s without neutral density filters. The light intensity was adjusted by using ND filters (ASI, Applied Scientific Instrumentation, Oregon, USA).

All data are expressed as the mean±SD, with “n” indicating the number of animals. The one-way analysis of variance (ANOVA) is used to determine whether there are statistically significant differences between groups.

Results: Fluorescent and DIC images of HEK cells after transfection with the transgene-fluorescent protein fusions indicated transmembrane expression was achieved. All five constructs produced light evoked currents in patch clamp assays. The off rates of the currents evoked by 10-ms light pulse from each of the five constructs were similar, all between 600-700 ms. Light-elicited currents in response to 1-s pulses of incrementally increasing light intensity resulted in current amplitudes and inactivation properties of the current. No appreciable differences in expression were observed with the transgene-GFP fusion construct comprising mWPRE versus the fusion construct lacking the mWPRE element.

### EXAMPLE 2

### Murine In Vivo Functional Assessment

In vivo functional assessment of test constructs was performed in a blind Opn4^{-/-}Gnat1^{-/-}Cnga3^{-/-} triple knockout (TKO) transgenic mouse line. TKO mice lack an optomotor response (OMR) making it an ideal model system for the assessment of the restoration of visual function using optomotor behavioral assays. Vector constructs were intravitreally injected into the eyes at a dose of 7.5E+9 vg per eye (n=6 per group).

Group designations and constructs:
R01: pCAG-ChRown-GFP-mWPRE-hGHpA
R02: pCAG-ChRown-GFP-hGHpA
R03: pCAG-ChRown-mWPRE-hGHpA
R04: pChRown-tdTomato-mWPRE-hGHpA
R05: pCAG-ChRown-hGHpA

Viral Vector Injection: Intravitreal injections of the viral vectors were administered to 1-3-month-old TKO mice. One hour before the injection experiment, the EthiqaXR (buprenorphine, 3.25 mg/kg) was intraperitoneal injected. Then, the animal was anesthetized with an intraperitoneal injection of a mixture of 100 mg/kg ketamine and 12 mg/kg xylazine. Viral vectors (1.5 μL) diluted in PBS at a titer of 5×10¹² vg/mL were intravitreally injected into both eyes of each animal. The viral vectors were injected with a programmable Nanoliter Injector III (Drummond Scientific, Broomall, PA, USA) using glass micropipettes. The enter site was ˜0.5 mm posterior to the sclera.

Optomotor Response: Optomotor response (OMR) tests were performed >1 month after viral injection. Two optomotor systems were used for the tests: a custom optomotor system (cOMS) (Pan lab, Detroit, USA) and a commercially purchased OptoDrum (StriaTech, Germany). The cOMS was used to measure the threshold light sensitivity that can evoke OMR, defined as light sensitivity.

For cOMS, the light stimulus is generated by blue LEDs with a peak wavelength of 470 nm mounted on the inner face of a wooden cylinder (40 cm diameter×51 cm height). The light intensity of the LEDs is controlled by a digital power supply with voltage output. The light intensity with the unit of μW/cm² at each of the voltage values was measured by a light meter at the center of the platform. The light intensity is converted to photons/cm²s, based on a formulate of 1 W/cm²=[wavelength/500]×2.5×10¹⁸ photons/cm²s. The highest light intensity that can be reached in cOMS was 1×10¹⁶ photons/cm²s.

A series of exchangeable drums (30 cm diameter×30 cm height) were made by marking strips on the wall of acrylic cylinders at spatial frequencies of 0.031, 0.042, 0.064, 0.092, 0.13, and 0.19 cycles per degree. The drums were covered with light-diffuser film. Rotation of the drum was controlled by a digital motor and set at 4 rpm or 24 degree per second. During testing, unrestrained animals were placed on a central platform (6.5-cm diameter) positioned 11.5 cm above the floor. A video camera was mounted above the apparatus for animal behavior monitoring and video capture.

To determine the threshold light intensity at each grating frequency, the ability to elicit head tracking in each animal in response to drum rotation was assessed starting at a relatively high light intensity. Head tracking was tested for both clockwise and counterclockwise directions. Once tracking was confirmed, the light intensity was systematically decreased to determine the lowest light intensity that was still be able to elicit head tracking.

Immunohistochemistry: Animals were euthanized by CO₂ asphyxiation followed by decapitation for immunohistochemical and western blot analysis. Mice eyes were enucleated and then fixed with 4% paraformaldehyde in 0.1M phosphate buffer (PB) at room temperature for 20 min. Immunofluorescence staining was examined in retinal whole mounts. The following primary antibodies were used in this study: Rabbit anti-ChRown (1:50; produced by Innovagen AB, SWEDEN), mouse anti-GFP (1:1000; Neuromab, UC Davis, Davis, CA, USA), and rabbit anti-RBPMS (1:1,000; ABN1362, Millipore Sigma, Temecula, CA, USA). The secondary antibodies were conjugated to Alexa 488 (1:600), Alexa 555 (1:1,000), or Alexa 594 (1:500; Thermo Fisher Scientific, Waltham, MA, USA). Fluorescence images were obtained using a ZEISS APOTOME2 Optical Photomicroscope (Apotome; Carl Zeiss Microscopy GmbH, Jena, Germany). The brightness and the contrast were adjusted using the ZEN3.5 software. RGC counting was performed using stacked images generated in ZEN3.5-3D mode.

All data are expressed as the mean±SD, with “n” indicating the number of animals. The one-way analysis of variance (ANOVA) is used to determine whether there are statistically significant differences between groups.

Safety/toxicity: There were no in life safety observations or animal deaths. There were no signs of retinal toxicity on histological analysis.

Light sensitivity: The threshold light intensities that are required to elicit OMR at the optimal frequency of 0.042 cycles/degree, defined as light sensitivity, are shown in Table 1. The highest light sensitivities were observed in treatment groups R01 and R03.

Unexpectedly, treatment groups R01 and R03 in particular exhibited higher light sensitivity compared to constructs lacking the mWPRE element, R02 and R05. This was unexpected at least based on the relative expression of the constructs with and without the mWPRE element, which as discussed above was not appreciably different. This indicates that expression, although related to function, is not the only factor influencing transgene function. Other factors include appropriate expression within the cell, in this case in the cell membrane as opposed to intracellular expression, as well as correct protein folding and orientation in the membrane.

Group R03, which was treated with a construct containing ChRown but not GFP, showed similar light sensitivity compared to group R01, where the transgene was a ChRown-GFP fusion protein. The similar function observed for treatment groups R01 and R03 was unexpected from prior publications, e.g., by Gauvain, G. et al. Optogenetic therapy: high spatiotemporal resolution and pattern discrimination compatible with vision restoration in non-human primates. Commun. Biol. 4:125 (2021); and Sahel et al. Partial recovery of visual function in a blind patient after optogenetic therapy, Nature Medicine 27:1223-1229 (2021), which indicated that the transgene should be stabilized by fusion with a fluorescent reporter. Such increased stabilization was expected to result in improved function, which here should have resulted in higher light sensitivity for treatment group R01. Further unexpectedly, treatment group R04, in which the transgene was a fusion protein of ChRown with another fluorescent reporter, tdTomato, resulted in worse light sensitivity compared to ChRown alone. Fusion proteins with tdTomato in particular had been reported by Guavain and Sahel to stabilize the therapeutic transgene. These results highlight the difficulties in predicting the function of a particular transgene construct based on prior studies using the same or similar genetic elements.

TABLE 1
Light sensitivity thresholds in treated TKO mice

| Viral vector treated | Light sensitivity | Animal |
|---|---|---|
| Mouse group | (photos/cm²s; mean ± SD) | number |
| R01 | 1.74 ± 0.52 × 10¹³ | 6 |
| R02 | 6.33 ± 4.42 × 10¹³ | 6 |
| R03 | 2.69 ± 0.97 × 10¹³ | 6 |
| R04 | 4.00 ± 1.78 × 10¹³ | 6 |
| R05 | 1.71 ± 0.873 × 10¹⁴ | 6 |

Immunohistochemistry: The TKO mice were euthanized, and the retinas were isolated for immunostaining. R01-R04 treated retinas were labeled with anti-GFP antibody, anti-mCherry antibody or anti-ChRown (275-288) antibody. The retinas of R01-R05 treated retinas were also labeled with anti-RBPMS antibody, a ganglion cell specific marker. As a note, the use of RBPMS antibody is preferred to DAPI to label RGC nuclei because DAPI can also label the nucleus of microglia and displaced amacrine cells. Robust expression of both the ChRown protein and the fusion proteins, ChRown-GFP and ChRown-tdTomato, was observed in inner retinal neurons, predominantly in RGCs. In this study, GFP was observed on cell membranes without evidence of intracellular aggregation, although protein misfolding and aggregation remain of concern with transgene-FP fusion proteins. Importantly, the ChRown protein itself, not in the form of a fusion protein, was properly expressed on cell membranes of ganglion cell soma and dendrites as well as in some horizontal cells and amacrine cells.

### EXAMPLE 3

### Ex-Vivo Multi-Electrode Array Analysis

Explants from TKO mice treated with pCAG-ChRown-GFP-mWPRE-hGHpA (R01) and pCAG-ChRown-mWPRE-hGHpA (R03) were evaluated for retinal electrophysiological responses using multi-electrode array analysis.

Multi-electrode array (MEA) recordings: MEA recordings were performed using previously described procedures in Bi, A., et al., Ectopic expression of a microbial-type rhodopsin restores visual responses in mice with photoreceptor degeneration. Neuron, 2006. 50 (1): p. 23-33. Animals were euthanized by decapitation. The retina was dissected and placed on a piece of nitrocellulose filter paper with the photoreceptor side down (Millipore, Bedford, MA, USA). The mounted retina was placed in the MEA-64 MEA recording chamber comprising 30-mm-diameter electrodes spaced 200 mm apart (Multi Channel System MCS, Reutlingen, Germany), with the ganglion cell layer facing the recording electrodes. The retina was continuously perfused with an oxygenated extracellular solution at 34° C. during all experiments. The extracellular solution contained the following (in mM): 124 NaCl, 2.5 KCl, 2 CaCl₂, 2 MgCl₂, 1.25 NaH₂PO4, 26 NaHCO₃, and 22 glucose (pH 7.35) with 95% O₂ and 5% CO₂. The extracellular solution is freshly made every time before the recording and bubbled with CO₂ 30 minutes before tissue dissection. The interval between the onset of each light stimulus was 20s. Signals were filtered between 200 Hz (low cutoff) and 20 kHz (high cutoff). A threshold of 0.4% was used to detect action potentials, and action potentials from individual neurons were determined with a standard expectation-maximization algorithm using offline Sorter software (Plexon, Dallas, TX, USA) to run T-Dist E-M Sort. The results were plotted using NeuroExplorer software (Nex Technologies, Madison, AL, USA). The relationship between the response-amplitude attenuation and frequency was measured from the averaged spike rate histograms.

Light stimuli were generated using 150W xenon lamp-based scanning monochromators with a bandwidth of 10 nm (TILL Photonics, Germany). The light intensity was adjusted by using neutral ND filter device (ASI, Applied Scientific Instrumentation, Oregon, USA) with software programmed control. The light stimuli were directly projected to the bottom of the recording chamber through an optical fiber.

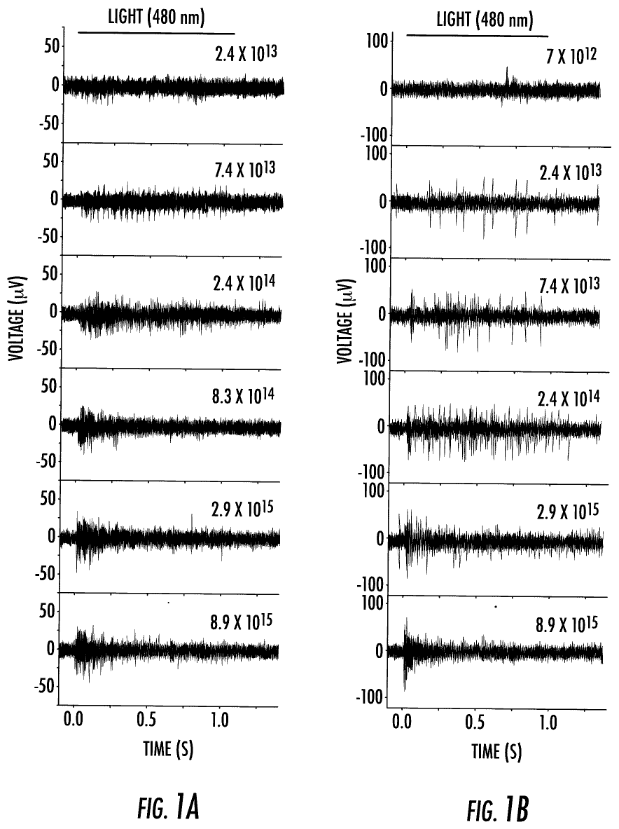

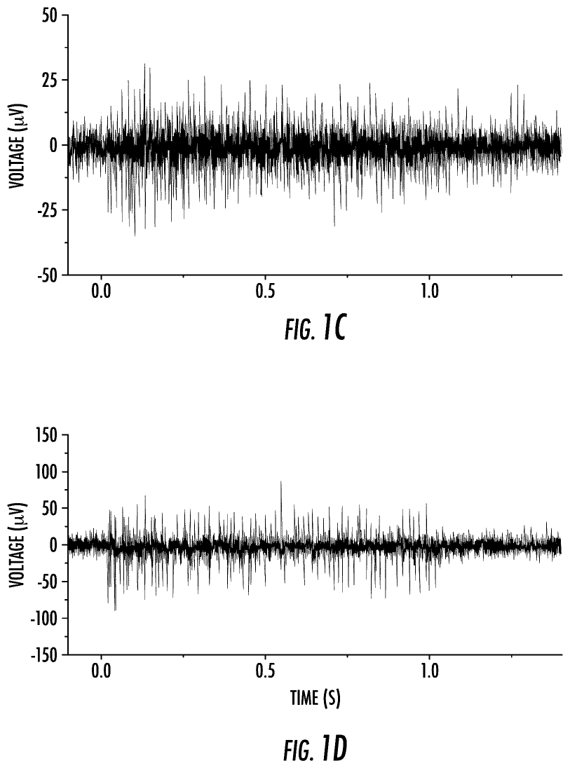

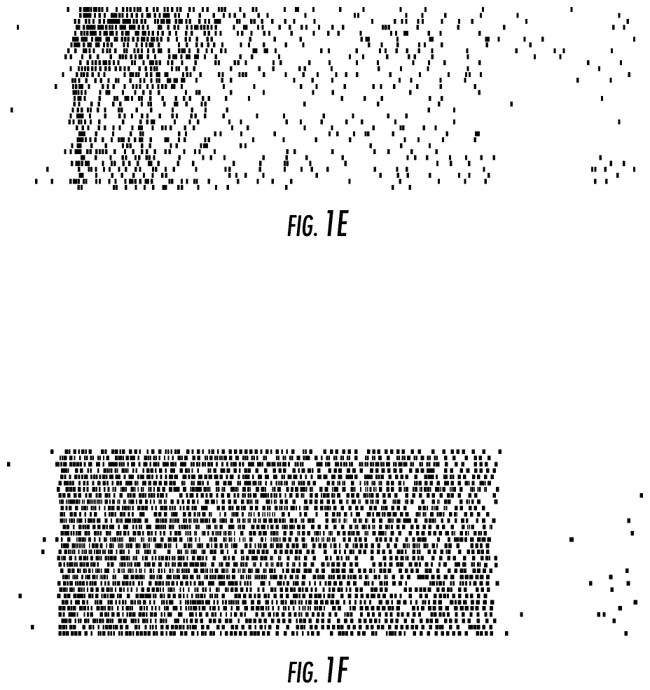

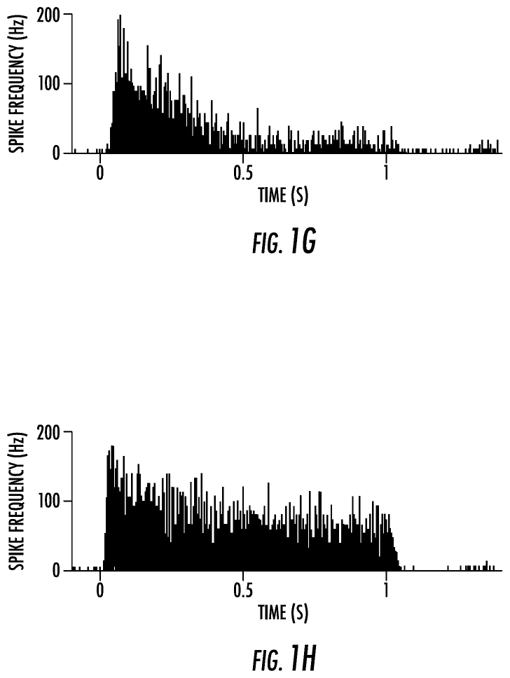

Results: Light dependent transgene-mediated spiking activities of RGCs were observed for both constructs in the MEA recordings from retinal whole mounts. FIG. 1A shows ChRown mediated spiking activities; FIG. 1B shows activity for the fusion protein, ChRown-GFP. Light intensities measured as photons/cm²s are shown in the upper right of each trace. The threshold light sensitivity required to elicit spiking activities was a low 10¹³ photons/cm²s. FIG. 1C and FIG. 1D show the light-evoked spikes recorded from a single electrode for ChRown and ChRown-GFP, respectively. FIG. 1E and FIG. 1F show raster plots of 30 consecutive light-elicited spikes originated from a single neuron (30 trials and 10 min recordings) for ChRown and ChRown-GFP, respectively. FIG. 1G and FIG. 1H show the averaged spike rate histograms for ChRown and ChRown-GFP, respectively. Transgene-mediated spiking activities of RGCs were stable during repeated light stimulation.

### EXAMPLE 4

### Murine Pharmacology

The OptoDrum system was used to measure visual acuity in animals treated with a low or high dose of the following test articles (treatment group in parenthesis):
pCAG-ChRown-GFP-mWPRE-hGHpA (R01)
pCAG-ChRown-mWPRE-hGHpA (R03)
pCAG-GFP (R00, negative control)

For OptoDrum, a virtual drum with different spatial frequencies is generated by four panel of 24″ LCD monitors \. The detection of animal head tracking was based on an automatic program. During testing, the spatial frequency was systematically increased. Visual acuity is defined by the highest grating frequency that is able to elicit the head tracking.

TABLE 2
Animal Assignments

| | Species/ | Test | | | | In life |
|---|---|---|---|---|---|---|
| Group | model | article | n | IVT | Dose | period |
| 1 | TKO mouse | 7m8.CAG- | 6 | Both | 7.5E+9 vg | 2 months |
| | | GFP-mWPRE | | eyes | (high) | |
| 2 | TKO mouse | R01 | 6 | Both | 7.5E+8 vg | 2 months |
| | | | | eyes | (low) | |
| 3 | TKO mouse | R03 | 6 | Both | 7.5E+8 vg | 2 months |
| | | | | eyes | (low) | |
| 4 | TKO mouse | R01 | 6 | Both | 7.5E+9 vg | 2 months |
| | | | | eyes | (high) | |
| 5 | TKO mouse | R03 | 6 | Both | 7.5E9 vg | 2 months |
| | | | | eyes | (high) | |

Viral Vector Injection: Intravitreal injections of the viral vectors were administered as follows. One hour before the injection experiment, the EthiqaXR (buprenorphine, 3.25 mg/kg) was intraperitoneal injected. Then, the animal was anesthetized with an intraperitoneal injection of a mixture of 100 mg/kg ketamine and 12 mg/kg xylazine. Viral vectors (1.5 μL) diluted in PBS at a titer of 5×10¹² vg/mL were intravitreally injected into both eyes of each animal. The viral vectors were injected with a programmable Nanoliter Injector III (Drummond Scientific, Broomall, PA, USA) using glass micropipettes. The enter site was ˜0.5 mm posterior to the sclera.

All data are expressed as the mean±SD, with “n” indicating the number of animals. The one-way analysis of variance (ANOVA) is used to determine whether there are statistically significant differences between groups.

There were no in life safety observations or animal deaths.

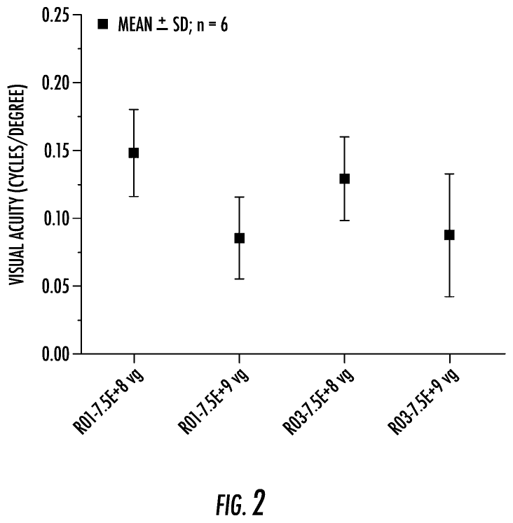

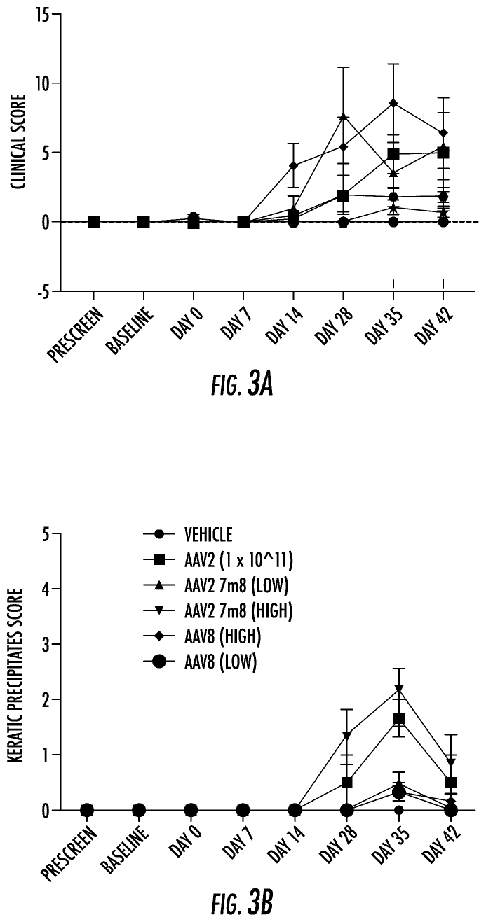

Light sensitivity: Thresholds were measured at the grating frequency at 0.042 cycle/degree. Results for the two doses in treatments groups 1 and 3 are shown in FIG. 2. No responses were elicited in animals that received R00 (negative control). There was no statistically significant difference in the two tested doses.

A further study was performed in the TKO mouse model (group sizes 3-4 animals) to assess light sensitivity behavioral responses across a range of doses from approximately 1×10⁷ vg/eye to 1×10¹¹ vg/eye. Unexpectedly, given the complex biology of optogenetically restored vision, the dose response curve followed a smooth exponential type function. Furthermore, the data reveal that the minimum dose at which the maximum light sensitivity is observed is approximately at dose of 1E8 vg which is when >50% of retinal ganglion cells can be shown histologically to be expressing the ChRown protein. The human equivalent dose (HED) of 1E8 vg/eye (mouse) is 2-6E9 vg/eye (human).

### EXAMPLE 5

### NHP Study

A study was conducted in non-human primates (NHP) to compare the ocular biodistribution and GFP expression of three different AAV capsids (AAV2, AAV8 and AAV2-7m8) following intravitreal (IVT) administration to African green monkeys. For this study the GFP transgene was fused to ChRown.

### Methods

The five test articles each contained the same transgene expression construct encapsulated within either an AAV2 (wt), AAV8 (wt), or AAV2 (7M8) viral capsid. Two viral titers were tested, 2×10¹² vg/mL and 2×10¹³ vg/mL as shown in Table 3. The transgene expression construct was pCAG-ChRown-GFP-mWPRE-hGHpA.

The control article was Vehicle (1×PBS+0.001% Pluronic F-68).

Monkeys underwent baseline screening to assess AAV neutralizing antibody (Nab) seronegativity, complete blood count (CBC), general well-being, and ocular health by slit lamp biomicroscopy, fundoscopy, color fundus imaging, confocal scanning laser ophthalmoscopy (cSLO), and optical coherence tomography (OCT). Nab-negative monkeys with >50% transduction at <1:10 dilution with normal findings were enrolled in the study and assigned to treatment groups (Table 3). For baseline screening and all subsequent procedures (Table 4), anesthesia was achieved with intramuscular ketamine (8 mg/kg) and xylazine (1.6 mg/kg) to effect, and pupil dilation with topical 10% phenylephrine, 1% tropicamide and/or 1% cyclopentolate.

TABLE 3
Treatment Groups

| | | Concentration | Volume | Dose |
|---|---|---|---|---|
| Group | Treatment | (vg/ml) | (ul/eye) | (vg/eye) |
| 1 | vehicle | — | 50 | |
| 2 | AAV2 wt | 2 × 10¹² | 50 | 1 × 10¹¹ |
| 3 | AAV2 7M8 | 2 × 10¹² | 50 | 1 × 10¹¹ |
| 4 | AAV2 7M8 | 2 × 10¹³ | 50 | 1 × 10¹² |
| | high | | | |
| 5 | AAV8 | 2 × 10¹² | 50 | 1 × 10¹¹ |
| 6 | AAV8 high | 2 × 10¹³ | 50 | 1 × 10¹² |

TABLE 4
Study Schedule

| Event | # | eye | S* | B | 0 | 1 | 2 | 4 | 5 | 6 | 6 |
|---|---|---|---|---|---|---|---|---|---|---|---|
| dosing | 16 | OU | — | — | X | — | — | — | — | — | — |
| MP** | 16 | — | — | — | X | X | X | X | — | X | — |
| wt | 16 | — | X | X | X | X | X | X | — | X | — |
| exam | 16 | — | X | — | — | — | — | — | — | — | — |
| T | 16 | OU | — | X | — | X | X | X | — | X | — |
| SL | 16 | OU | X | — | X+ | X | X | X | X | X | — |
| C | 16 | OU | — | X | — | — | X | X | — | X | — |
| F | 16 | OU | — | X | — | — | X | X | — | X | — |
| OCT | 16 | OU | — | X | — | — | X | X | — | X | — |
| cSLO | 16 | OU | — | X | — | — | X | X | — | X | — |
| Nab | 16 | OU | X | — | — | — | — | — | — | X | — |
| CBC | 16 | OU | X | X | — | — | — | — | — | X | — |
| Necr | 16 | OU | — | — | — | — | — | — | — | — | X |

In Table 4, numbers in first row are time in weeks, corresponding days are 0, 7, 14, 28, 35, 42, 43; dosing was by intravitreal injection to both eyes (OU); X indicates event occurring in all groups; * 32 animals were screened for Nabs and CBC at week 3; B=baseline, day 0 week 0; ** MP (methylprednisolone 8 mg/kg IM) administered weekly; wt=body weight; exam=physical exam; T=tonometry; SL=slit lamp exam; +Slit lamp exams were performed immediately after dosing; C=color fundus imaging; F=fluorescence imaging; OCT=optical coherence tomography; cSLO=confocal scanning laser ophthalmoscopy; Nab=neutralizing antibody; CBC=complete blood counts; Necr=necropsy.

Animals were anesthetized with ketamine/xylazine (8.0 mg/kg ketamine [Covetrus]/1.6 mg/kg xylazine [Covetrus]) in a sterile, mixed cocktail administered to effect for baseline screening and all non-surgical procedures. General well being was assessed before, during and after sedation. Temperature within the non-air-conditioned ventilated study enclosure ranged from 22.3-33.0° C. over the duration of the study. Humidity ranged from 64.0-100.0%.

Topical proparacaine 0.5% was administered, an eye speculum placed, then the ocular surface rinsed with 5% Betadine solution followed by a sterile 0.9% saline rinse. Intravitreal (IVT) injections were performed in both eyes (OU) according to the treatment assignment using a 31-gauge 0.375-inch needle inserted inferotemporally at the level of the ora serrata ˜2 mm posterior to the limbus. Following injection, a topical neomycin, polymyxin, bacitracin antibiotic ophthalmic ointment was administered.

Animals received prophylactic corticosteroid treatment with a taper. Topical proparacaine 0.5% was administered, an eye speculum placed, then the ocular surface rinsed with 5% Betadine solution followed by a sterile 0.9% saline rinse. Intravitreal (IVT) injections were performed in both eyes (OU) according to the treatment assignment using a 31-gauge 0.375-inch needle inserted inferotemporally at the level of the ora serrata ˜2 mm posterior to the limbus. Following injection, a topical neomycin, polymyxin, bacitracin antibiotic ophthalmic ointment was administered.

Animals received intramuscular (IM) delivery of methylprednisolone (8 mg/kg) on Day 0, then weekly for the duration of the in-life portion of the study (six weeks). Monkeys received steroids pro re nata (PRN) in accordance with the following criteria guided by slit lamp exams scheduled on the basis of individual animal clinical findings:
1. If aqueous cell (AC)+1 or less was observed, no treatment was administered.
2. If AC was 2+ or greater, subconjunctival dexamethasone (100 μL of 10 mg/mL) was administered and eyes were evaluated after 48-72 hours, repeating subconjunctival dexamethasone if AC decreased (2 or below) and eyes were observed 7 days later.
3. If treated as above and AC of greater than 3 or more persisted one week after subconjunctival dexamethasone, IVT triamcinolone (TMC) (100 μL of 40 mg/mL) was administered and eyes were evaluated within 48-72 hours.

At any time point, if AC or VC (vitreous cell) is 4+, eyes were treated with IVT TMC (100 μL of 40 mg/mL) and evaluated in 48-72 hours.

At designated time points during the study, various clinical observations were made and scored using a nonhuman primate ophthalmic scoring system and summary score derived from exam components. Clinical observations included intraocular pressure (IOP) measurements, eye examinations by slit lamp biomicroscopy, optical coherence tomography (OCT), and confocal scanning laser ophthalmoscopy (cSLO). In addition, bilateral color anterior and fundus images and fluorescent fundus images to detect GFP expression were captured. Fluorescence photographs were evaluated using a scoring system to define extent of GFP expression with quantitative analysis applied as appropriate where a score of 0=absent; 1=trace; 2=slight; 3=moderate; 4=bright and 5=intense in the foveal, peripheral and perivascular regions of the eye. Other periodic clinical observations included respiratory rate, heart rate, body temperature, thoracic auscultation, integrity of the integument and body weight. General well being was confirmed twice daily. Animals were evaluated for signs of ocular inflammation, including swelling, discoloration, squinting, and eye rubbing.

At designated time points, intraocular pressure (IOP) measurements were collected using a TonoVet (Icare®, Finland) tonometer set to the dog (d) calibration setting. Three measures were taken from each eye at each time point and the mean IOP defined.

At designated time points, both eyes (OU) were examined by slit lamp biomicroscopy. Scoring was applied to qualitative clinical ophthalmic findings using a nonhuman primate ophthalmic scoring system and summary score derived from exam components.

Slit lamp examinations for animals in Cohort 2 were performed on Day 8 rather than Day 7 and slit lamp examinations for animals in Cohort 1 were performed on Day 15 rather than Day 14. This had no impact on the study.

At designated time points, bilateral color anterior and fundus images and fluorescent fundus images to detect GFP expression were captured with 50° of view centered on the fovea using a Topcon TRC-50EX retinal camera with Canon 6D digital imaging hardware and New Vision Fundus Image Analysis System software. Images were obtained centered on the fovea, with four additional image series obtained of the superior, inferior, nasal, and temporal quadrants. Color fundus photos were captured with shutter speed (Tv) 1/25 sec, ISO 400 and flash 18. Monochromatic and color fluorescent images were captured with exciter and barrier filters engaged (480 nm exciter/525 nm barrier filter), Tv ⅕ sec, ISO 3200 and flash 300.

At designated time points OCT and cSLO were performed OU using a Heidelberg Spectralis OCT HRA (or OCT Plus), employing the Heyex TruTrack and AutoRescan follow-up imaging function referencing the baseline images. cSLO infrared (IR) and autofluorescence (AF) retinal images were obtained using the 50° lens, with images obtained centered on the fovea, as well as additional images of the superior, inferior, nasal, and temporal quadrants. IR and AF images were followed by an overall OCT volume scan of the entire macula at a dense scan interval. Images were qualitatively evaluated with quantitative analysis of the OCT retinal thickness data and GFP expression, as appropriate.

General well being was confirmed twice daily by cage side observations beginning one week prior to dosing. Food consumption and overall appetite was assessed by visual inspection of the feed pan and cage floor prior to cage washing before daily feeding. Animals were evaluated for signs of ocular inflammation, including swelling, discoloration, squinting, and eye rubbing.

Body weights were obtained at designated time points.

Whole blood (3 mL) was collected via the femoral or saphenous vein at designated time points. Blood was transferred to vacutainer tubes (in the absence of anticoagulant) and incubated at room temperature for approximately 1 hour before centrifugation 4000 rpm for 10 minutes at 4° C. and transfer of serum aliquots (˜0.5 mL×2 aliquots per time point) to Sarstedt cryotubes. Aliquots were stored and shipped below −70° C. to a Sponsor-designated laboratory for Nab analysis.

At designated time points 0.5 mL blood was transferred directly to K₃EDTA lavender top vacutainer tubes (Greiner MiniCollect EDTA tubes REF #450475) and maintained on ice until CBC with differentials analysis on a Hemavet analyzer.

After confirming final image quality prior to the defined terminus, monkeys were sedated intramuscularly with ketamine (8 mg/kg) and xylazine (1.6 mg/kg) to effect and euthanized with sodium pentobarbital (100 mg/kg IV).

Both globes (OU) were enucleated and excess orbital tissue was trimmed with optic nerve attached and the globe then placed in 4% paraformaldehyde in 0.1 M phosphate buffer saline (PBS) for 24 hours at room temperature, then the anterior segment was removed and the eye cup flat mounted and the retina collected and suspended in PBS with 0.05% azide within a 60 mL vial (completely filled) and shipped to a Sponsor-designated laboratory in a Credo Cube temperature-controlled container design to maintain 4° C.

A necropsy was performed, and additional tissues optic nerve OU, optic chiasm, lateral geniculate nucleus (right and left) and visual cortex (right and left) were collected for possible histopathology and GFP biodistribution analysis, pending in-life and ocular tissue findings. Samples were transferred to 10% NBF in labelled vials and maintained at the testing facility per Sponsor request.

Data generated from protocol-defined endpoints was collated, summarized, and analyzed as descriptive statistics.

Retinal biodistribution and transgene expression was assessed by repeat in vivo qualitative retinal fluorescence imaging. After confirming final image quality prior to the defined terminus, monkeys were sedated and euthanized.

Both globes (OU) were enucleated, excess orbital tissue was trimmed with optic nerve attached and the globe was fixed in paraformaldehyde prior to removal of the anterior segment mounting of the eye cup. The retina was collected for immunohistochemistry. Additional tissues were collected including optic nerve OU, optic chiasm, lateral geniculate nucleus (right and left) and visual cortex (right and left).

### Results

Test articles were administered in accordance with treatment assignment without complication. In all cases, administration was by intravitreal injection. There was one animal per group and for each animal, a dose of 1×10¹¹ vg/eye was administered to the right eye (OD) and a dose of 1×10¹² vg/eye was administered to the left eye (OS), except for Groups 14-16 in which the dose in both eyes was 1×10¹¹ vg/eye.

TABLE 5
Test and Control Article Administration

| Grp | Treatment, OD | Treatment, OS |
|---|---|---|
| 1 | vehicle | vehicle |
| 2 | AAV2-ChRown-GFP | AAV2 7M8-ChRown-GFP |
| 3 | AAV2-ChRown-GFP | AAV2 7M8-ChRown-GFP |
| 4 | AAV2-ChRown-GFP | AAV2 7M8-ChRown-GFP |
| 5 | AAV2-ChRown-GFP | AAV2 7M8-ChRown-GFP |
| 6 | AAV2- ChRown-GFP | AAV2 7M8-ChRown-GFP |
| 7 | AAV2-ChRown-GFP | AAV2 7M8-ChRown-GFP |
| 8 | AAV2 7M8-ChRown-GFP | AAV8-ChRown-GFP |
| 9 | AAV2 7M8-ChRown-GFP | AAV8- ChRown-GFP |
| 10 | AAV2 7M8- ChRown-GFP | AAV8- ChRown-GFP |
| 11 | AAV2 7M8- ChRown-GFP | AAV8- ChRown-GFP |
| 12 | AAV2 7M8- ChRown-GFP | AAV8- ChRown-GFP |
| 13 | AAV2 7M8- ChRown-GFP | AAV8- ChRown-GFP |
| 14 | AAV8- ChRown-GFP | AAV8- ChRown-GFP |
| 15 | AAV8- ChRown-GFP | AAV8- ChRown-GFP |
| 16 | AAV8- ChRown-GFP | AAV8- ChRown-GFP |

Mean intraocular pressure (IOP) was measured at designated time points from Baseline to Day 42. IOP was within normal range for eyes within all treatment groups over the course of the study. There were no dose-related findings.

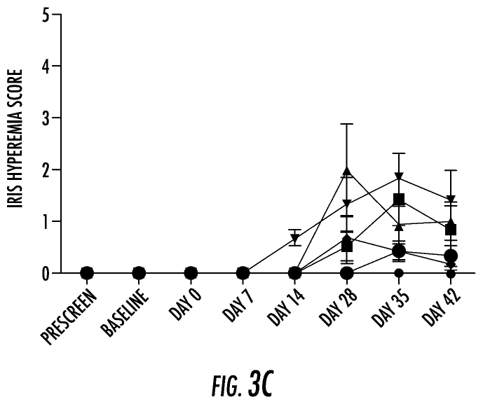

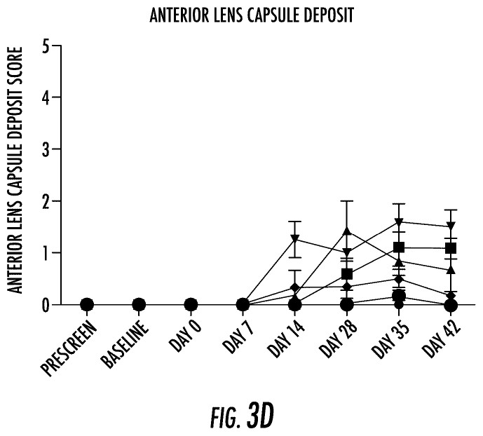

There were no slit lamp abnormalities detected except for intraocular inflammation commencing around day 14. The presence or absence of intraocular inflammation was assessed by slit lamp biomicroscopy in all eyes at designated time points and ranged from absent to mild (FIG. 3A-FIG. 3D). There was no evidence of retinal or other ocular pathology. Inflammation was noted in the in the AAV2 treatment group, the AAV2 7m8 low dose treatment group, and in the AAV2 7m8 high dose treatment group. Inflammation was absent in the vehicle treatment group and was minimal in the AAV8 low dose and AAV8 high dose treatment groups. While there was variability between animals, it appeared that the level of inflammation was related to the degree of retinal GFP fluorescence.

Per protocol, at day 21, animals D469, D655, D656, D467 and O9922 received IVT triamcinolone (100 μL of 40 mg/mL) and eyes were evaluated within 48-72 hours. On Day 28, O9988, S1071, O9922, DO15 and D655 received IVT triamcinolone (100 μL of 40 mg/mL) and eyes were evaluated within 48-72 hours. Animal D655 exhibited the most severe inflammation at day 21 and additional meloxicam was administered subcutaneously (0.2 mg/kg) for analgesia for 5 days.

After treatment with triamcinolone, eyes exhibited complete resolution of inflammation by days 35 & 42.

At designated time points over the course of the study, bilateral color anterior and fundus images and GFP fluorescence fundus images were obtained to assess ocular health and GFP expression. Retina morphology remained within normal limits. There were no signs of retinal detachment, edema, vasculitis, or other major ocular issues observed in the fundus images.

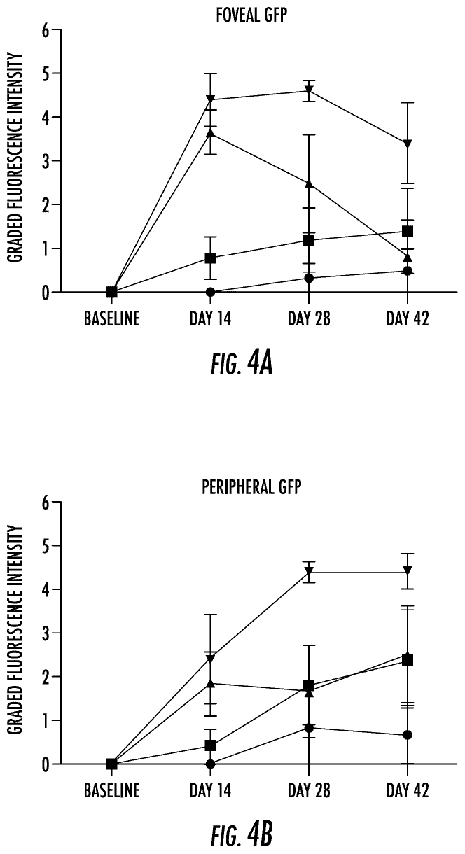

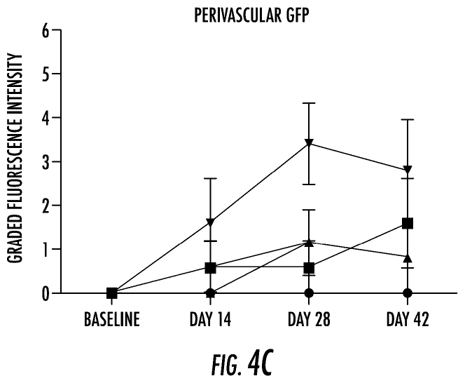

GFP fluorescence signal scoring on a scale of 0-5 are presented in FIG. 4A-FIG. 4C. No GFP was observed in eyes treated with the Vehicle and eyes treated with AAV8 low dose. GFP expression in eyes treated with AAV2 7m8 high dose ranged from moderate to bright. Moderate GFP expression was observed in eyes treated with AAV2 7m8 low dose while GFP expression in eyes treated with AAV8 high dose and eyes treated with AAV2 was mostly trace to slight.

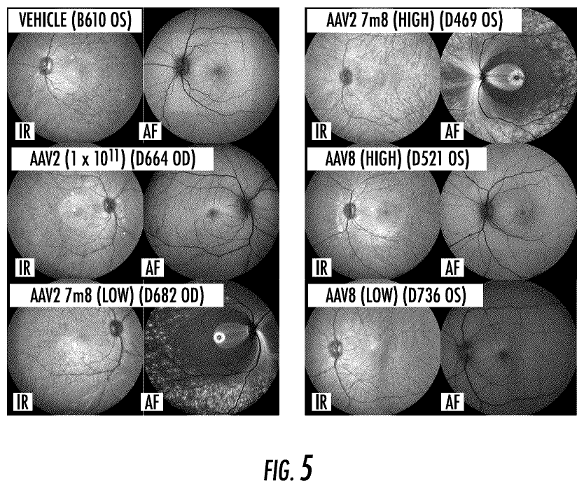

cSLO infrared (IR) and autofluorescence (AF) retinal images at at 50° Field of view were generated throughout the study at the designated time points. Representative Day 42 images from each treatment group are shown in FIG. 5.

Optical Coherence Tomography (OCT) was performed to assess retinal structure, thickness, and volume. Retinal thickness and volume remained stable for all treatment groups over the course of the study. Overall, average retinal thickness and volume was lowest in the AAV8 low dose and the AAV2 treatment groups compared to the other treatment groups. None of the treatment groups experienced fluctuations in retinal thickness and volume levels from Baseline to Day 42.

At Baseline respiratory rate, heart rate, body temperature, thoracic auscultation and integrity of the integument were assessed. Most animals in the study had a higher respiratory rate at baseline than the normal range (16-28 bpm for males and females). The animals that did display a higher respiratory rate all auscultated normally. It was determined by the veterinarian that these elevations were most likely due to an elevated respiratory rate prior to sedation and not a clinical concern. Heart rate was within normal range (76-120 bpm for males, 80-140 bpm for females) at baseline for all animals except for D699, which had a heart rate of 64 bpm. It was determined by the veterinarian that this animal's lower heart rate was likely due to a deeper sedation and not of clinical concern. Body temperature was within normal range (98-101.5° F. for males and females) for most animals at baseline, except for D699 (95.4° F.), S1071 (101.8° F.).

Twice daily general observations and did not reveal any clinical signs indicative of adverse systemic effects caused by the test articles. All animals were found to be in good general health throughout the study.

Body weights were obtained at designated time points. No major changes in individual animal body weights were observed over the course of the study.

Blood was collected, processed, and shipped to the Sponsor designated laboratory for Nab analysis.

Hematology assessments were performed at designated time points to assess general animal health. All animals recruited to the study were determined to be healthy by CBC parameters. At Baseline, animal D699 had a low hematocrit count and a high mean corpuscular hemoglobin count but returned to normal by Day 42. At Day 42, animal DO15 had a low platelet count and a low procalcitonin level.

Animals were euthanized at the study terminus and the designated ocular and non-ocular tissue samples were successfully collected and processed as needed for biodistribution and transgene expression analysis

### Conclusions

This study compared the retinal biodistribution and GFP expression levels of different AAV capsids following intravitreal (IVT) administration in African green monkeys. Achieved transgene expression was greatest in the AAV2 7m8 low dose (1×10¹¹ vg/eye) and in eyes treated with AAV2 7m8 high dose (1×10¹² vg/eye) as evaluated by qualitative observation and semi-quantitative scoring of GFP expression.

Intraocular inflammation, without evidence of other ocular pathology, was noted in most animals and was responsive to additional local steroid medication and showed resolution by the end of the study. Intraocular inflammatory responses are frequently seen in primates after intravitreal AAV. The etiology is not known; however, there is substantial inter-animal variability, dose dependence, and in this study appeared to relate also to the amount of retinal GFP expression.

At the low dose of 1×10¹¹ vg/eye, vectors comprising the variant VP1 capsid protein 7M8 showed unexpectedly high levels of transduction and expression in NHP retinal ganglion cells. Indeed, this appeared to be a ceiling effect as at 1×10¹¹ vg/eye, transduction and expression was not significantly higher.

This was unexpected for at least two reasons. First, the variant VP1 capsid protein, 7m8, was discovered using directed evolution in the mouse. It was not predictable that a capsid protein evolved for murine retinal transduction would be able to efficiently transduce retinal cells in the primate eye due to the structural differences between the murine and primate eye. The primate eye has major structural differences compared to the murine eye including a significant inner limiting membrane which is known to bind AAV serotypes, including AAV2, and prevent transduction of retinal cells. It was therefore surprising that the 7M8 capsid was able to transduce retinal cells of the primate eye with high efficiency, as shown here.

Second, the transgene here was a transmembrane protein fused to a fluorescent reporter, GFP. We expected that reduced protein folding, protein trafficking, and transmembrane localization would limit the fluorescence signal. To the contrary, we found abundant fluorescent reporter signal, suggesting a high level of transgene expression in the primate retinal cells. Given the potential for the fluorescent reporter to interfere with transgene expression, we hypothesized that even higher transgene expression could be achieved without fusion to a fluorescent reporter. In fact, this was the case.

### EXAMPLE 6

### NHP Study 2

Quantification of ChRown mRNA in Non-Human Primate (African Green Monkey) Tissues

The following describes quantification of transgene expression in tissue samples taken from rhesus monkeys administered a low, medium or high dose of rAAV particles. The viral titers were tested were 3×10¹⁰ vg/eye, 1×10¹¹ vg/eye and 2.35×10¹¹ vg/eye. The transgene expression construct was pCAG-ChRown-mWPRE-hGHpA. These assays were conducted in support of a clinical study. Measurement of ChRown RNA was determined using an NHP tissue RNA extraction procedure and RT quantitative polymerase chain reaction (RT-qPCR) assay.

The study included 34 NHP (20 male and 14 females) divided into five groups. These five groups were divided based on the dosage of drug product and tissue collected for biodistribution. Only Group 5 OU were collected for biodistribution analyses which had NHPs injected with low, medium, and high dosage of the drug product in the right eye (OD) only. RT-qPCR assay was performed with samples from Group 2 to 4 (6 male and 3 female AGMs per time point) from which 28 and 30 different systemic tissues were collected respectively to study toxicity and biodistribution of ChRown. While Group 5 included 3 male and 3 female AGMs (per time point) from which 45 tissues (32 Ocular-16 from each eye and 13 Systemic tissues) were collected respectively to study toxicity and biodistribution of ChRown. Two time point (Week 13 and week 25) samples were collected for each group.

Tissues assayed included: Eye (Aqueous humor, Vitreous humor, Optic nerve, Iris/ciliary body, Retina—macular, Retina—superior, Retina—nasal, Retina—inferior, Retina—temporal, RPE/choroid—macula, RPE/choroid—superior, RPE/choroid—nasal, RPE/choroid—inferior, RPE/choroid—temporal, Sclera), Brain (optic chiasm, optic tract-left, lateral geniculate nucleus-left, visual cortex-left, frontal cortex, temporal cortex, hindbrain), Dorsal root ganglion (Cervical-C3, Thoracic-T3, Lumbar-L3), Gross lesions, Heart, Kidney (left), Liver, Lung, Lymph node (mandibular and mesenteric), Ovary (left and right), Pancreas, Sciatic Nerve, Skeletal muscle, Small intestine, Spinal cord (cervical, thoracic and lumbar), Spleen, Stomach and Testis (left).

TABLE 6
Treatment Groups. OD refers to right eye

| Group | N | Eye | Treatment (vg/eye) | Terminus |
|---|---|---|---|---|
| 1A | 1F/1M | OD | vehicle (0) | week 13 |
| 1B | 1F/1M | OD | vehicle (0) | week 25 |
| 2A | 1F/2M | OD | low (3 × 10¹⁰) | week 13 |
| 2B | 1F/2M | OD | low (3 × 10¹⁰) | week 25 |
| 3A | 1F/2M | OD | medium (1 × 10¹¹) | week 13 |
| 3B | 1F/2M | OD | medium (1 × 10¹¹) | week 25 |
| 4A | 1F/2M | OD | high (2.35 × 10¹¹) | week 13 |
| 4B | 1F/2M | OD | high (2.35 × 10¹¹) | week 25 |
| 5A | 1F/1M | OD | low (3 × 10¹⁰) | week 13 |
| 5B | 1F/1M | OD | low (3 × 10¹⁰) | week 25 |
| 5C | 1F/1M | OD | medium (1 × 10¹¹) | week 13 |
| 5D | 1F/1M | OD | medium (1 × 10¹¹) | week 25 |
| 5E | 1F/1M | OD | high (2.35 × 10¹¹) | week 13 |
| 5F | 1F/1M | OD | high (2.35 × 10¹¹) | week 25 |

RNA was isolated from NHP tissues using a Direct-zol-96 RNA kit (Zymo Research). The isolated RNA was then quantified via UV/VIS spectroscopy using a Qiagen QIAxpert system. If possible, the samples were normalized to 50 ng/μL with nuclease-free water, and 4 μL containing 200 ng of the RNA were analyzed via the RT-qPCR assay. If RNA isolated from a sample could not be normalized to 50 ng/μL, then 4 μL of the neat RNA was analyzed via RT-qPCR. A RT-qPCR master mix was prepared containing specially formulated TaqMan Fast Virus 1 step Master Mix, ChRown gene specific primers and a quenched FAM-labeled probe. The RT-qPCR master mix was plated into wells of a 96 or 384 well plate. Linearized plasmid DNA dilutions were added to the wells and used as standards to be analyzed by RT-qPCR assay. When performing qPCR assay in the 384 well plate, MLP Hamilton was used to dispense Master Mix, Standards, QCs, NFW and samples while manual dispensing of each reagent were used for assays performed in a 96 well plate. The plate was then sealed and loaded onto the QuantStudio 7 Flex Real-Time PCR system and thermal cycling and simultaneous fluorescence measurement was performed. The fluorescence emitted during the RT-qPCR procedure was proportional to the amount of ChRown gene present. Once the run was complete, the fluorescence data from each well with sample was analyzed using the standard curve, and the quantity of the ChRown gene present in the sample was determined.

In addition to assessing transgene expression, the presence of ChRown DNA was also examined in the NHP tissue samples. DNA was determined using a validated NHP tissue DNA extraction procedure and quantitative polymerase chain reaction (qPCR) assay. Genomic DNA (gDNA) was isolated from NHP tissues using a KingFisher MagMAX DNA Multi-Sample Ultra 2.0 Kit with Extraction Buffer. The isolated gDNA were quantified via UV/VIS absorption method using a Qiagen QIAxpert system. If possible, the samples were normalized to 50 ng/μL with nuclease-free water, and 4 μL containing 200 ng of the gDNA was analyzed via the qPCR assay. If gDNA isolated from a sample could not be normalized to 50 ng/μL, then 4 μL of the neat gDNA was analyzed via qPCR. A qPCR master mix was prepared containing specially formulated Taqman Fast Advanced Master Mix, COCHR-3M gene specific primers and a quenched FAM-labeled probe. The qPCR master mix was plated into wells of a 96 or 384 well plate. Linearized plasmid DNA dilutions were added to the wells and used as standards to be analyzed by qPCR assay. When performing qPCR assay in the 384 well plate, MLP Hamilton was used to dispense Master Mix, Standards, QCs, NFW and samples while manual dispensing of each reagent was used for assays performed in a 96 well plate. The plate was then sealed and loaded into the QuantStudio 7 Flex Real-Time PCR system and thermal cycling and simultaneous fluorescence measurement was performed. The fluorescence emitted during the qPCR procedure was proportional to the amount of ChRown gene present in the tissue samples. Once the run was complete, the fluorescence data from each well with sample was analyzed using the standard curve and the quantity of the ChRown gene present in the sample was determined.

### Results

Administration by a single IVT injection was generally well-tolerated. There was no mortality through three months, and no systemic abnormalities were detected.

Humoral immunity as measured by serum anti-drug antibody (ADA) positive responses was limited to a total 6 of 34 animals (including one control animal) and was not dose correlated. Similarly, cellular immune responses as measured by IFN-y ELISpot analyses of peripheral blood mononuclear cells (PBMC) showed 8 of 34 animals with modestly positive responses at the 3-month timepoint. One mid-dose animal was positive at baseline.

In untreated eyes, all samples were negative for genome DNA biodistribution. Non-ocular biodistribution was sporadic and extremely limited, suggesting that very little vector escapes the eye or CNS.

In contrast, biodistribution of vector DNA was detected in all ocular tissues of vector dosed eyes, including the retina, at all dose levels. The highest levels were observed in the Iris/ciliary body, retina, RPE/Choroid and Sclera of the treated (right) eye. The same tissues in the untreated left eyes were generally below the limit of detection for vector DNA, suggesting that the vector does not migrate to the left eye after dosing in the right. Only a single sample of left RPE/Choroid showed about 4000 copies per μg DNA, however this was an isolated finding, as nearly all other tissues from left eyes were below the limit of detection.

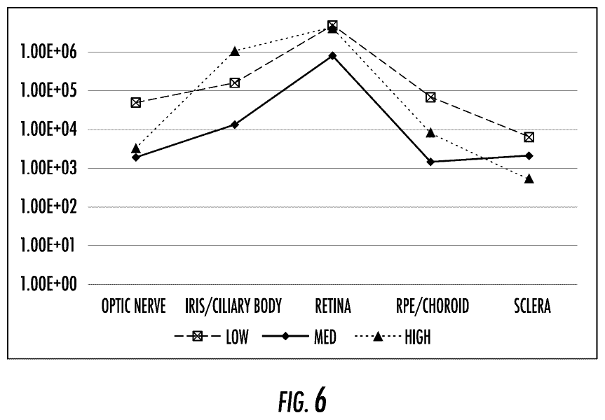

Correlated with biodistribution, high levels of gene expression were seen in all ocular tissues of the treated (right) eye at all dose levels, as shown in FIG. 6, with the highest expression seen in retina samples. A few CNS tissues including Optic Chiasm and left optic tract showed some transgene expression, mainly in the high dose group. No other notable gene expression was observed in any other systemic tissues. Systemic transgene expression was not observed. Non-ocular transgene expression was limited the contiguous visual pathways of treated eyes (optic chiasm, optic tract and lateral geniculate nucleus). Little to no expression was observed in left eye samples, except for a single sample of RPE/Choroid in a high dose female at this 13-week timepoint (1.86×10⁴ copies/μg RNA).

### Conclusions

Overall, these results indicate that vector DNA remains almost solely limited to the eye, and no transference to the untreated (left) eye occurs over the 13-week timeframe. Importantly, vector distribution is associated with high levels of transgene expression in the retina and some surrounding intraocular tissues of the treated eye out to 13 weeks post-dose.

### EXAMPLE 7

### Relationship of Dosing to Functional Efficacy

A study was conducted in the TKO mouse model to assess the relationship between viral dose and functional efficacy of ChRown.

AAV2 vectors with a 7m8 (Y444F) variant capsid driven by the CAG promoter were used to express the ChRown-GFP transgene in retinal ganglion cells. Viral vectors were injected intravitreally at doses ranging from 1.5×10⁷ vg/eye to 1.5×10¹⁰ vg/eye.

Visual functions were assessed by optomotor behavioral assays. Light sensitivity was determined as described in Example 2. Visual acuity was measured using the OptoDrum (StriaTech GmbH, Tübingen, Germany), an automatic virtual system (Benkner et al., 2013). The system is composed of four enclosed LCD monitors with an elevated animal platform in the center. The monitors display a grating stimulation that forms a virtual grating cylinder centered on the head of the animal. The light intensity of OptoDrum is fixed and is ˜30 μW/cm² at the center of the platform. The grating stimulation was presented at a contrast of 100% with rotation speed of 12 degrees/second. Presentation of the grating stimulation, change of spatial frequency, and detection of the animal's head tracking were all executed algorithmically by the system. Visual acuity was defined as the highest grating frequency that elicited OMR.

FIG. 7 illustrates the relationship between viral dose (vg/eye) and light sensitivity. The threshold light intensity decreased rapidly by about 50-fold, from 1.5×10¹⁵ (±3.2×10¹⁴; mean±SD; n=4) to 3.2×10¹³ (±6.8×10¹²; n=4) photons/cm²s, as the viral dose increased from 1.5×10⁷ to 1.5×10⁸ vg/eye. Then, as viral dose increased from 1.5×10⁸ to 7.5×10⁸ vg/eye, the threshold light intensity decreased about two-fold to 1.7×10¹³ (±2.0×10¹²; n=4) photons/cm²s. With further dose increase, the light sensitivity approached a plateau; at the highest dose tested, 1.5×10¹⁰ vg/eye, the threshold light intensity was only slightly lower (1.4×10¹³±5.0×10¹²; n=4). Threshold light intensities for doses ≥7.5×10⁸ vg/eye were not statistically different (p>0.05; one-way ANOVA).

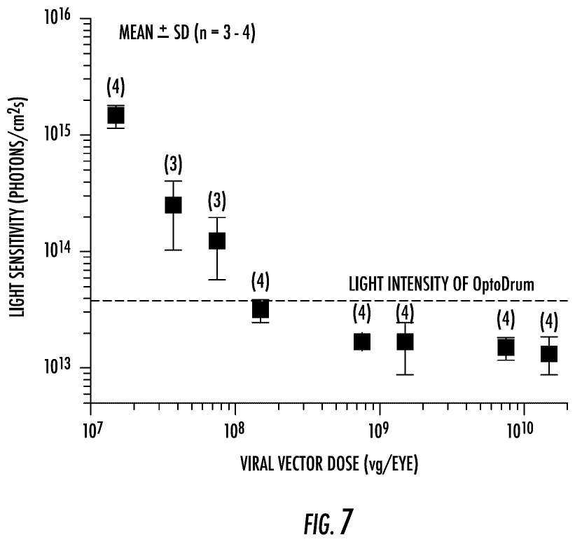

FIG. 8 depicts the relationship between the viral dose and visual acuity. OMR failed to be observed in mice injected with viral doses lower than 1.5×10⁸ vg/eye. In contrast, OMR was observed in all mice injected with viral doses at 1.5×10⁸ vg/eye and higher. The visual acuity was 0.097±0.038 cycle/degree (mean±SD; n=4) at the dose of 1.5×10⁸ vg/eye and reached 0.12-0.13 cycle/degree at higher doses. However, the visual acuity among mice receiving those higher doses was not statistically different (p>0.05; one-way ANOVA). As a control, the visual acuity for normal sighted mice (C57BL/6J) was 0.39±0.018 cycle/degree (mean±SD; n=5).

This data reveals an interesting pattern between dose and functional efficacy. The light sensitivity rapidly increased, achieving ˜50-fold difference, from the dose of 1.5×10⁷ to 1.5×10⁸ vg/eye, then approached a plateau at the dose of 7.5×10⁸ vg/eye, with values not statistically different for higher doses. This pattern is largely consistent with the visual acuity assessment. At a viral dose of <1.5×10⁸ vg/eye, OMR was not observed, so that visual acuity could not be measured. As a note here, the failure to elicit OMR in TKO mice treated with these low doses is simply due to the fact that the light intensity required to elicit OMR exceeds the intensity of the OptoDrum system which is ˜30 μW/cm² as indicated by the dashed line in FIG. 8. Meanwhile, visual acuity was measurable in TKO mice treated with viral doses ≥1.5×10⁸, but the values were not statistically different. Together, these results indicate that, for visual acuity, a saturated viral dose occurred between 1.5×10⁸-7.5×10⁸ vg/eye.

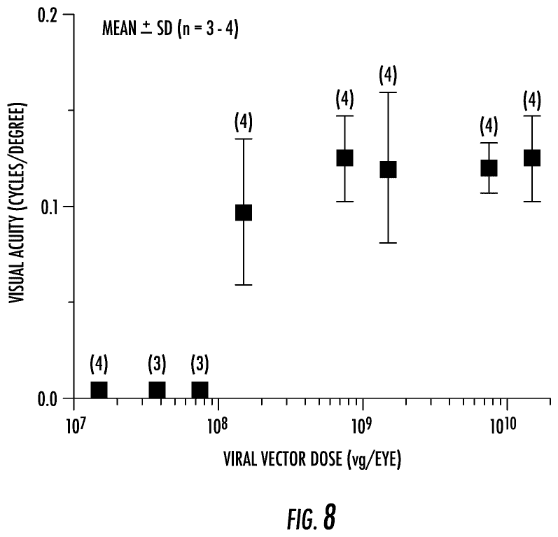

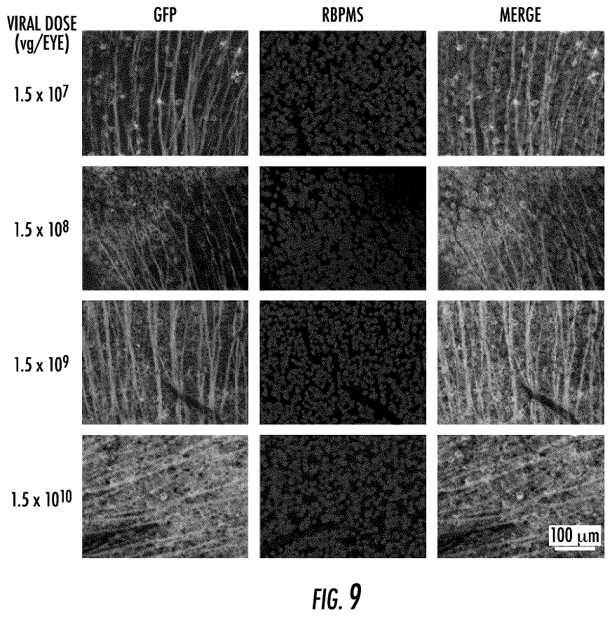

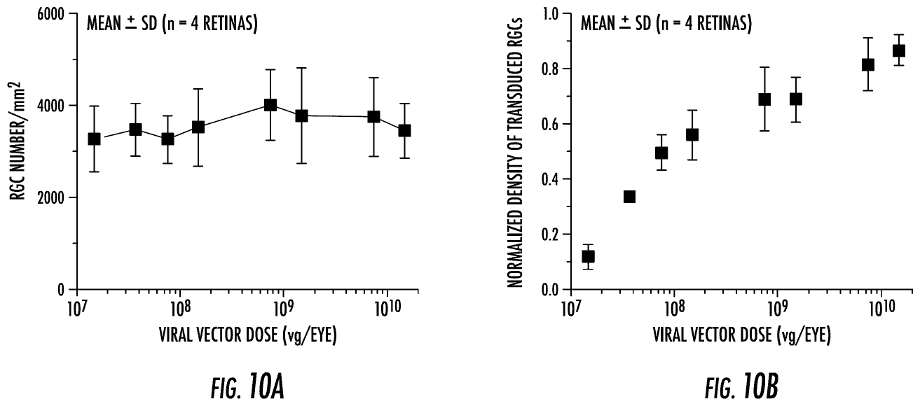

Expression of ChRown-GFP was predominantly observed in RGCs. Viral transduction efficiency in RGCs was examined by immunostaining of retinal whole mounts. FIG. 9 shows representative images of retinas injected with four different viral doses, from 1.5×10⁷ to 1.5×10¹⁰ vg/eye. Virally transduced cells were labeled with an antibody against GFP (green) while RGCs were labeled with an antibody against RBPMS (red), which is a specific marker for RGCs. Transduced RGCs were identified by the co-labeling of GFP and RBPMS. With regard to the results, first, RGC density based on RBPMS labeling was not significantly different among mice treated with different viral doses (FIG. 10A). On the other hand, viral transduction efficiency, shown in FIG. 10B as the ratio of transduced RGCs to total RGCs, increased following the increase of viral dose. Specifically, about 12%, 56%, 69%, and 87% of RGCs were respectively transduced at the viral doses of 1.5×10⁷, 1.5×10⁸, 7.5×10⁸, and 1.5×10¹⁰ vg/eye.

Viral dose-dependent protein expression of ChRown-GFP in the whole retina by Western blot assay using an antibody against GFP. For this assay, four retinas in each viral dose group were homogenized. A representative Western blot image is shown in FIG. 11A, while FIG. 11B depicts the relationship between viral dose and the relative ECL fluorescence intensity of ChRown-GFP after normalization to the ECL fluorescence intensity of β-actin (mean±SD; from three experiments). In short, the protein expression of ChRown-GFP increased with viral dose at relatively low doses, peaked at the dose of ˜1.5×10⁹ vg/eye, then decreased with further increase of viral dose.

These data suggest a capped or optimal viral dose may exist for achieving maximal functional efficacy in the TKO mouse model based on RGC expression. The information would be valuable for dose design in clinical studies. Translating it to human studies, however, requires consideration of the differences between mice and humans. To estimate the upper bounds on dose-scaling, there is an up to ˜1000× difference in vitreous volume between mice and humans (5 μL vs 4.5-5.0 mL). Accordingly, to achieve the same viral titer in the vitreous humor of humans, the corresponding optimal viral dose would be around 10¹¹ vg/eye at least. As a note, this value is close to the high dose (5×10¹¹ vg/eye) reported in one of the ongoing clinical trials. However, literature reports for mouse-to-human dose scaling suggest lower values, approaching a factor of 38-fold needed to scale from mouse to human (Schmitt et al., Mol. Pharm., 2019 Oct. 7; 16(10):4399-4404). In addition, AAV transduction efficiency in mice is largely homogeneous through the retina, while that in humans is heterogeneous, as has been reported in non-human primates (NHPs). Specifically, in NHPs, high transduction occurs in the parafoveal and far peripheral retinal regions. Furthermore, unlike in humans, mouse retinas lack fovea. It remains to be determined how these differences affect the dose and efficacy relationship.

### EXAMPLE 8

The OptoDrum system was used to measure visual acuity in TKO mice treated with the following test articles (treatment group in parenthesis):
AAV2.7m8-mGluR6-GFP-mWPRE-hGHpA (R20, negative control)
AAV2.7m8-mGluR6-ChRown-mWPRE-hGHpA (R23)
AAV2.7m8-CAG-ChRown-mWPRE-hGHpA (R03)

Mice were between 2-5 months at the time of injection. Mice receiving the R23 test article were injected with 1.5×10⁸ vg, 1.5×10⁹ vg or 1.5×10¹⁰ vg into both eyes. Mice receiving R20 or R03 were injected with 1.5×10¹⁰ vg into both eyes. Injections were performed as described in Example 4.

Mice were assessed using the OptoDrum system described in Example 4 two months and 6 months after injections.

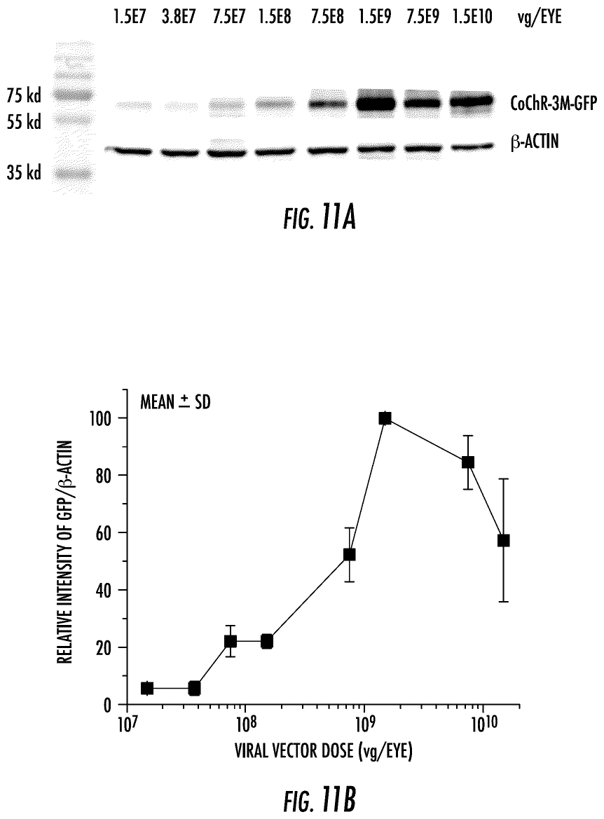

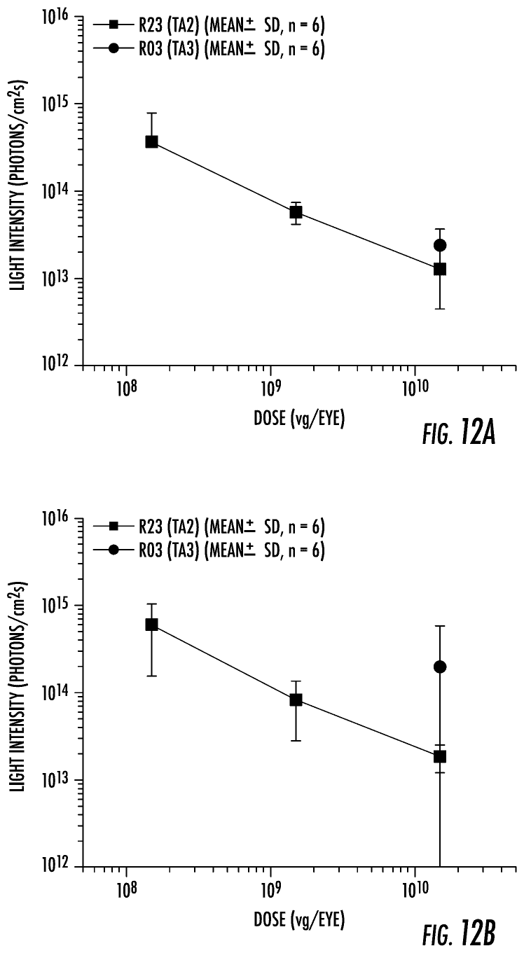

Light sensitivity: Thresholds were measured at the grating frequency at 0.042 cycle/degree. FIG. 12A shows the results of assessing threshold light intensity two months after injection. Threshold light intensity decreased with increasing doses of R23, reaching a comparable sensitivity as R03 when an equivalent dose was administered. No responses were elicited in animals that received R20 (negative control). Similar results were observed six months after injection (FIG. 12B).

Visual acuity: Visual acuity was examined using OptoMotry (CerebralMechanics Inc.), a computer-based virtual optomotor system. FIG. 13A show a dose dependent increase in visual acuity for R23 two months after injection, which persists six months after injection. After both two and six months, mice receiving the 1.5×10¹⁰ vg dose showed comparable visual acuity as mice receiving the same dose of R03 (FIG. 13A and FIG. 13B).

### EXAMPLE 9

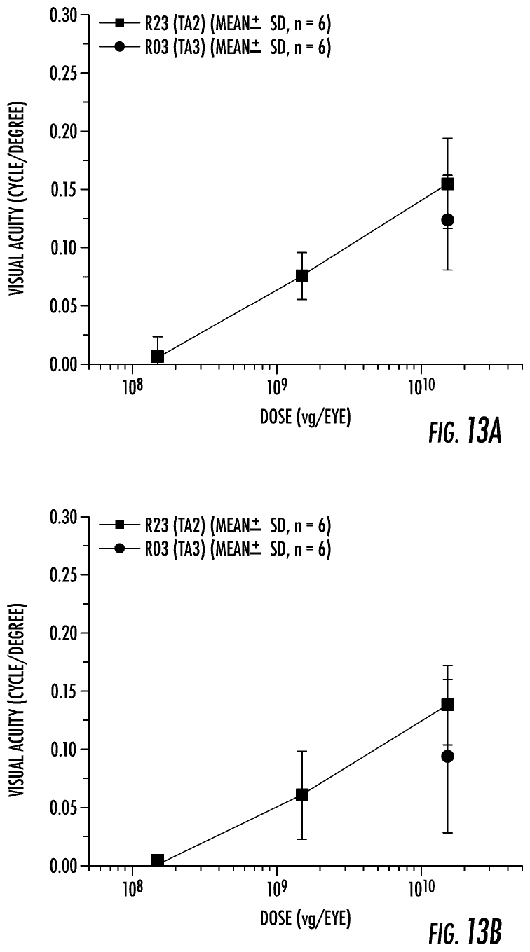

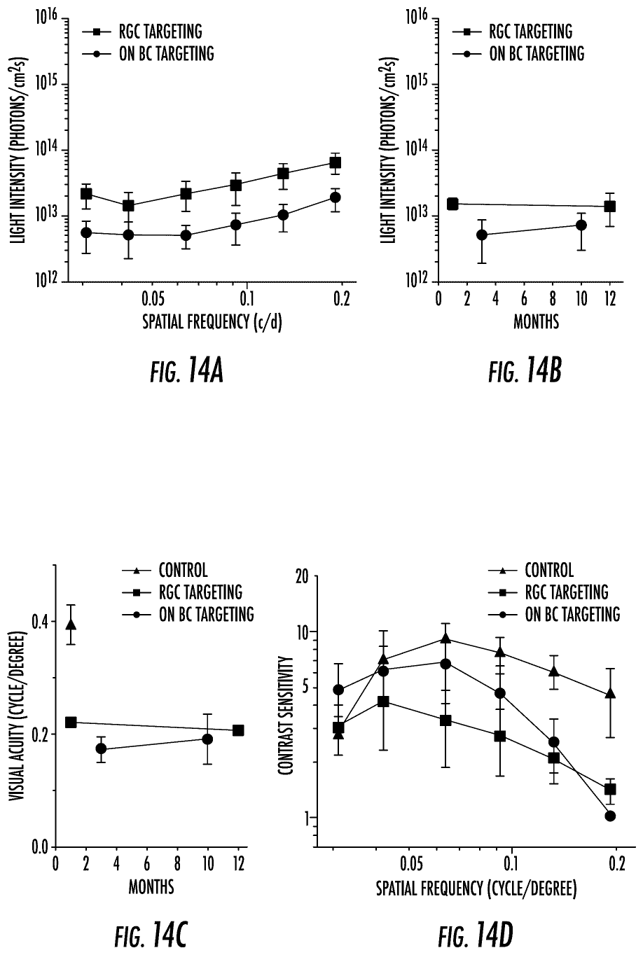

Light sensitivity and visual acuity were assessed in TKO mice treated with mGluR6-ChRown-mWPRE-hGHpA (SEQ ID NO: 31) to target ON bipolar cells. The vector was an AAV2.7m8 vector which further included a Y444F substitution. In treated TKO mice, the light intensity required to elicit OMR near the optimal spatial frequencies (0.042-0.064 cycle/degree in mice) was ˜5×10¹² photons/cm²s (FIG. 14A), which is below that of streetlighting at night. The treated TKO mice achieved an average visual acuity of 0.19 c/d, in comparison to value of 0.40 c/d in normally sighted mice (FIG. 14C). Both the light sensitivity and visual acuity were stable for up to 10 months after viral injection (FIG. 14B, FIG. 14C). Contrast sensitivity, measured using the optomotor system described in Example 2, was remarkably good, ˜15% at the peak sensitive spatial frequency (FIG. 14D). The results show the combination of the mGluR6 promoter (SEQ ID NO: 28) and ChRown is a superior viral vector for optogenetic vision restoration with ON bipolar cell targeting.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

While the invention herein disclosed has been described by means of specific embodiments and applications thereof, numerous modifications and variations could be made thereto by those skilled in the art without departing from the scope set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” means that the value may vary by +/−10%.

The term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “nucleic acid” refers to a polymer of nucleotides, e.g., deoxyribonucleotides or ribonucleotides, and may be used herein as shorthand for deoxyribonucleic acid (DNA) or ribonucleic acid (RNA).

The terms “oligonucleotide,” “nucleic acid sequence,” and “polynucleotide” are used interchangeably and are intended to include a polymeric form of nucleotides covalently linked together that may have various lengths, either deoxyribonucleotides or ribonucleotides, or analogs, derivatives or modifications thereof. An oligonucleotide is typically composed of a sequence of nucleotides comprising nucleobases selected from adenine (A), cytosine (C), guanine (G), thymine (T), and uracil (U). Thus, the term “polynucleotide sequence” may refer to the alphabetical representation of a polynucleotide molecule; alternatively, the term may be applied to the polynucleotide molecule itself.

The term “% identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. Percent identity may be determined using a computer algorithms such as the Basic Local Alignment Search Tool (“BLAST”) or a related tool, including other BLAST-based tools available at the US National Library of Medicine, National Center for Biotechnology Information website. The BLAST and related algorithms have been described, for example, in Altschul et al., 1990, J. Mol. Biol. 215:3, 403-410; and Altschul et al., 1997, Nucleic Acids Res. 25:17, 3389-402.

### INFORMAL SEQUENCE LISTING

SEQ ID NO: 1 Chloromonas oogama channelrhodopsin protein
M L G N G S A I V P I D Q C F C L A W T D S L G S D T E Q L V A N I L Q W F A
F G F S I L I L M F Y A Y Q T W R A T C G W E E V Y V C C V E L T K V I I E F F H E F
D D P S M L Y L A N G H R V Q W L R Y A E W L L T C P V I L I H L S N L T G L K D D
Y S K R T M R L L V S D V G T I V W G A T S A M S T G Y V K V I F F V L G C I Y G A
N T F F H A A K V Y I E S Y H V V P K G R P R T V V R I M A W L F F L S W G M F P V
L F V V G P E G F D A I S V Y G S T I G H T I I D L M S K N C W G L L G H Y L R V L I
H Q H I I I Y G D I R K K T K I N V A G E E M E V E T M V D Q E D E E T V
SEQ ID NO: 2 Chloromonas oogama channelrhodopsin cDNA
ATGCTGGGAAACGGCAGCGCCATTGTGCCTATCGACCAGTGCTTTTGCCTGGC
TTGGACCGACAGCCTGGGAAGCGATACAGAGCAGCTGGTGGCCAACATCCTCCAGT
GGTTCGCCTTCGGCTTCAGCATCCTGATCCTGATGTTCTACGCCTACCAGACTTGGAG
AGCCACTTGCGGTTGGGAGGAGGTCTACGTCTGTTGCGTCGAGCTGACCAAGGTCAT
CATCGAGTTCTTCCACGAGTTCGACGACCCCAGCATGCTGTACCTGGCTAACGGACA
CCGAGTCCAGTGGCTGAGATACGCAGAGTGGCTGCTGACTTGTCCCGTCATCCTGAT
CCACCTGAGCAACCTGACAGGCCTGAAGGACGACTACAGCAAGCGGACCATGAGGC
TGCTGGTGTCAGACGTGGGAACCATCGTGTGGGGAGCTACAAGCGCCATGAGCACA
GGCTACGTCAAGGTCATCTTCTTCGTGCTGGGTTGCATCTACGGCGCCAACACCTTCT
TCCACGCCGCCAAGGTGTATATCGAGAGCTACCACGTGGTGCCAAAGGGCAGACCT
AGAACCGTCGTGCGGATCATGGCTTGGCTGTTCTTCCTGTCTTGGGGCATGTTCCCCG
TGCTGTTCGTCGTGGGACCAGAAGGATTCGACGCCATCAGCGTGTACGGCTCTACCA
TTGGCCACACCATCATCGACCTCATGAGCAAGAATTGTTGGGGCCTGCTGGGACACT
ATCTGAGAGTGCTGATCCACCAGCACATCATCATCTACGGCGACATCCGCAAGAAGA
CCAAGATCAACGTGGCCGGCGAGGAGATGGAAGTGGAGACCATGGTGGACCAGGAG
GACGAGGAGACAGTG
SEQ ID NO: 3 CoChR L112C
M L G N G S A I V P I D Q C F C L A W T D S L G S D T E Q L V A N I L Q W F A
F G F S I L I L M F Y A Y Q T W R A T C G W E E V Y V C C V E L T K V I I E F F H E F
D D P S M L Y L A N G H R V Q W L R Y A E W L L T C P V I C I H L S N L T G L K D D
Y S K R T M R L L V S D V G T I V W G A T S A M S T G Y V K V I F F V L G C I Y G A
N T F F H A A K V Y I E S Y H V V P K G R P R T V V R I M A W L F F L S W G M F P V
L F V V G P E G F D A I S V Y G S T I G H T I I D L M S K N C W G L L G H Y L R V L I
H Q H I I I Y G D I R K K T K I N V A G E E M E V E T M V D Q E D E E T V
SEQ ID NO: 4 CoChR L112C/T139C
M L G N G S A I V P I D Q C F C L A W T D S L G S D T E Q L V A N I L Q W F A
F G F S I L I L M F Y A Y Q T W R A T C G W E E V Y V C C V E L T K V I I E F F H E F
D D P S M L Y L A N G H R V Q W L R Y A E W L L T C P V I C I H L S N L T G L K D D
Y S K R T M R L L V S D V G C I V W G A T S A M S T G Y V K V I F F V L G C I Y G A
N T F F H A A K V Y I E S Y H V V P K G R P R T V V R I M A W L F F L S W G M F P V
L F V V G P E G F D A I S V Y G S T I G H T I I D L M S K N C W G L L G H Y L R V L I
H Q H I I I Y G D I R K K T K I N V A G E E M E V E T M V D Q E D E E T V
SEQ ID NO: 5 Chrown
M L G N G S A I V P I D Q C F C L A W T D S L G S D T E Q L V A N I L Q W F A
F G F S I L I L M F Y A Y Q T W R A T C G W E E V Y V C C V E L T K V I I E F F H E F
D D P S M L Y L A N G E R V Q W L R Y A E W L L T C P V I C I H L S N L T G L K D D
Y S K R T M R L L V S D V G T I V W G A T S A M S T G Y V K V I F F V L G C I Y G A
N T F F H A A K V Y I E S Y H V V P K G R P R T V V R I M A W L F F L S W G M F P V
L F V V G P E G F D A I S V Y G S T I G H T I I D L M S K N C W G L L G H Y L R V L I
H Q H I I I Y G D I R K T T K I N V A G E E M E V E T M V D Q E D E E T V
SEQ ID NO: 6 AAV2 VP1 capsid protein
M A A D G Y L P D W L E D T L S E G I R Q W W K L K P G P P P P K P A E R H
K D D S R G L V L P G Y K Y L G P F N G L D K G E P V N E A D A A A L E H D K A Y
D R Q L D S G D N P Y L K Y N H A D A E F E R L K E D T S F G G N L G R A V F Q A K
K R V L E P L G L V E E P V K T A P G K K R P V E H S P V E P D S S S G T G K A G Q
Q P A R K R L N F G Q T G D A D S V P D P Q P L G Q P P A A P S G L G N T M A T G S
G A P M A D N N E G A D G V G N S S G N W H C D S T W M G D R V I T T S T R T W A
L P T Y N N H L Y K Q I S S Q S G A S N D N H Y F G Y S T P W G Y F D F N R F H C H
F S P R D W Q L I N N N W G F R P K R L N F K L F N I Q V K E V T Q N D G T T T I A
N N L T S T V Q V F T D S E Y Q L P Y V L G S A H Q G C L P P F P A D V F M V P Q Y
G Y L T L N N G S Q A V G R S S F Y C E Y F P S Q M L R T G N N F T F S Y T F E D V P
F H S S Y A H S Q S L D R L M N P L I D Q Y L Y Y L S R T N T P S G T T T Q S R L Q F
S Q A G A S D I R D Q S R N W L P G P C Y R Q Q R V S K T S A N N N S E Y S W T G A
T K Y H L N G R D S L V N P G P A M A S H K D D E E K F F P Q S G V L I F G K Q G S
E K T N V D I E K V M I T D E E E I R T T N P V A T E Q Y G S V S T N L Q R N R Q A A
T A D V N T Q G V L P G M V W Q D R D V Y L Q G P I W A K I P H T D G H F H P S P L
M G G F G L K H P P P Q I L I K N T P V P A N P S T T F S A A K F A S F I T Q Y S T G Q
V S V E I E W E Q K E N S K R W N P E I Q Y T S N Y N K S V N V D F T V D I N G V Y
S E P R P I G T R Y L T R N L
SEQ ID NO: 7 7M8 (fragment of AAV2 VP1)
M A T G S G A P M A D N N E G A D G V G N S S G N W H C D S T W M G D R V
I T T S T R T W A L P T Y N N H L Y K Q I S S Q S G A S N D N H Y F G Y S T P W G Y
F D F N R F H C H F S P R D W Q R L I N N N W G F R P K R L N F K L F N I Q V K E V
T Q N D G T T T I A N N L T S T V Q V F T D S E Y Q L P Y V L G S A H Q G C L P P F P
A D V F M V P Q Y G Y L T L N N G S Q A V G R S S F Y C L E Y F P S Q M L R T G N N
F T F S Y T F E D V P F H S S Y A H S Q S L D R L M N P L I D Q Y L Y Y L S R T N T P
S G T T T Q S R L Q F S Q A G A S D I R D Q S R N W L P G P C Y R Q Q R V S K T S A
D N N N S E Y S W T G A T K Y H L N G R D S L V N P G P A M A S H K D D E E K F F
P Q S G V L I F G K Q G S E K T N V D I E K V M I T D E E E I R T T N P V A T E Q Y G
S V S T N L Q R G N L A L G E T T R P A R Q A A T A D V N T Q G V L P G M V W Q D
R D V Y L Q G P I W A K I P H T D G H F H P S P L M G G F G L K H P P P Q I L I K N T
P V P A N P S T T F S A A K F A S F I T Q Y S T G Q V S V E I E W E L Q K E N S K R W
N P E I Q Y T S N Y N K S V N V D F T V D I N G V Y S E P R P I G T R Y L T R N L
SEQ ID NO: 8 Dalkara 7m8 peptide
L G E T T R P
SEQ ID NO: 9 4DMT peptide id49 from WO2021222148
L A I S D Q T K H A
SEQ ID NO: 10 CAG promoter
ctagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaa
SEQ ID NO: 11_Human mGluR6 1784 bp promoter fragment
caagcaggaggctgctgtgtgctgggagctgtcaggctcgtcctgaacagggaagggcccatccacctcccaaacccagttt
atgcagtccttcgcaatgtcaggctcagggcctggcaccagccaagctccccacccttcccactgttaaaatggataggagcagggctag
gcccagcctgttgactctgggcttccaccaggagaagtggttctggcagtagaaactatcggggcctgggagaggcgggggaagaga
gaaaggtggcatgtttcttgcttgctccctctaccagccttgtccaaatccccgcagccaccctaatccagcctgtctaatggagcccaagc
cggctcaggccctcggacgaggagcctgctaatccctgtggctaggagctcaccacctgtctccaggacgccctttgctctcttggcatca
gagagccaaatcctgggcctcggatggggggatgataaaagcatcttttggccaagccccctcaccttggcctccacgatgagatggg
agttaggtgcagagagcgttggcacagtgagcaccgcagctcgagtggctgcctcagacccagagcccgaggagactttatacggagc
cagaacgaccccgcggggttccatcctcccaagcaataggcgggagtgggagctgcgaggaaagccggcccctcccctccctccatc
caaggcagtgtgggctgtttgtttcatgccattctgggtgtgaatcctgatgcccacacatgccagctgcatgcacttgggcaactcaactc
actcctcgagggctgtttctcgactgcagggtgttgtaagttcgctaatactaaaggcttctccctcctggccccttcctgcccctcgctcttc
ctcctcttccttaggccctcccagctcaggcagcccctgccccctgcagggttctgcaaggagaaagctggggaataccttaggcaactg
cagtcaggagcactggtggccaggacagagacagagagacagaaaaggggtcagggacagagagagataaccgcagggagagac
aggaagggacagagacagaaaagatttccaagaagaggacagaggcagaaagccagggacagagactgagaaacagagacctaga
ggcagaagaagactgagatagagatggacagagattgtgtcagacacagccccagagacagccagacagtctgagtcagacgcaaac
caaagacaagaaaacaggaaaacagacccagagattgggagagggaggggaaggagatgcggggagagccagcaccgccacccc
ccacactcaggaggggtctccaccctcggagcggtctctcatccctccctagaatccttaaatcctctctcgctcagggcctcggccgcat
ctgtcacagacttgtcctgaaccgacagcggctggcgcaggtgactggcttggggcgggagcctgggtgtgcgctggggatggacccc
gaggaagaggggccaagctgtcgggaagcggcagggctggaggggtggaggcagtggtcgggcgggaccccgggcgacagggtt
cggcgcttgtaagagcgagacggaggcccgggcaggccggctgagctaactccccagagccgaagtggaaggcgcgccccgagcg
ccttctccccaggaccccggtgtccctccccgcgccccgagcccgcgctctccttcccccgccctcagagcgctccccgcccctctgtct
ccccgcagcccgctagacgagccg
SEQ ID NO: 12 Human mGluR6 547 bp promoter fragment
ccaaagacaagaaaacaggaaaacagacccagagattgggagagggaggggaaggagatgcggggagagccagcacc
gccaccccccacactcaggaggggtctccaccctcggagcggtctctcatccctccctagaatccttaaatcctctctcgctcagggcctc
ggccgcatctgtcacagacttgtcctgaaccgacagcggctggcgcaggtgactggcttggggcgggagcctgggtgtgcgctgggga
tggaccccgaggaagaggggccaagctgtcgggaagcggcagggctggaggggtggaggcagtggtcgggcgggaccccgggcg
acagggttcggcgcttgtaagagcgagacggaggcccgggcaggccggctgagctaactccccagagccgaagtggaaggcgcgc
cccgagcgccttctccccaggaccccggtgtccctccccgcgccccgagcccgcgctctccttcccccgccctcagagcgctccccgc
ccctctgtctccccgcagcccgctagacgagccg
SEQ ID NO: 13 Human mGluR6 198 bp enhancer
gatccttagattatgaaacatttacaattatgaatgaatattagatgttatcaaatgctttttctgcatccatttagataatcatgtttttcc
tttaatctgttaatgcggtgaattacattaatagatttcctaagtcattaatctgctaaagtgcatttctgggacaaaccagacttggttatgacat
tgtatgta
SEQ ID NO: 14 WPRE
gataatcaacctctggattacaaaatttgtgaaagattgactggtattcttaactatgttgctccttttacgctatgtggatacgctgct
ttaatgcctttgtatcatgctattgcttcccgtatggctttcattttctcctccttgtataaatcctggttgctgtctctttatgaggagttgtggccc
gttgtcaggcaacgtggcgtggtgtgcactgtgtttgctgacgcaacccccactggttggggcattgccaccacctgtcagctcctttccgg
gactttcgctttccccctccctattgccacggcggaactcatcgccgcctgccttgcccgctgctggacaggggctcggctgttgggcact
gacaattccgtggtgttgtcggggaaatcatcgtcctttccttggctgctcgcctgtgttgccacctggattctgcgcgggacgtccttctgct
acgtcccttcggccctcaatccagcggaccttccttcccgcggcctgctgccggctctgcggcctcttccgcgtcttcgccttcgccctcag
acgagtcggatctccctttgggccgcctccccgcctgatgcgg
SEQ ID NO: 15 5′ ITR
ctgcgcgctcgctcgctcactgaggccgcccgggcaaagcccgggcgtcgggcgacctttggtcgcccggcctcagtgagc
gagcgagcgcgcagagagggagtggccaactccatcactaggggttcct
SEQ ID NO: 16 3′ ITR
aggaacccctagtgatggagttggccactccctctctgcgcgctcgctcgctcactgaggccgggcgaccaaaggtcgcccg
acgcccgggctttgcccgggcggcctcagtgagcgagcgagcgcgcag
SEQ ID NO: 17 Chrown DNA
atgctgggcaacggatctgctatcgttcctatcgaccagtgcttctgcctggcttggaccgactctctgggctctgataccgaac
agctggtggccaatatcctgcagtggttcgccttcggcttcagcattctgatcctgatgttctacgcctaccagacctggcgggccacctgt
ggctgggaggaagtgtatgtgtgctgcgtggaactgacaaaggtgatcatcgagttcttccacgagttcgacgaccccagcatgctgtac
ctggccaatggcgagagagtgcagtggctgcggtacgccgagtggctgctgacctgtcctgtcatctgcatccacctgtccaacctgacc
ggcctgaaggacgattacagcaaaagaaccatgagactgctcgtgtccgatgtgggcaccatcgtgtggggcgctaccagcgctatgag
caccggctacgtgaaggttatcttctttgtgctgggttgcatctatggcgccaacactttcttccacgccgccaaggtgtacatcgagagcta
ccacgtggtccccaagggcagacctagaacagtggtgcggatcatggcctggctgtttttcctgagctggggaatgtttccagtgctgttc
gtggtgggccctgagggatttgatgccatcagcgtgtacggcagcacaatcggccatacaatcattgacctgatgagcaagaactgttgg
ggcctgctgggacactacttgagggtgctgatccaccagcacatcatcatctacggcgacatcagaaagaccaccaaaatcaacgtggc
cggagaagaaatggaagtcgagacaatggtggaccaagaggacgaggaaacagtg
SEQ ID NO: 18 hGHpA
gggtggcatccctgtgacccctccccagtgcctctcctggccctggaagttgccactccagtgcccaccagccttgtcctaata
aaattaagttgcatcattttgtctgactaggtgtccttctataatattatggggtggaggggggtggtatggagcaaggggcaagttgggaa
gacaacctgtagggcctgcggggtctattgggaaccaagctggagtgcagtggcacaatcttggctcactgcaatctccgcctcctgggt
tcaagcgattctcctgcctcagcctcccgagttgttgggattccaggcatgcatgaccaggctcagctaatttttgtttttttggtagagacgg
ggtttcaccatattggccaggctggtctccaactcctaatctcaggtgatctacccaccttggcctcccaaattgctgggattacaggcgtga
accactgctcccttccctgtcctt
SEQ ID NO: 19 pCAG-Chrown-GFP-mWPRE-hGHpA
gacgcgccctgtagcggcgcattaagcgcggcgggtgtggtggttacgcgcagcgtgaccgctacacttgccagcgcccta
gcgcccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttcgccc
tttgacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgtttacaatttcaggtg
gcacttttcggggaaatgtgcgcggaacccctatttgtttatttttctaaatacattcaaatatgtatccgctcatgagacaataaccctgataaa
tgcttcaataatattgaaaaaggaagagtatgagtattcaacatttccgtgtcgcccttattcccttttttgcggcattttgccttcctgtttttgctc
acccagaaacgctggtgaaagtaaaagatgctgaagatcagttgggtgcacgagtgggttacatcgaactggatctcaacagcggtaag
atccttgagagttttcgccccgaagaacgttttccaatgatgagcacttttaaagttctgctatgtggcgcggtattatcccgtattgacgccg
ggcaagagcaactcggtcgccgcatacactattctcagaatgacttggttgagtactcaccagtcacagaaaagcatcttacggatggcat
gacagtaagagaattatgcagtgctgccataaccatgagtgataacactgcggccaacttacttctgacaacgatcggaggaccgaagga
gctaaccgcttttttgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttcccggcaacaattaat
agactggatggaggcggataaagttgcaggaccacttctgcgctcggcccttccggctggctggtttattgctgataaatctggagccggt
gagcgtgggtctcgcggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaagtttactcatatatacttt
agattgatttaaaacttcatttttaatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgcttgcaaacaaaaaaacca
ccgctaccagcggtggtttgtttgccggatcaagagctaccaactctttttccgaaggtaactggcttcagcagagcgcagataccaaatac
tgtccttctagtgtagccgtagttaggccaccacttcaagaactctgtagcaccgcctacatacctcgctctgctaatcctgttaccagtggct
gctgccagtggcgataagtcgtgtcttaccgggttggactcaagacgatagttaccggataaggcgcagcggtcgggctgaacggggg
gttcgtgcacacagcccagcttggagcgaacgacctacaccgaactgagatacctacagcgtgagcattgagaaagcgccacgcttccc
gaagggagaaaggcggacaggtatccggtaagcggcagggtcggaacaggagagcgcacgagggagcttccagggggaaacgcc
tggtatctttatagtcctgtcgggtttcgccacctctgacttgagcgtcgatttttgtgatgctcgtcaggggggcggagcctatggaaaaac
gccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgc
ctgatgcggtattttctccttacgcatctgtgcggtatttcacaccgcagaccagccgcgtaacctggcaaaatcggttacggttgagtaata
aatggatgccctgcgtaagcgggtgtgggcggacaataaagtcttaaactgaacaaaatagatctaaactatgacaataaagtcttaaact
agacagaatagttgtaaactgaaatcagtccagttatgctgtgaaaaagcatactggacttttgttatggctaaagcaaactcttcattttctga
agtgcaaattgcccgtcgtattaaagaggggcgtggccaagggcatggtaaagactatattcgcggcgttgtgacaatttaccgaacaact
ccgcggccgggaagccgatctcggcttgaacgaattgttaggtggcggtacttgggtcgatatcaaagtgcatcacttcttcccgtatgcc
caactttgtatagagagccactgcgggatcgtcaccgtaatctgcttgcacgtagatcacataagcaccaagcgcgttggcctcatgcttga
ggagattgatgagcgcggtggcaatgccctgcctccggtgctcgccggagactgcgagatcatagatatagatctcactacgcggctgct
caaacctgggcagaacgtaagccgcgagagcgccaacaaccgcttcttggtcgaaggcagcaagcgcgatgaatgtcttactacggag
caagttcccgaggtaatcggagtccggctgatgttgggagtaggtggctacgtctccgaactcacgaccgaaaagatcaagagcagccc
gcatggatttgacttggtcagggccgagcctacatgtgcgaatgatgcccatacttgagccacctaactttgttttagggcgactgccctgct
gcgtaacatcgttgctgctgcgtaacatcgttgctgctccataacatcaaacatcgacccacggcgtaacgcgcttgctgcttggatgcccg
aggcatagactgtacaaaaaaacagtcataacaagccatgaaaaccgccactgcgccgttaccaccgctgcgttcggtcaaggttctgga
ccagttgcgtgagcgcatacgctacttgcattacagtttacgaaccgaacaggcttatgtcaactgggttcgtgccttcatccgtttccacgg
tgtgcgtcacccggcaaccttgggcagcagcgaagtcgaggcatttctgtcctggctggcgaacgagcgcaaggtttcggtctccacgc
atcgtcaggcattggcggccttgctgttcttctacggcaaggtgctgtgcacggatctgccctggcttcaggagatcggaagacctcggcc
gtcgcggcgcttgccggtggtgctgaccccggatgaagtggttcgcatcctcggttttctggaaggcgagcatcgtttgttcgcccaggac
tctagctatagttctagtggttggctacagcttgcatgcctgcaggcagctgcgcgctcgctcgctcactgaggccgcccgggcgtcggg
cgacctttggtcgcccggcctcagtgagcgagcgagcgcgcagagagggagtggccaactccatcactaggggttcctgcggccgca
cgcgtctagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccattgacgtc
aatgggtggagtatttacggtaaactgcccacttggcagtacatcaagtgtatcatatgccaagtacgccccctattgacgtcaatgacggt
aaatggcccgcctggcattatgcccagtacatgaccttatgggactttcctacttggcagtacatctacgtattagtcatcgctattaccatgg
tcgaggtgagccccacgttctgcttcactctccccatctcccccccctccccacccccaattttgtatttatttattttttaattattttgtgcagcg
atgggggcggggggggggggggggcgcgcgccaggcggggcggggcggggcgaggggcggggcggggcgaggcggagagg
tgcggcggcagccaatcagagcggcgcgctccgaaagtttccttttatggcgaggcggcggcggcggcggccctataaaaagcgaag
cgcgcggcgggcgggagtcgctgcgcgctgccttcgccccgtgccccgctccgccgccgcctcgcgccgcccgccccggctctgact
gaccgcgttactcccacaggtgagcgggcgggacggcccttctcctccgggctgtaattagcgcttggtttaatgacggcttgtttcttttct
gtggctgcgtgaaagccttgaggggctccgggagggccctttgtgcggggggagcggctcggggggtgcgtgcgtgtgtgtgtgcgtg
gggagcgccgcgtgcggctccgcgctgcccggcggctgtgagcgctgcgggcgcggcgcggggctttgtgcgctccgcagtgtgcg
cgaggggagcgcggccgggggcggtgccccgcggtgcggggggggctgcgaggggaacaaaggctgcgtgcggggtgtgtgcgt
gggggggtgagcagggggtgtgggcgcgtcggtcgggctgcaaccccccctgcacccccctccccgagttgctgagcacggcccgg
cttcgggtgcggggctccgtacggggcgtggcgcggggctcgccgtgccgggcggggggtggcggcaggtgggggtgccgggcg
gggcggggccgcctcgggccggggagggctcgggggaggggcgcggcggcccccggagcgccggcggctgtcgaggcgcggc
gagccgcagccattgccttttatggtaatcgtgcgagagggcgcagggacttcctttgtcccaaatctgtgcggagccgaaatctgggag
gcgccgccgcaccccctctagcgggcgcggggcgaagcggtgcggcgccggcaggaaggaaatgggcggggagggccttcgtgc
gtcgccgcgccgccgtccccttctccctctccagcctcggggctgtccgcggggggacggctgccttcgggggggacggggcagggc
ggggttcggcttctggcgtgtgaccggcggctctagagcctctgctaaccatgttcatgccttcttctttttcctacagctcctgggcaacgtg
ctggttattgtgctgtctcatcattttggcaaagagctcgcgatccgccaccatgctgggcaacggatctgctatcgttcctatcgaccagtg
cttctgcctggcttggaccgactctctgggctctgataccgaacagctggtggccaatatcctgcagtggttcgccttcggcttcagcattct
gatcctgatgttctacgcctaccagacctggcgggccacctgtggctgggaggaagtgtatgtgtgctgcgtggaactgacaaaggtgat
catcgagttcttccacgagttcgacgaccccagcatgctgtacctggccaatggcgagagagtgcagtggctgcggtacgccgagtggc
tgctgacctgtcctgtcatctgcatccacctgtccaacctgaccggcctgaaggacgattacagcaaaagaaccatgagactgctcgtgtc
cgatgtgggcaccatcgtgtggggcgctaccagcgctatgagcaccggctacgtgaaggttatcttctttgtgctgggttgcatctatggcg
ccaacactttcttccacgccgccaaggtgtacatcgagagctaccacgtggtccccaagggcagacctagaacagtggtgcggatcatg
gcctggctgtttttcctgagctggggaatgtttccagtgctgttcgtggtgggccctgagggatttgatgccatcagcgtgtacggcagcac
aatcggccatacaatcattgacctgatgagcaagaactgttggggcctgctgggacactacttgagggtgctgatccaccagcacatcatc
atctacggcgacatcagaaagaccaccaaaatcaacgtggccggagaagaaatggaagtcgagacaatggtggaccaagaggacga
ggaaacagtggaattcggaggcggaggtggagctagcaaaggagaagaactcttcactggagttgtcccaattcttgttgaattagatggt
gatgttaacggccacaagttctctgtcagtggagagggtgaaggtgatgcaacatacggaaaacttaccctgaagttcatctgcactactg
gcaaactgcctgttccatggccaacactagtcactactctgtgctatggtgttcaatgcttttcaagatacccggatcatatgaaacggcatg
actttttcaagagtgccatgcccgaaggttatgtacaggaaaggaccatcttcttcaaagatgacggcaactacaagacacgtgctgaagt
caagtttgaaggtgatacccttgttaatagaatcgagttaaaaggtattgacttcaaggaagatggcaacattctgggacacaaattggaata
caactataactcacacaatgtatacatcatggcagacaaacaaaagaatggaatcaaagtgaacttcaagacccgccacaacattgaagat
ggaagcgttcaactagcagaccattatcaacaaaatactccaattggcgatggccctgtccttttaccagacaaccattacctgtccacaca
atctgccctttcgaaagatcccaacgaaaagagagaccacatggtccttcttgagtttgtaacagctgctgggattacacatggcatggatg
aactgtacaactaataactcgagggtaaccgtggtaccgataatcaacctctggattacaaaatttgtgaaagattgactggtattcttaacta
tgttgctccttttacgctatgtggatacgctgctttaatgcctttgtatcatgctattgcttcccgtatggctttcattttctcctccttgtataaatcct
ggttgctgtctctttatgaggagttgtggcccgttgtcaggcaacgtggcgtggtgtgcactgtgtttgctgacgcaacccccactggttgg
ggcattgccaccacctgtcagctcctttccgggactttcgctttccccctccctattgccacggcggaactcatcgccgcctgccttgcccg
ctgctggacaggggctcggctgttgggcactgacaattccgtggtgttgtcggggaaatcatcgtcctttccttggctgctcgcctgtgttgc
cacctggattctgcgcgggacgtccttctgctacgtcccttcggccctcaatccagcggaccttccttcccgcggcctgctgccggctctg
cggcctcttccgcgtcttcgccttcgccctcagacgagtcggatctccctttgggccgcctccccgcctgatgcggggatccggtaacgtc
gagagatctacgggtggcatccctgtgacccctccccagtgcctctcctggccctggaagttgccactccagtgcccaccagccttgtcct
aataaaattaagttgcatcattttgtctgactaggtgtccttctataatattatggggtggaggggggtggtatggagcaaggggcaagttgg
gaagacaacctgtagggcctgcggggtctattgggaaccaagctggagtgcagtggcacaatcttggctcactgcaatctccgcctcctg
ggttcaagcgattctcctgcctcagcctcccgagttgttgggattccaggcatgcatgaccaggctcagctaatttttgtttttttggtagagac
ggggtttcaccatattggccaggctggtctccaactcctaatctcaggtgatctacccaccttggcctcccaaattgctgggattacaggcgt
gaaccactgctcccttccctgtccttctgattttgtaggtaaccacgtgcggaccgagcggccgcaggaacccctagtgatggagttggcc
actccctctctgcgcgctcgctcgctcactgaggccgggcgaccaaaggtcgcccgacgcccgggctttgcccgggcggcctcagtga
gcgagcgagcgcgcagctgcctgcaggtctgagacaataaccctgataaatgcttcaataatgtaagcttgtcgagaagtactagaggat
cataatcagccataccacatttgtagaggttttacttgctttaaaaaacctcccacacctccccctgaacctgaaacataaaatgaatgcaatt
gttgttgttaacttgtttattgcagcttataatggttacaaataaagcaatagcatcacaaatttcacaaataaagcatttttttcactgcattctag
ttgtggtttgtccaaactcatcaatgtatcttatcatgtctggatctgatcactgatatcgcctaggagatccgaaccagataagtgaaatctag
ttccaaactattttgtcatttttaattttcgtattagcttacgacgctacacccagttcccatctattttgtcactcttccctaaataatccttaaaaac
tccatttccacccctcccagttcccaactattttgtccgcccacagcggggcatttttcttcctgttatgtttttaatcaaacatcctgccaactcc
atgtgacaaaccgtcatcttcggctactttttctctgtcacagaatgaaaatttttctgtcatctcttcgttattaatgtttgtaattgactgaatatc
aacgcttatttgcagcctgaatggcgaatg
SEQ ID NO: 20 pCAG-Chrown-GFP-hGHpA
gacgcgccctgtagcggcgcattaagcgcggcgggtgtggtggttacgcgcagcgtgaccgctacacttgccagcgcccta
gcgcccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttcgccc
tttgacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgtttacaatttcaggtg
gcacttttcggggaaatgtgcgcggaacccctatttgtttatttttctaaatacattcaaatatgtatccgctcatgagacaataaccctgataaa
tgcttcaataatattgaaaaaggaagagtatgagtattcaacatttccgtgtcgcccttattcccttttttgcggcattttgccttcctgtttttgctc
acccagaaacgctggtgaaagtaaaagatgctgaagatcagttgggtgcacgagtgggttacatcgaactggatctcaacagcggtaag
atccttgagagttttcgccccgaagaacgttttccaatgatgagcacttttaaagttctgctatgtggcgcggtattatcccgtattgacgccg
ggcaagagcaactcggtcgccgcatacactattctcagaatgacttggttgagtactcaccagtcacagaaaagcatcttacggatggcat
gacagtaagagaattatgcagtgctgccataaccatgagtgataacactgcggccaacttacttctgacaacgatcggaggaccgaagga
gctaaccgcttttttgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttcccggcaacaattaat
agactggatggaggcggataaagttgcaggaccacttctgcgctcggcccttccggctggctggtttattgctgataaatctggagccggt
gagcgtgggtctcgcggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaagtttactcatatatacttt
agattgatttaaaacttcatttttaatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgcttgcaaacaaaaaaacca
ccgctaccagcggtggtttgtttgccggatcaagagctaccaactctttttccgaaggtaactggcttcagcagagcgcagataccaaatac
tgtccttctagtgtagccgtagttaggccaccacttcaagaactctgtagcaccgcctacatacctcgctctgctaatcctgttaccagtggct
gctgccagtggcgataagtcgtgtcttaccgggttggactcaagacgatagttaccggataaggcgcagcggtcgggctgaacggggg
gttcgtgcacacagcccagcttggagcgaacgacctacaccgaactgagatacctacagcgtgagcattgagaaagcgccacgcttccc
gaagggagaaaggcggacaggtatccggtaagcggcagggtcggaacaggagagcgcacgagggagcttccagggggaaacgcc
tggtatctttatagtcctgtcgggtttcgccacctctgacttgagcgtcgatttttgtgatgctcgtcaggggggcggagcctatggaaaaac
gccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgc
ctgatgcggtattttctccttacgcatctgtgcggtatttcacaccgcagaccagccgcgtaacctggcaaaatcggttacggttgagtaata
aatggatgccctgcgtaagcgggtgtgggcggacaataaagtcttaaactgaacaaaatagatctaaactatgacaataaagtcttaaact
agacagaatagttgtaaactgaaatcagtccagttatgctgtgaaaaagcatactggacttttgttatggctaaagcaaactcttcattttctga
agtgcaaattgcccgtcgtattaaagaggggcgtggccaagggcatggtaaagactatattcgcggcgttgtgacaatttaccgaacaact
ccgcggccgggaagccgatctcggcttgaacgaattgttaggtggcggtacttgggtcgatatcaaagtgcatcacttcttcccgtatgcc
caactttgtatagagagccactgcgggatcgtcaccgtaatctgcttgcacgtagatcacataagcaccaagcgcgttggcctcatgcttga
ggagattgatgagcgcggtggcaatgccctgcctccggtgctcgccggagactgcgagatcatagatatagatctcactacgcggctgct
caaacctgggcagaacgtaagccgcgagagcgccaacaaccgcttcttggtcgaaggcagcaagcgcgatgaatgtcttactacggag
caagttcccgaggtaatcggagtccggctgatgttgggagtaggtggctacgtctccgaactcacgaccgaaaagatcaagagcagccc
gcatggatttgacttggtcagggccgagcctacatgtgcgaatgatgcccatacttgagccacctaactttgttttagggcgactgccctgct
gcgtaacatcgttgctgctgcgtaacatcgttgctgctccataacatcaaacatcgacccacggcgtaacgcgcttgctgcttggatgcccg
aggcatagactgtacaaaaaaacagtcataacaagccatgaaaaccgccactgcgccgttaccaccgctgcgttcggtcaaggttctgga
ccagttgcgtgagcgcatacgctacttgcattacagtttacgaaccgaacaggcttatgtcaactgggttcgtgccttcatccgtttccacgg
tgtgcgtcacccggcaaccttgggcagcagcgaagtcgaggcatttctgtcctggctggcgaacgagcgcaaggtttcggtctccacgc
atcgtcaggcattggcggccttgctgttcttctacggcaaggtgctgtgcacggatctgccctggcttcaggagatcggaagacctcggcc
gtcgcggcgcttgccggtggtgctgaccccggatgaagtggttcgcatcctcggttttctggaaggcgagcatcgtttgttcgcccaggac
tctagctatagttctagtggttggctacagcttgcatgcctgcaggcagctgcgcgctcgctcgctcactgaggccgcccgggcgtcggg
cgacctttggtcgcccggcctcagtgagcgagcgagcgcgcagagagggagtggccaactccatcactaggggttcctgcggccgca
cgcgtactagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaagagctcgcgatccgccaccatgctgggcaacggatctgctatcgttcctatcgacc
agtgcttctgcctggcttggaccgactctctgggctctgataccgaacagctggtggccaatatcctgcagtggttcgccttcggcttcagc
attctgatcctgatgttctacgcctaccagacctggcgggccacctgtggctgggaggaagtgtatgtgtgctgcgtggaactgacaaagg
tgatcatcgagttcttccacgagttcgacgaccccagcatgctgtacctggccaatggcgagagagtgcagtggctgcggtacgccgagt
ggctgctgacctgtcctgtcatctgcatccacctgtccaacctgaccggcctgaaggacgattacagcaaaagaaccatgagactgctcgt
gtccgatgtgggcaccatcgtgtggggcgctaccagcgctatgagcaccggctacgtgaaggttatcttctttgtgctgggttgcatctatg
gcgccaacactttcttccacgccgccaaggtgtacatcgagagctaccacgtggtccccaagggcagacctagaacagtggtgcggatc
atggcctggctgtttttcctgagctggggaatgtttccagtgctgttcgtggtgggccctgagggatttgatgccatcagcgtgtacggcag
cacaatcggccatacaatcattgacctgatgagcaagaactgttggggcctgctgggacactacttgagggtgctgatccaccagcacat
catcatctacggcgacatcagaaagaccaccaaaatcaacgtggccggagaagaaatggaagtcgagacaatggtggaccaagagga
cgaggaaacagtggaattcggaggcggaggtggagctagcaaaggagaagaactcttcactggagttgtcccaattcttgttgaattaga
tggtgatgttaacggccacaagttctctgtcagtggagagggtgaaggtgatgcaacatacggaaaacttaccctgaagttcatctgcacta
ctggcaaactgcctgttccatggccaacactagtcactactctgtgctatggtgttcaatgcttttcaagatacccggatcatatgaaacggc
atgactttttcaagagtgccatgcccgaaggttatgtacaggaaaggaccatcttcttcaaagatgacggcaactacaagacacgtgctga
agtcaagtttgaaggtgatacccttgttaatagaatcgagttaaaaggtattgacttcaaggaagatggcaacattctgggacacaaattgga
atacaactataactcacacaatgtatacatcatggcagacaaacaaaagaatggaatcaaagtgaacttcaagacccgccacaacattgaa
gatggaagcgttcaactagcagaccattatcaacaaaatactccaattggcgatggccctgtccttttaccagacaaccattacctgtccac
acaatctgccctttcgaaagatcccaacgaaaagagagaccacatggtccttcttgagtttgtaacagctgctgggattacacatggcatgg
atgaactgtacaactaataactcgagggtaaccgtggtaccgatctgatgcggggatccggtaacgtcgagagatctacgggtggcatcc
ctgtgacccctccccagtgcctctcctggccctggaagttgccactccagtgcccaccagccttgtcctaataaaattaagttgcatcattttg
tctgactaggtgtccttctataatattatggggtggaggggggtggtatggagcaaggggcaagttgggaagacaacctgtagggcctgc
ggggtctattgggaaccaagctggagtgcagtggcacaatcttggctcactgcaatctccgcctcctgggttcaagcgattctcctgcctca
gcctcccgagttgttgggattccaggcatgcatgaccaggctcagctaatttttgtttttttggtagagacggggtttcaccatattggccagg
ctggtctccaactcctaatctcaggtgatctacccaccttggcctcccaaattgctgggattacaggcgtgaaccactgctcccttccctgtc
cttctgattttgtaggtaaccacgtgcggaccgagcggccgcaggaacccctagtgatggagttggccactccctctctgcgcgctcgctc
gctcactgaggccgggcgaccaaaggtcgcccgacgcccgggctttgcccgggcggcctcagtgagcgagcgagcgcgcagctgcc
tgcaggtctgagacaataaccctgataaatgcttcaataatgtaagcttgtcgagaagtactagaggatcataatcagccataccacatttgt
agaggttttacttgctttaaaaaacctcccacacctccccctgaacctgaaacataaaatgaatgcaattgttgttgttaacttgtttattgcagc
ttataatggttacaaataaagcaatagcatcacaaatttcacaaataaagcatttttttcactgcattctagttgtggtttgtccaaactcatcaat
gtatcttatcatgtctggatctgatcactgatatcgcctaggagatccgaaccagataagtgaaatctagttccaaactattttgtcatttttaatt
ttcgtattagcttacgacgctacacccagttcccatctattttgtcactcttccctaaataatccttaaaaactccatttccacccctcccagttcc
caactattttgtccgcccacagcggggcatttttcttcctgttatgtttttaatcaaacatcctgccaactccatgtgacaaaccgtcatcttcgg
ctactttttctctgtcacagaatgaaaatttttctgtcatctcttcgttattaatgtttgtaattgactgaatatcaacgcttatttgcagcctgaatgg
cgaatg
SEQ ID NO: 21 pCAG-Chrown-mWPRE-hGHpA
gacgcgccctgtagcggcgcattaagcgcggcgggtgtggtggttacgcgcagcgtgaccgctacacttgccagcgcccta
gcgcccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttcgccc
tttgacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgtttacaatttcaggtg
gcacttttcggggaaatgtgcgcggaacccctatttgtttatttttctaaatacattcaaatatgtatccgctcatgagacaataaccctgataaa
tgcttcaataatattgaaaaaggaagagtatgagtattcaacatttccgtgtcgcccttattcccttttttgcggcattttgccttcctgtttttgctc
acccagaaacgctggtgaaagtaaaagatgctgaagatcagttgggtgcacgagtgggttacatcgaactggatctcaacagcggtaag
atccttgagagttttcgccccgaagaacgttttccaatgatgagcacttttaaagttctgctatgtggcgcggtattatcccgtattgacgccg
ggcaagagcaactcggtcgccgcatacactattctcagaatgacttggttgagtactcaccagtcacagaaaagcatcttacggatggcat
gacagtaagagaattatgcagtgctgccataaccatgagtgataacactgcggccaacttacttctgacaacgatcggaggaccgaagga
gctaaccgcttttttgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttcccggcaacaattaat
agactggatggaggcggataaagttgcaggaccacttctgcgctcggcccttccggctggctggtttattgctgataaatctggagccggt
gagcgtgggtctcgcggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaagtttactcatatatacttt
agattgatttaaaacttcatttttaatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgcttgcaaacaaaaaaacca
ccgctaccagcggtggtttgtttgccggatcaagagctaccaactctttttccgaaggtaactggcttcagcagagcgcagataccaaatac
tgtccttctagtgtagccgtagttaggccaccacttcaagaactctgtagcaccgcctacatacctcgctctgctaatcctgttaccagtggct
gctgccagtggcgataagtcgtgtcttaccgggttggactcaagacgatagttaccggataaggcgcagcggtcgggctgaacggggg
gttcgtgcacacagcccagcttggagcgaacgacctacaccgaactgagatacctacagcgtgagcattgagaaagcgccacgcttccc
gaagggagaaaggcggacaggtatccggtaagcggcagggtcggaacaggagagcgcacgagggagcttccagggggaaacgcc
tggtatctttatagtcctgtcgggtttcgccacctctgacttgagcgtcgatttttgtgatgctcgtcaggggggcggagcctatggaaaaac
gccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgc
ctgatgcggtattttctccttacgcatctgtgcggtatttcacaccgcagaccagccgcgtaacctggcaaaatcggttacggttgagtaata
aatggatgccctgcgtaagcgggtgtgggcggacaataaagtcttaaactgaacaaaatagatctaaactatgacaataaagtcttaaact
agacagaatagttgtaaactgaaatcagtccagttatgctgtgaaaaagcatactggacttttgttatggctaaagcaaactcttcattttctga
agtgcaaattgcccgtcgtattaaagaggggcgtggccaagggcatggtaaagactatattcgcggcgttgtgacaatttaccgaacaact
ccgcggccgggaagccgatctcggcttgaacgaattgttaggtggcggtacttgggtcgatatcaaagtgcatcacttcttcccgtatgcc
caactttgtatagagagccactgcgggatcgtcaccgtaatctgcttgcacgtagatcacataagcaccaagcgcgttggcctcatgcttga
ggagattgatgagcgcggtggcaatgccctgcctccggtgctcgccggagactgcgagatcatagatatagatctcactacgcggctgct
caaacctgggcagaacgtaagccgcgagagcgccaacaaccgcttcttggtcgaaggcagcaagcgcgatgaatgtcttactacggag
caagttcccgaggtaatcggagtccggctgatgttgggagtaggtggctacgtctccgaactcacgaccgaaaagatcaagagcagccc
gcatggatttgacttggtcagggccgagcctacatgtgcgaatgatgcccatacttgagccacctaactttgttttagggcgactgccctgct
gcgtaacatcgttgctgctgcgtaacatcgttgctgctccataacatcaaacatcgacccacggcgtaacgcgcttgctgcttggatgcccg
aggcatagactgtacaaaaaaacagtcataacaagccatgaaaaccgccactgcgccgttaccaccgctgcgttcggtcaaggttctgga
ccagttgcgtgagcgcatacgctacttgcattacagtttacgaaccgaacaggcttatgtcaactgggttcgtgccttcatccgtttccacgg
tgtgcgtcacccggcaaccttgggcagcagcgaagtcgaggcatttctgtcctggctggcgaacgagcgcaaggtttcggtctccacgc
atcgtcaggcattggcggccttgctgttcttctacggcaaggtgctgtgcacggatctgccctggcttcaggagatcggaagacctcggcc
gtcgcggcgcttgccggtggtgctgaccccggatgaagtggttcgcatcctcggttttctggaaggcgagcatcgtttgttcgcccaggac
tctagctatagttctagtggttggctacagcttgcatgcctgcaggcagctgcgcgctcgctcgctcactgaggccgcccgggcgtcggg
cgacctttggtcgcccggcctcagtgagcgagcgagcgcgcagagagggagtggccaactccatcactaggggttcctgcggccgca
cgcgtactagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaagagctcgcgatccgccaccatgctgggcaacggatctgctatcgttcctatcgacc
agtgcttctgcctggcttggaccgactctctgggctctgataccgaacagctggtggccaatatcctgcagtggttcgccttcggcttcagc
attctgatcctgatgttctacgcctaccagacctggcgggccacctgtggctgggaggaagtgtatgtgtgctgcgtggaactgacaaagg
tgatcatcgagttcttccacgagttcgacgaccccagcatgctgtacctggccaatggcgagagagtgcagtggctgcggtacgccgagt
ggctgctgacctgtcctgtcatctgcatccacctgtccaacctgaccggcctgaaggacgattacagcaaaagaaccatgagactgctcgt
gtccgatgtgggcaccatcgtgtggggcgctaccagcgctatgagcaccggctacgtgaaggttatcttctttgtgctgggttgcatctatg
gcgccaacactttcttccacgccgccaaggtgtacatcgagagctaccacgtggtccccaagggcagacctagaacagtggtgcggatc
atggcctggctgtttttcctgagctggggaatgtttccagtgctgttcgtggtgggccctgagggatttgatgccatcagcgtgtacggcag
cacaatcggccatacaatcattgacctgatgagcaagaactgttggggcctgctgggacactacttgagggtgctgatccaccagcacat
catcatctacggcgacatcagaaagaccaccaaaatcaacgtggccggagaagaaatggaagtcgagacaatggtggaccaagagga
cgaggaaacagtgtaataactcgagggtaaccgtggtaccgataatcaacctctggattacaaaatttgtgaaagattgactggtattcttaa
ctatgttgctccttttacgctatgtggatacgctgctttaatgcctttgtatcatgctattgcttcccgtatggctttcattttctcctccttgtataaat
cctggttgctgtctctttatgaggagttgtggcccgttgtcaggcaacgtggcgtggtgtgcactgtgtttgctgacgcaacccccactggtt
ggggcattgccaccacctgtcagctcctttccgggactttcgctttccccctccctattgccacggcggaactcatcgccgcctgccttgcc
cgctgctggacaggggctcggctgttgggcactgacaattccgtggtgttgtcggggaaatcatcgtcctttccttggctgctcgcctgtgtt
gccacctggattctgcgcgggacgtccttctgctacgtcccttcggccctcaatccagcggaccttccttcccgcggcctgctgccggctc
tgcggcctcttccgcgtcttcgccttcgccctcagacgagtcggatctccctttgggccgcctccccgcctgatgcggggatccggtaacg
tcgagagatctacgggtggcatccctgtgacccctccccagtgcctctcctggccctggaagttgccactccagtgcccaccagccttgtc
ctaataaaattaagttgcatcattttgtctgactaggtgtccttctataatattatggggtggaggggggtggtatggagcaaggggcaagtt
gggaagacaacctgtagggcctgcggggtctattgggaaccaagctggagtgcagtggcacaatcttggctcactgcaatctccgcctc
ctgggttcaagcgattctcctgcctcagcctcccgagttgttgggattccaggcatgcatgaccaggctcagctaatttttgtttttttggtaga
gacggggtttcaccatattggccaggctggtctccaactcctaatctcaggtgatctacccaccttggcctcccaaattgctgggattacag
gcgtgaaccactgctcccttccctgtccttctgattttgtaggtaaccacgtgcggaccgagcggccgcaggaacccctagtgatggagtt
ggccactccctctctgcgcgctcgctcgctcactgaggccgggcgaccaaaggtcgcccgacgcccgggctttgcccgggcggcctca
gtgagcgagcgagcgcgcagctgcctgcaggtctgagacaataaccctgataaatgcttcaataatgtaagcttgtcgagaagtactaga
ggatcataatcagccataccacatttgtagaggttttacttgctttaaaaaacctcccacacctccccctgaacctgaaacataaaatgaatg
caattgttgttgttaacttgtttattgcagcttataatggttacaaataaagcaatagcatcacaaatttcacaaataaagcatttttttcactgcat
tctagttgtggtttgtccaaactcatcaatgtatcttatcatgtctggatctgatcactgatatcgcctaggagatccgaaccagataagtgaaa
tctagttccaaactattttgtcatttttaattttcgtattagcttacgacgctacacccagttcccatctattttgtcactcttccctaaataatcctta
aaaactccatttccacccctcccagttcccaactattttgtccgcccacagcggggcatttttcttcctgttatgtttttaatcaaacatcctgcc
aactccatgtgacaaaccgtcatcttcggctactttttctctgtcacagaatgaaaatttttctgtcatctcttcgttattaatgtttgtaattgactg
aatatcaacgcttatttgcagcctgaatggcgaatg
SEQ ID NO: 22 pChrown-tdTomato-hGHpA
gacgcgccctgtagcggcgcattaagcgcggcgggtgtggtggttacgcgcagcgtgaccgctacacttgccagcgcccta
gcgcccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttcgccc
tttgacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgtttacaatttcaggtg
gcacttttcggggaaatgtgcgcggaacccctatttgtttatttttctaaatacattcaaatatgtatccgctcatgagacaataaccctgataaa
tgcttcaataatattgaaaaaggaagagtatgagtattcaacatttccgtgtcgcccttattcccttttttgcggcattttgccttcctgtttttgctc
acccagaaacgctggtgaaagtaaaagatgctgaagatcagttgggtgcacgagtgggttacatcgaactggatctcaacagcggtaag
atccttgagagttttcgccccgaagaacgttttccaatgatgagcacttttaaagttctgctatgtggcgcggtattatcccgtattgacgccg
ggcaagagcaactcggtcgccgcatacactattctcagaatgacttggttgagtactcaccagtcacagaaaagcatcttacggatggcat
gacagtaagagaattatgcagtgctgccataaccatgagtgataacactgcggccaacttacttctgacaacgatcggaggaccgaagga
gctaaccgcttttttgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttcccggcaacaattaat
agactggatggaggcggataaagttgcaggaccacttctgcgctcggcccttccggctggctggtttattgctgataaatctggagccggt
gagcgtgggtctcgcggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaagtttactcatatatacttt
agattgatttaaaacttcatttttaatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgcttgcaaacaaaaaaacca
ccgctaccagcggtggtttgtttgccggatcaagagctaccaactctttttccgaaggtaactggcttcagcagagcgcagataccaaatac
tgtccttctagtgtagccgtagttaggccaccacttcaagaactctgtagcaccgcctacatacctcgctctgctaatcctgttaccagtggct
gctgccagtggcgataagtcgtgtcttaccgggttggactcaagacgatagttaccggataaggcgcagcggtcgggctgaacggggg
gttcgtgcacacagcccagcttggagcgaacgacctacaccgaactgagatacctacagcgtgagcattgagaaagcgccacgcttccc
gaagggagaaaggcggacaggtatccggtaagcggcagggtcggaacaggagagcgcacgagggagcttccagggggaaacgcc
tggtatctttatagtcctgtcgggtttcgccacctctgacttgagcgtcgatttttgtgatgctcgtcaggggggcggagcctatggaaaaac
gccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgc
ctgatgcggtattttctccttacgcatctgtgcggtatttcacaccgcagaccagccgcgtaacctggcaaaatcggttacggttgagtaata
aatggatgccctgcgtaagcgggtgtgggcggacaataaagtcttaaactgaacaaaatagatctaaactatgacaataaagtcttaaact
agacagaatagttgtaaactgaaatcagtccagttatgctgtgaaaaagcatactggacttttgttatggctaaagcaaactcttcattttctga
agtgcaaattgcccgtcgtattaaagaggggcgtggccaagggcatggtaaagactatattcgcggcgttgtgacaatttaccgaacaact
ccgcggccgggaagccgatctcggcttgaacgaattgttaggtggcggtacttgggtcgatatcaaagtgcatcacttcttcccgtatgcc
caactttgtatagagagccactgcgggatcgtcaccgtaatctgcttgcacgtagatcacataagcaccaagcgcgttggcctcatgcttga
ggagattgatgagcgcggtggcaatgccctgcctccggtgctcgccggagactgcgagatcatagatatagatctcactacgcggctgct
caaacctgggcagaacgtaagccgcgagagcgccaacaaccgcttcttggtcgaaggcagcaagcgcgatgaatgtcttactacggag
caagttcccgaggtaatcggagtccggctgatgttgggagtaggtggctacgtctccgaactcacgaccgaaaagatcaagagcagccc
gcatggatttgacttggtcagggccgagcctacatgtgcgaatgatgcccatacttgagccacctaactttgttttagggcgactgccctgct
gcgtaacatcgttgctgctgcgtaacatcgttgctgctccataacatcaaacatcgacccacggcgtaacgcgcttgctgcttggatgcccg
aggcatagactgtacaaaaaaacagtcataacaagccatgaaaaccgccactgcgccgttaccaccgctgcgttcggtcaaggttctgga
ccagttgcgtgagcgcatacgctacttgcattacagtttacgaaccgaacaggcttatgtcaactgggttcgtgccttcatccgtttccacgg
tgtgcgtcacccggcaaccttgggcagcagcgaagtcgaggcatttctgtcctggctggcgaacgagcgcaaggtttcggtctccacgc
atcgtcaggcattggcggccttgctgttcttctacggcaaggtgctgtgcacggatctgccctggcttcaggagatcggaagacctcggcc
gtcgcggcgcttgccggtggtgctgaccccggatgaagtggttcgcatcctcggttttctggaaggcgagcatcgtttgttcgcccaggac
tctagctatagttctagtggttggctacagcttgcatgcctgcaggcagctgcgcgctcgctcgctcactgaggccgcccgggcgtcggg
cgacctttggtcgcccggcctcagtgagcgagcgagcgcgcagagagggagtggccaactccatcactaggggttcctgcggccgca
cgcgtctagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccattgacgtc
aatgggtggagtatttacggtaaactgcccacttggcagtacatcaagtgtatcatatgccaagtacgccccctattgacgtcaatgacggt
aaatggcccgcctggcattatgcccagtacatgaccttatgggactttcctacttggcagtacatctacgtattagtcatcgctattaccatgg
tcgaggtgagccccacgttctgcttcactctccccatctcccccccctccccacccccaattttgtatttatttattttttaattattttgtgcagcg
atgggggcggggggggggggggggcgcgcgccaggcggggggggcggggcgaggggcggggcggggcgaggcggagagg
tgcggcggcagccaatcagagcggcgcgctccgaaagtttccttttatggcgaggcggcggcggcggcggccctataaaaagcgaag
cgcgcggcgggcgggagtcgctgcgcgctgccttcgccccgtgccccgctccgccgccgcctcgcgccgcccgccccggctctgact
gaccgcgttactcccacaggtgagcgggcgggacggcccttctcctccgggctgtaattagcgcttggtttaatgacggcttgtttcttttct
gtggctgcgtgaaagccttgaggggctccgggagggccctttgtgcggggggagcggctcggggggtgcgtgcgtgtgtgtgtgcgtg
gggagcgccgcgtgcggctccgcgctgcccggcggctgtgagcgctgcgggcgcggcgcggggctttgtgcgctccgcagtgtgcg
cgaggggagcgcggccgggggcggtgccccgcggtgcggggggggctgcgaggggaacaaaggctgcgtgcggggtgtgtgcgt
gggggggtgagcagggggtgtgggcgcgtcggtcgggctgcaaccccccctgcacccccctccccgagttgctgagcacggcccgg
cttcgggtgcggggctccgtacggggcgtggcgcggggctcgccgtgccgggcggggggtggcggcaggtgggggtgccgggcg
gggcggggccgcctcgggccggggagggctcgggggaggggcgcggcggcccccggagcgccggcggctgtcgaggcgcggc
gagccgcagccattgccttttatggtaatcgtgcgagagggcgcagggacttcctttgtcccaaatctgtgcggagccgaaatctgggag
gcgccgccgcaccccctctagcgggcgcggggcgaagcggtgcggcgccggcaggaaggaaatgggcggggagggccttcgtgc
gtcgccgcgccgccgtccccttctccctctccagcctcggggctgtccgcggggggacggctgccttcgggggggacggggcagggc
ggggttcggcttctggcgtgtgaccggcggctctagagcctctgctaaccatgttcatgccttcttctttttcctacagctcctgggcaacgtg
ctggttattgtgctgtctcatcattttggcaaagagctcgcgatccgccaccatgctgggcaacggatctgctatcgttcctatcgaccagtg
cttctgcctggcttggaccgactctctgggctctgataccgaacagctggtggccaatatcctgcagtggttcgccttcggcttcagcattct
gatcctgatgttctacgcctaccagacctggcgggccacctgtggctgggaggaagtgtatgtgtgctgcgtggaactgacaaaggtgat
catcgagttcttccacgagttcgacgaccccagcatgctgtacctggccaatggcgagagagtgcagtggctgcggtacgccgagtggc
tgctgacctgtcctgtcatctgcatccacctgtccaacctgaccggcctgaaggacgattacagcaaaagaaccatgagactgctcgtgtc
cgatgtgggcaccatcgtgtggggcgctaccagcgctatgagcaccggctacgtgaaggttatcttctttgtgctgggttgcatctatggcg
ccaacactttcttccacgccgccaaggtgtacatcgagagctaccacgtggtccccaagggcagacctagaacagtggtgcggatcatg
gcctggctgtttttcctgagctggggaatgtttccagtgctgttcgtggtgggccctgagggatttgatgccatcagcgtgtacggcagcac
aatcggccatacaatcattgacctgatgagcaagaactgttggggcctgctgggacactacttgagggtgctgatccaccagcacatcatc
atctacggcgacatcagaaagaccaccaaaatcaacgtggccggagaagaaatggaagtcgagacaatggtggaccaagaggacga
ggaaacagtggaattcgtagcagtgagtaagggcgaggaagtgatcaaagagttcatgcggtttaaggtgagaatggaaggaagcatg
aacggccacgagttcgaaattgagggagaaggagagggacggccctacgagggcacccagacagccaagctgaaagtgacaaagg
gcgggcctctgccattcgcttgggacatcctgagcccacagtttatgtacggctccaaggcctatgtgaaacatccagctgacattcccgat
tataagaaactgagcttccccgaggggtttaagtgggaaagagtgatgaacttcgaggacggaggcctggtgactgtgacccaggacag
ctccctgcaggatgggaccctgatctacaaggtgaaaatgagagggacaaattttccccctgatggacctgtgatgcagaagaaaactat
gggatgggaggcctccaccgaaaggctgtatccacgcgacggggtgctgaaaggagaaatccaccaggctctgaagctgaaagatgg
gggacattacctggtggagttcaagacaatctacatggccaagaaacctgtgcagctgccaggctactattacgtggacacaaaactgga
tatcacttcacacaacgaggactacactattgtggagcagtatgaacggagcgaggggagacaccatctgttcctgggccatgggactgg
aagtaccggctcagggtctagtggaaccgcctcaagcgaggataacaatatggctgtgatcaaagagttcatgaggtttaaggtgcgcat
ggagggcagcatgaatgggcacgaatttgagattgaaggagagggcgaagggaggccttacgagggcacacagactgccaagctga
aagtgaccaagggaggaccactgcctttcgcttgggatatcctgtctcctcagtttatgtacggaagtaaggcctatgtcaagcatcccgct
gacattcctgattacaagaaactgtctttcccagagggctttaagtgggagagagtgatgaattttgaagatggaggcctggtgaccgtgac
acaggactcctctctgcaggatggcactctgatctacaaagtcaaaatgcgcggcaccaattttccacccgatgggcccgtgatgcagaa
gaaaacaatggggtgggaggccagcactgaacggctgtatcctagagacggagtgctgaagggcgaaatccaccaggccctgaagct
gaaagacggcggccactacctggtggagttcaaaaccatctacatggccaagaaaccagtgcagctgcccggctattactatgtggaca
ccaagctggatatcacatcccacaatgaagactacaccattgtggaacagtatgagaggtctgaaggacgccaccatctgtttctgtacgg
catggatgagctgtataagtaataactcgagggtaaccgtggtaccgatctgatgcggggatccggtaacgtcgagagatctacgggtgg
catccctgtgacccctccccagtgcctctcctggccctggaagttgccactccagtgcccaccagccttgtcctaataaaattaagttgcatc
attttgtctgactaggtgtccttctataatattatggggtggaggggggtggtatggagcaaggggcaagttgggaagacaacctgtaggg
cctgcggggtctattgggaaccaagctggagtgcagtggcacaatcttggctcactgcaatctccgcctcctgggttcaagcgattctcct
gcctcagcctcccgagttgttgggattccaggcatgcatgaccaggctcagctaatttttgtttttttggtagagacggggtttcaccatattg
gccaggctggtctccaactcctaatctcaggtgatctacccaccttggcctcccaaattgctgggattacaggcgtgaaccactgctccctt
ccctgtccttctgattttgtaggtaaccacgtgcggaccgagcggccgcaggaacccctagtgatggagttggccactccctctctgcgcg
ctcgctcgctcactgaggccgggcgaccaaaggtcgcccgacgcccgggctttgcccgggcggcctcagtgagcgagcgagcgcgc
agctgcctgcaggtctgagacaataaccctgataaatgcttcaataatgtaagcttgtcgagaagtactagaggatcataatcagccatacc
acatttgtagaggttttacttgctttaaaaaacctcccacacctccccctgaacctgaaacataaaatgaatgcaattgttgttgttaacttgttta
ttgcagcttataatggttacaaataaagcaatagcatcacaaatttcacaaataaagcatttttttcactgcattctagttgtggtttgtccaaact
catcaatgtatcttatcatgtctggatctgatcactgatatcgcctaggagatccgaaccagataagtgaaatctagttccaaactattttgtcat
ttttaattttcgtattagcttacgacgctacacccagttcccatctattttgtcactcttccctaaataatccttaaaaactccatttccacccctccc
agttcccaactattttgtccgcccacagcggggcatttttcttcctgttatgtttttaatcaaacatcctgccaactccatgtgacaaaccgtcat
cttcggctactttttctctgtcacagaatgaaaatttttctgtcatctcttcgttattaatgtttgtaattgactgaatatcaacgcttatttgcagcct
gaatggcgaatg
SEQ ID NO: 23 pCAG-Chrown-hGHpA
gacgcgccctgtagcggcgcattaagcgcggcgggtgtggtggttacgcgcagcgtgaccgctacacttgccagcgcccta
gcgcccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttcgccc
tttgacgttggagtccacgttctttaatagtggactcttgttccaaactggaacaacactcaaccctatctcggtctattcttttgatttataaggg
attttgccgatttcggcctattggttaaaaaatgagctgatttaacaaaaatttaacgcgaattttaacaaaatattaacgtttacaatttcaggtg
gcacttttcggggaaatgtgcgcggaacccctatttgtttatttttctaaatacattcaaatatgtatccgctcatgagacaataaccctgataaa
tgcttcaataatattgaaaaaggaagagtatgagtattcaacatttccgtgtcgcccttattcccttttttgcggcattttgccttcctgtttttgctc
acccagaaacgctggtgaaagtaaaagatgctgaagatcagttgggtgcacgagtgggttacatcgaactggatctcaacagcggtaag
atccttgagagttttcgccccgaagaacgttttccaatgatgagcacttttaaagttctgctatgtggcgcggtattatcccgtattgacgccg
ggcaagagcaactcggtcgccgcatacactattctcagaatgacttggttgagtactcaccagtcacagaaaagcatcttacggatggcat
gacagtaagagaattatgcagtgctgccataaccatgagtgataacactgcggccaacttacttctgacaacgatcggaggaccgaagga
gctaaccgcttttttgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttcccggcaacaattaat
agactggatggaggcggataaagttgcaggaccacttctgcgctcggcccttccggctggctggtttattgctgataaatctggagccggt
gagcgtgggtctcgcggtatcattgcagcactggggccagatggtaagccctcccgtatcgtagttatctacacgacggggagtcaggca
actatggatgaacgaaatagacagatcgctgagataggtgcctcactgattaagcattggtaactgtcagaccaagtttactcatatatacttt
agattgatttaaaacttcatttttaatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgcttgcaaacaaaaaaacca
ccgctaccagcggtggtttgtttgccggatcaagagctaccaactctttttccgaaggtaactggcttcagcagagcgcagataccaaatac
tgtccttctagtgtagccgtagttaggccaccacttcaagaactctgtagcaccgcctacatacctcgctctgctaatcctgttaccagtggct
gctgccagtggcgataagtcgtgtcttaccgggttggactcaagacgatagttaccggataaggcgcagcggtcgggctgaacggggg
gttcgtgcacacagcccagcttggagcgaacgacctacaccgaactgagatacctacagcgtgagcattgagaaagcgccacgcttccc
gaagggagaaaggcggacaggtatccggtaagcggcagggtcggaacaggagagcgcacgagggagcttccagggggaaacgcc
tggtatctttatagtcctgtcgggtttcgccacctctgacttgagcgtcgatttttgtgatgctcgtcaggggggcggagcctatggaaaaac
gccagcaacgcggcctttttacggttcctggccttttgctggccttttgctcacatgttctttcctgcgttatcccctgattctgtggataaccgt
attaccgcctttgagtgagctgataccgctcgccgcagccgaacgaccgagcgcagcgagtcagtgagcgaggaagcggaagagcgc
ctgatgcggtattttctccttacgcatctgtgcggtatttcacaccgcagaccagccgcgtaacctggcaaaatcggttacggttgagtaata
aatggatgccctgcgtaagcgggtgtgggcggacaataaagtcttaaactgaacaaaatagatctaaactatgacaataaagtcttaaact
agacagaatagttgtaaactgaaatcagtccagttatgctgtgaaaaagcatactggacttttgttatggctaaagcaaactcttcattttctga
agtgcaaattgcccgtcgtattaaagaggggcgtggccaagggcatggtaaagactatattcgcggcgttgtgacaatttaccgaacaact
ccgcggccgggaagccgatctcggcttgaacgaattgttaggtggcggtacttgggtcgatatcaaagtgcatcacttcttcccgtatgcc
caactttgtatagagagccactgcgggatcgtcaccgtaatctgcttgcacgtagatcacataagcaccaagcgcgttggcctcatgcttga
ggagattgatgagcgcggtggcaatgccctgcctccggtgctcgccggagactgcgagatcatagatatagatctcactacgcggctgct
caaacctgggcagaacgtaagccgcgagagcgccaacaaccgcttcttggtcgaaggcagcaagcgcgatgaatgtcttactacggag
caagttcccgaggtaatcggagtccggctgatgttgggagtaggtggctacgtctccgaactcacgaccgaaaagatcaagagcagccc
gcatggatttgacttggtcagggccgagcctacatgtgcgaatgatgcccatacttgagccacctaactttgttttagggcgactgccctgct
gcgtaacatcgttgctgctgcgtaacatcgttgctgctccataacatcaaacatcgacccacggcgtaacgcgcttgctgcttggatgcccg
aggcatagactgtacaaaaaaacagtcataacaagccatgaaaaccgccactgcgccgttaccaccgctgcgttcggtcaaggttctgga
ccagttgcgtgagcgcatacgctacttgcattacagtttacgaaccgaacaggcttatgtcaactgggttcgtgccttcatccgtttccacgg
tgtgcgtcacccggcaaccttgggcagcagcgaagtcgaggcatttctgtcctggctggcgaacgagcgcaaggtttcggtctccacgc
atcgtcaggcattggcggccttgctgttcttctacggcaaggtgctgtgcacggatctgccctggcttcaggagatcggaagacctcggcc
gtcgcggcgcttgccggtggtgctgaccccggatgaagtggttcgcatcctcggttttctggaaggcgagcatcgtttgttcgcccaggac
tctagctatagttctagtggttggctacagcttgcatgcctgcaggcagctgcgcgctcgctcgctcactgaggccgcccgggcgtcggg
cgacctttggtcgcccggcctcagtgagcgagcgagcgcgcagagagggagtggccaactccatcactaggggttcctgcggccgca
cgcgtctagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccattgacgtc
aatgggtggagtatttacggtaaactgcccacttggcagtacatcaagtgtatcatatgccaagtacgccccctattgacgtcaatgacggt
aaatggcccgcctggcattatgcccagtacatgaccttatgggactttcctacttggcagtacatctacgtattagtcatcgctattaccatgg
tcgaggtgagccccacgttctgcttcactctccccatctcccccccctccccacccccaattttgtatttatttattttttaattattttgtgcagcg
atgggggcggggggggggggggggcgcgcgccaggcggggcggggcggggcgaggggcggggcggggcgaggcggagagg
tgcggcggcagccaatcagagcggcgcgctccgaaagtttccttttatggcgaggcggcggcggcggcggccctataaaaagcgaag
cgcgcggcgggcgggagtcgctgcgcgctgccttcgccccgtgccccgctccgccgccgcctcgcgccgcccgccccggctctgact
gaccgcgttactcccacaggtgagcgggcgggacggcccttctcctccgggctgtaattagcgcttggtttaatgacggcttgtttcttttct
gtggctgcgtgaaagccttgaggggctccgggagggccctttgtgcggggggagcggctcggggggtgcgtgcgtgtgtgtgtgcgtg
gggagcgccgcgtgcggctccgcgctgcccggcggctgtgagcgctgcgggcgcggcgcggggctttgtgcgctccgcagtgtgcg
cgaggggagcgcggccgggggcggtgccccgcggtgcggggggggctgcgaggggaacaaaggctgcgtgcggggtgtgtgcgt
gggggggtgagcagggggtgtgggcgcgtcggtcgggctgcaaccccccctgcacccccctccccgagttgctgagcacggcccgg
cttcgggtgcggggctccgtacggggcgtggcgcggggctcgccgtgccgggcggggggtggcggcaggtgggggtgccgggcg
gggcggggccgcctcgggccggggagggctcgggggaggggcgcggcggcccccggagcgccggcggctgtcgaggcgcggc
gagccgcagccattgccttttatggtaatcgtgcgagagggcgcagggacttcctttgtcccaaatctgtgcggagccgaaatctgggag
gcgccgccgcaccccctctagcgggcgcggggcgaagcggtgcggcgccggcaggaaggaaatgggcggggagggccttcgtgc
gtcgccgcgccgccgtccccttctccctctccagcctcggggctgtccgcggggggacggctgccttcgggggggacggggcagggc
ggggttcggcttctggcgtgtgaccggcggctctagagcctctgctaaccatgttcatgccttcttctttttcctacagctcctgggcaacgtg
ctggttattgtgctgtctcatcattttggcaaagagctcgcgatccgccaccatgctgggcaacggatctgctatcgttcctatcgaccagtg
cttctgcctggcttggaccgactctctgggctctgataccgaacagctggtggccaatatcctgcagtggttcgccttcggcttcagcattct
gatcctgatgttctacgcctaccagacctggcgggccacctgtggctgggaggaagtgtatgtgtgctgcgtggaactgacaaaggtgat
catcgagttcttccacgagttcgacgaccccagcatgctgtacctggccaatggcgagagagtgcagtggctgcggtacgccgagtggc
tgctgacctgtcctgtcatctgcatccacctgtccaacctgaccggcctgaaggacgattacagcaaaagaaccatgagactgctcgtgtc
cgatgtgggcaccatcgtgtggggcgctaccagcgctatgagcaccggctacgtgaaggttatcttctttgtgctgggttgcatctatggcg
ccaacactttcttccacgccgccaaggtgtacatcgagagctaccacgtggtccccaagggcagacctagaacagtggtgcggatcatg
gcctggctgtttttcctgagctggggaatgtttccagtgctgttcgtggtgggccctgagggatttgatgccatcagcgtgtacggcagcac
aatcggccatacaatcattgacctgatgagcaagaactgttggggcctgctgggacactacttgagggtgctgatccaccagcacatcatc
atctacggcgacatcagaaagaccaccaaaatcaacgtggccggagaagaaatggaagtcgagacaatggtggaccaagaggacga
ggaaacagtgtaataactcgagggtaaccgtggtaccgatctgatgcggggatccggtaacgtcgagagatctacgggtggcatccctgt
gacccctccccagtgcctctcctggccctggaagttgccactccagtgcccaccagccttgtcctaataaaattaagttgcatcattttgtctg
actaggtgtccttctataatattatggggtggaggggggtggtatggagcaaggggcaagttgggaagacaacctgtagggcctgcggg
gtctattgggaaccaagctggagtgcagtggcacaatcttggctcactgcaatctccgcctcctgggttcaagcgattctcctgcctcagcc
tcccgagttgttgggattccaggcatgcatgaccaggctcagctaatttttgtttttttggtagagacggggtttcaccatattggccaggctg
gtctccaactcctaatctcaggtgatctacccaccttggcctcccaaattgctgggattacaggcgtgaaccactgctcccttccctgtccttc
tgattttgtaggtaaccacgtgcggaccgagcggccgcaggaacccctagtgatggagttggccactccctctctgcgcgctcgctcgct
cactgaggccgggcgaccaaaggtcgcccgacgcccgggctttgcccgggcggcctcagtgagcgagcgagcgcgcagctgcctgc
aggtctgagacaataaccctgataaatgcttcaataatgtaagcttgtcgagaagtactagaggatcataatcagccataccacatttgtaga
ggttttacttgctttaaaaaacctcccacacctccccctgaacctgaaacataaaatgaatgcaattgttgttgttaacttgtttattgcagcttat
aatggttacaaataaagcaatagcatcacaaatttcacaaataaagcatttttttcactgcattctagttgtggtttgtccaaactcatcaatgtat
cttatcatgtctggatctgatcactgatatcgcctaggagatccgaaccagataagtgaaatctagttccaaactattttgtcatttttaattttcg
tattagcttacgacgctacacccagttcccatctattttgtcactcttccctaaataatccttaaaaactccatttccacccctcccagttcccaa
ctattttgtccgcccacagcggggcatttttcttcctgttatgtttttaatcaaacatcctgccaactccatgtgacaaaccgtcatcttcggcta
ctttttctctgtcacagaatgaaaatttttctgtcatctcttcgttattaatgtttgtaattgactgaatatcaacgcttatttgcagcctgaatggcg
aatg
SEQ ID NO: 24 500 base-pair mGluR6 promoter
ttaaaggcagtctaggggagaagcagacccagggagtcagagaggcagagagagaagagagcccttcctccactctcaag
ctctggagggggtctctgccctcaccctcatccctccccagaatccttaaatcctctagactgtagctctgattttacagctgtcacagactcg
tcctactagccagaggttggctcaggtaagcaccactggggaggtagcctagggtgcgctggggtgggtccagaggaagagctgccca
gaactgtgggggaaggagcgggaccgaccatcaacagggggacttttcagggagaatgagagcaatcctctggaggcctgggagag
gctgctgagttgctggtgcgcgagtcaccaacttttcctgcgctctcggtgtccggccagaatcccgaagtggcagctgagcacggggtg
gcagcttcgtccgccggctctcaaggcgtcccggtaacttcctttcccgcagtccaggagca
SEQ ID NO: 25 mGluR6 enhancer
gatctccagatggctaaacttttaaatcatgaatgaagtagatattaccaaattgctttttcagcatccatttagataatcatgttttttg
cctttaatctgttaatgtagtgaattacagaaatacatttcctaaatcattacatcccccaaatcgttaatctgctaaagtacatctctggctcaaa
caagactggttgt
SEQ ID NO: 26 mGluR6 intron 4
ggtgagtcccccaccccactcatcctccctgatgcttcctgtgtgggatgctcatttccacatttgtctcggagtcccacatgctga
gtaactctgagatttgctttaaaatgccatgcaggtaatttaaatgggaaggtctgatccaagtgatgaagtgcagccttgatagcatgcttcc
tccgccctcccacaggcttccatcttttgtggggtgcccacctccacaccttttcttttagctagagtggtcaagtggacaagctggtcattag
caatcaaggcgtttcagatctggaagtgggtggtgccattatggatcagtgagccctgtattttttgtgcctctgcacaaggtgggtagtgaa
gccctgtccattacataaccatggcatcccctagccatgacataaagggcagtgaaaaattctttaaggatgccagagctgctttttccattt
gtgtgtatgcgtgcaggtgtgtgttgtacatgacacaagtgtatgtgtgtgcatgtggaggcctgaggttgatttcaggaatcatcctcaattc
tttttctaccttattcactgaggcagggtctgtggagagatcaccgatatggctactgtgggattcccctgtctctgccttcagagccactcct
ggatacacagtacacctggctcagatggtcaccaccctcct
SEQ ID NO: 27 mGluR6 intron 3
actccaggccatgagcaactcctcacatctccctaagcccttcctgtcgccctctggagtcttttgttctgggaatgagacaggct
tgactggctgaaggttctccgggcctggcctgggaaacacaggaaaacacgactatttttattgttcattgtgggagagagaactggtagg
caaacccaagagcagaaaatgtaccgtgagggacactgccccagtaaaccctgaaacctacattatcctaagccagccaaggttcttttcc
agcctgggaagttgagcgtgacattggtggctgaatttgtagacagaatggcttctgagtgcccctgacattccccaaaaggaggctctct
gcattaatccatttgtctttattataataaaatatccaagtcagggcgttttttaaggaaaagacttatttttaacatcaactcttggaggtgaaag
ttcaggcagcgtgacaccagctctgctgaggacctagcttgcatcacattttgacaaatgttatggaaagagggagtagagaaggaaaga
gtgcatggagagaaggaacatcagaaagaagagggacagggttcactctttgatagctattcaccttcacagaattacctcacccttccag
aggtcaagagcaacatccccagtgacccaataaccttgcactaagccacacctcttttttattttttatgagacagggcctcactctgtatccc
tgaacttgctatgtggaccaagctgtcctcttgagtgctgggattaaaggcattcagtatcagggctgg
SEQ ID NO: 28 mGluR6 promoter
ggtgagtcccccaccccactcatcctccctgatgcttcctgtgtgggatgctcatttccacatttgtctcggagtcccacatgctga
gtaactctgagatttgctttaaaatgccatgcaggtaatttaaatgggaaggtctgatccaagtgatgaagtgcagccttgatagcatgcttcc
tccgccctcccacaggcttccatcttttgtggggtgcccacctccacaccttttcttttagctagagtggtcaagtggacaagctggtcattag
caatcaaggcgtttcagatctggaagtgggtggtgccattatggatcagtgagccctgtattttttgtgcctctgcacaaggtgggtagtgaa
gccctgtccattacataaccatggcatcccctagccatgacataaagggcagtgaaaaattctttaaggatgccagagctgctttttccattt
gtgtgtatgcgtgcaggtgtgtgttgtacatgacacaagtgtatgtgtgtgcatgtggaggcctgaggttgatttcaggaatcatcctcaattc
tttttctaccttattcactgaggcagggtctgtggagagatcaccgatatggctactgtgggattcccctgtctctgccttcagagccactcct
ggatacacagtacacctggctcagatggtcaccaccctcctagcgctcactccaggccatgagcaactcctcacatctccctaagcccttc
ctgtcgccctctggagtcttttgttctgggaatgagacaggcttgactggctgaaggttctccgggcctggcctgggaaacacaggaaaac
acgactatttttattgttcattgtgggagagagaactggtaggcaaacccaagagcagaaaatgtaccgtgagggacactgccccagtaaa
ccctgaaacctacattatcctaagccagccaaggttcttttccagcctgggaagttgagcgtgacattggtggctgaatttgtagacagaatg
gcttctgagtgcccctgacattccccaaaaggaggctctctgcattaatccatttgtctttattataataaaatatccaagtcagggcgtttttta
aggaaaagacttatttttaacatcaactcttggaggtgaaagttcaggcagcgtgacaccagctctgctgaggacctagcttgcatcacattt
tgacaaatgttatggaaagagggagtagagaaggaaagagtgcatggagagaaggaacatcagaaagaagagggacagggttcactc
tttgatagctattcaccttcacagaattacctcacccttccagaggtcaagagcaacatccccagtgacccaataaccttgcactaagccac
acctcttttttattttttatgagacagggcctcactctgtatccctgaacttgctatgtggaccaagctgtcctcttgagtgctgggattaaaggc
attcagtatcagggctggtttataatccggagatctccagatggctaaacttttaaatcatgaatgaagtagatattaccaaattgctttttcagc
atccatttagataatcatgttttttgcctttaatctgttaatgtagtgaattacagaaatacatttcctaaatcattacatcccccaaatcgttaatct
gctaaagtacatctctggctcaaacaagactggttgtgaccggtatttaaatttaaaggcagtctaggggagaagcagacccagggagtc
agagaggcagagagagaagagagcccttcctccactctcaagctctggagggggtctctgccctcaccctcatccctccccagaatcctt
aaatcctctagactgtagctctgattttacagctgtcacagactcgtcctactagccagaggttggctcaggtaagcaccactggggaggta
gcctagggtgcgctggggtgggtccagaggaagagctgcccagaactgtgggggaaggagcgggaccgaccatcaacagggggac
ttttcagggagaatgagagcaatcctctggaggcctgggagaggctgctgagttgctggtgcgcgagtcaccaacttttcctgcgctctcg
gtgtccggccagaatcccgaagtggcagctgagcacggggggcagcttcgtccgccggctctcaaggcgtcccggtaacttcctttcc
cgcagtccaggagca
SEQ ID NO: 29 ITR-CAG-Chrown-mWPRE-hGHpA-ITR
ctgcgcgctcgctcgctcactgaggccgcccgggcaaagcccgggcgtcgggcgacctttggtcgcccggcctcagtgagcgagcga
gcgcgcagagagggagtggccaactccatcactaggggttccttgcaggcctcctagggccgcacgcgtactagttattaatagtaatca
attacggggtcattagttcatagcccatatatggagttccgcgttacataacttacggtaaatggcccgcctggctgaccgcccaacgaccc
ccgcccattgacgtcaataatgacgtatgttcccatagtaacgccaatagggactttccattgacgtcaatgggtggagtatttacggtaaac
tgcccacttggcagtacatcaagtgtatcatatgccaagtacgccccctattgacgtcaatgacggtaaatggcccgcctggcattatgccc
agtacatgaccttatgggactttcctacttggcagtacatctacgtattagtcatcgctattaccatggtcgaggtgagccccacgttctgcttc
actctccccatctcccccccctccccacccccaattttgtatttatttattttttaattattttgtgcagcgatgggggcgggggggggggggg
ggcgcgcgccaggcggggcggggcggggcgaggggcggggcggggcgaggcggagaggtgcggcggcagccaatcagagcg
gcgcgctccgaaagtttccttttatggcgaggcggcggcggcggcggccctataaaaagcgaagcgcgcggcgggcgggagtcgctg
cgcgctgccttcgccccgtgccccgctccgccgccgcctcgcgccgcccgccccggctctgactgaccgcgttactcccacaggtgag
cgggcgggacggcccttctcctccgggctgtaattagcgcttggtttaatgacggcttgtttcttttctgtggctgcgtgaaagccttgaggg
gctccgggagggccctttgtgcggggggagcggctcggggggtgcgtgcgtgtgtgtgtgcgtggggagcgccgcgtgcggctccgc
gctgcccggcggctgtgagcgctgcgggcgcggcgcggggctttgtgcgctccgcagtgtgcgcgaggggagcgcggccgggggc
ggtgccccgcggtgcggggggggctgcgaggggaacaaaggctgcgtgcggggtgtgtgcgtgggggggtgagcagggggtgtg
ggcgcgtcggtcgggctgcaaccccccctgcacccccctccccgagttgctgagcacggcccggcttcgggtgcggggctccgtacg
gggcgtggcgcggggctcgccgtgccgggcggggggtggcggcaggtgggggtgccgggcggggcggggccgcctcgggccgg
ggagggctcgggggaggggcgcggcggcccccggagcgccggcggctgtcgaggcgcggcgagccgcagccattgccttttatgg
taatcgtgcgagagggcgcagggacttcctttgtcccaaatctgtgcggagccgaaatctgggaggcgccgccgcaccccctctagcgg
gcgcggggcgaagcggtgcggcgccggcaggaaggaaatgggcggggagggccttcgtgcgtcgccgcgccgccgtccccttctc
cctctccagcctcggggctgtccgcggggggacggctgccttcgggggggacggggcagggcggggttcggcttctggcgtgtgacc
ggcggctctagagcctctgctaaccatgttcatgccttcttctttttcctacagctcctgggcaacgtgctggttattgtgctgtctcatcattttg
gcaaagagctcgcgatccgccaccatgctgggcaacggatctgctatcgttcctatcgaccagtgcttctgcctggcttggaccgactctc
tgggctctgataccgaacagctggtggccaatatcctgcagtggttcgccttcggcttcagcattctgatcctgatgttctacgcctaccaga
cctggcgggccacctgtggctgggaggaagtgtatgtgtgctgcgtggaactgacaaaggtgatcatcgagttcttccacgagttcgacg
accccagcatgctgtacctggccaatggcgagagagtgcagtggctgcggtacgccgagtggctgctgacctgtcctgtcatctgcatcc
acctgtccaacctgaccggcctgaaggacgattacagcaaaagaaccatgagactgctcgtgtccgatgtgggcaccatcgtgtggggc
gctaccagcgctatgagcaccggctacgtgaaggttatcttctttgtgctgggttgcatctatggcgccaacactttcttccacgccgccaag
gtgtacatcgagagctaccacgtggtccccaagggcagacctagaacagtggtgcggatcatggcctggctgtttttcctgagctgggga
atgtttccagtgctgttcgtggtgggccctgagggatttgatgccatcagcgtgtacggcagcacaatcggccatacaatcattgacctgat
gagcaagaactgttggggcctgctgggacactacttgagggtgctgatccaccagcacatcatcatctacggcgacatcagaaagacca
ccaaaatcaacgtggccggagaagaaatggaagtcgagacaatggtggaccaagaggacgaggaaacagtgtaataactcgagggta
accgtggtaccgataatcaacctctggattacaaaatttgtgaaagattgactggtattcttaactatgttgctccttttacgctatgtggatacg
ctgctttaatgcctttgtatcatgctattgcttcccgtatggctttcattttctcctccttgtataaatcctggttgctgtctctttatgaggagttgtg
gcccgttgtcaggcaacgtggcgtggtgtgcactgtgtttgctgacgcaacccccactggttggggcattgccaccacctgtcagctccttt
ccgggactttcgctttccccctccctattgccacggcggaactcatcgccgcctgccttgcccgctgctggacaggggctcggctgttggg
cactgacaattccgtggtgttgtcggggaaatcatcgtcctttccttggctgctcgcctgtgttgccacctggattctgcgcgggacgtccttc
tgctacgtcccttcggccctcaatccagcggaccttccttcccgcggcctgctgccggctctgcggcctcttccgcgtcttcgccttcgccc
tcagacgagtcggatctccctttgggccgcctccccgcctgatgcggggatccggtaacgtcgagagatctacgggtggcatccctgtga
cccctccccagtgcctctcctggccctggaagttgccactccagtgcccaccagccttgtcctaataaaattaagttgcatcattttgtctgac
taggtgtccttctataatattatggggtggaggggggtggtatggagcaaggggcaagttgggaagacaacctgtagggcctgcggggt
ctattgggaaccaagctggagtgcagtggcacaatcttggctcactgcaatctccgcctcctgggttcaagcgattctcctgcctcagcctc
ccgagttgttgggattccaggcatgcatgaccaggctcagctaatttttgtttttttggtagagacggggtttcaccatattggccaggctggt
ctccaactcctaatctcaggtgatctacccaccttggcctcccaaattgctgggattacaggcgtgaaccactgctcccttccctgtccttctg
attttgtaggtaaccacgtgcggaccgagcggcctcgaggaggaacccctagtgatggagttggccactccctctctgcgcgctcgctcg
ctcactgaggccgggcgaccaaaggtcgcccgacgcccgggctttgcccgggcggcctcagtgagcgagcgagcgcgcag
SEQ ID NO: 30 mGluR6-Chrown-mWPRE-hGHpA
ggtgagtcccccaccccactcatcctccctgatgcttcctgtgtgggatgctcatttccacatttgtctcggagtcccacatgctga
gtaactctgagatttgctttaaaatgccatgcaggtaatttaaatgggaaggtctgatccaagtgatgaagtgcagccttgatagcatgcttcc
tccgccctcccacaggcttccatcttttgtggggtgcccacctccacaccttttcttttagctagagtggtcaagtggacaagctggtcattag
caatcaaggcgtttcagatctggaagtgggtggtgccattatggatcagtgagccctgtattttttgtgcctctgcacaaggtgggtagtgaa
gccctgtccattacataaccatggcatcccctagccatgacataaagggcagtgaaaaattctttaaggatgccagagctgctttttccattt
gtgtgtatgcgtgcaggtgtgtgttgtacatgacacaagtgtatgtgtgtgcatgtggaggcctgaggttgatttcaggaatcatcctcaattc
tttttctaccttattcactgaggcagggtctgtggagagatcaccgatatggctactgtgggattcccctgtctctgccttcagagccactcct
ggatacacagtacacctggctcagatggtcaccaccctcctagcgctcactccaggccatgagcaactcctcacatctccctaagcccttc
ctgtcgccctctggagtcttttgttctgggaatgagacaggcttgactggctgaaggttctccgggcctggcctgggaaacacaggaaaac
acgactatttttattgttcattgtgggagagagaactggtaggcaaacccaagagcagaaaatgtaccgtgagggacactgccccagtaaa
ccctgaaacctacattatcctaagccagccaaggttcttttccagcctgggaagttgagcgtgacattggtggctgaatttgtagacagaatg
gcttctgagtgcccctgacattccccaaaaggaggctctctgcattaatccatttgtctttattataataaaatatccaagtcagggcgtttttta
aggaaaagacttatttttaacatcaactcttggaggtgaaagttcaggcagcgtgacaccagctctgctgaggacctagcttgcatcacattt
tgacaaatgttatggaaagagggagtagagaaggaaagagtgcatggagagaaggaacatcagaaagaagagggacagggttcactc
tttgatagctattcaccttcacagaattacctcacccttccagaggtcaagagcaacatccccagtgacccaataaccttgcactaagccac
acctcttttttattttttatgagacagggcctcactctgtatccctgaacttgctatgtggaccaagctgtcctcttgagtgctgggattaaaggc
attcagtatcagggctggtttataatccggagatctccagatggctaaacttttaaatcatgaatgaagtagatattaccaaattgctttttcagc
atccatttagataatcatgttttttgcctttaatctgttaatgtagtgaattacagaaatacatttcctaaatcattacatcccccaaatcgttaatct
gctaaagtacatctctggctcaaacaagactggttgtgaccggtatttaaatttaaaggcagtctaggggagaagcagacccagggagtc
agagaggcagagagagaagagagcccttcctccactctcaagctctggagggggtctctgccctcaccctcatccctccccagaatcctt
aaatcctctagactgtagctctgattttacagctgtcacagactcgtcctactagccagaggttggctcaggtaagcaccactggggaggta
gcctagggtgcgctggggtgggtccagaggaagagctgcccagaactgtgggggaaggagcgggaccgaccatcaacagggggac
ttttcagggagaatgagagcaatcctctggaggcctgggagaggctgctgagttgctggtgcgcgagtcaccaacttttcctgcgctctcg
gtgtccggccagaatcccgaagtggcagctgagcacggggtggcagcttcgtccgccggctctcaaggcgtcccggtaacttcctttcc
cgcagtccaggagcagagctcgcgatccgccaccatgctgggcaacggatctgctatcgttcctatcgaccagtgcttctgcctggcttg
gaccgactctctgggctctgataccgaacagctggtggccaatatcctgcagtggttcgccttcggcttcagcattctgatcctgatgttcta
cgcctaccagacctggcgggccacctgtggctgggaggaagtgtatgtgtgctgcgtggaactgacaaaggtgatcatcgagttcttcca
cgagttcgacgaccccagcatgctgtacctggccaatggcgagagagtgcagtggctgcggtacgccgagtggctgctgacctgtcctg
tcatctgcatccacctgtccaacctgaccggcctgaaggacgattacagcaaaagaaccatgagactgctcgtgtccgatgtgggcacca
tcgtgtggggcgctaccagcgctatgagcaccggctacgtgaaggttatcttctttgtgctgggttgcatctatggcgccaacactttcttcc
acgccgccaaggtgtacatcgagagctaccacgtggtccccaagggcagacctagaacagtggtgcggatcatggcctggctgtttttcc
tgagctggggaatgtttccagtgctgttcgtggtgggccctgagggatttgatgccatcagcgtgtacggcagcacaatcggccatacaat
cattgacctgatgagcaagaactgttggggcctgctgggactctacttgagggtgctgatccaccagcacatcatcatctacggcgacatc
agaaagaccaccaaaatcaacgtggccggagaagaaatggaagtcgagacaatggtggaccaagaggacgaggaaacagtgtaata
actcgagggtaaccgtggtaccgataatcaacctctggattacaaaatttgtgaaagattgactggtattcttaactatgttgctccttttacgct
atgtggatacgctgctttaatgcctttgtatcatgctattgcttcccgtatggctttcattttctcctccttgtataaatcctggttgctgtctctttat
gaggagttgtggcccgttgtcaggcaacgtggcgtggtgtgcactgtgtttgctgacgcaacccccactggttggggcattgccaccacc
tgtcagctcctttccgggactttcgctttccccctccctattgccacggcggaactcatcgccgcctgccttgcccgctgctggacaggggc
tcggctgttgggcactgacaattccgtggtgttgtcggggaaatcatcgtcctttccttggctgctcgcctgtgttgccacctggattctgcgc
gggacgtccttctgctacgtcccttcggccctcaatccagcggaccttccttcccgcggcctgctgccggctctgcggcctcttccgcgtct
tcgccttcgccctcagacgagtcggatctccctttgggccgcctccccgcctgatgcggggatccggtaacgtcgagagatctacgggtg
gcatccctgtgacccctccccagtgcctctcctggccctggaagttgccactccagtgcccaccagccttgtcctaataaaattaagttgcat
cattttgtctgactaggtgtccttctataatattatggggtggaggggggtggtatggagcaaggggcaagttgggaagacaacctgtagg
gcctgcggggtctattgggaaccaagctggagtgcagtggcacaatcttggctcactgcaatctccgcctcctgggttcaagcgattctcc
tgcctcagcctcccgagttgttgggattccaggcatgcatgaccaggctcagctaatttttgtttttttggtagagacggggtttcaccatattg
gccaggctggtctccaactcctaatctcaggtgatctacccaccttggcctcccaaattgctgggattacaggcgtgaaccactgctccctt
ccctgtcctt
SEQ ID NO: 31 ITR1-mGluR6-Chrown-mWPRE-hGHpA-ITR2
ctgcgcgctcgctcgctcactgaggccgcccgggcaaagcccgggcgtcgggcgacctttggtcgcccggcctcagtgagc
gagcgagcgcgcagagagggagtggccaactccatcactaggggttccttgcaggcctcctagggccgcacgcgtacgcgtggtgag
tcccccaccccactcatcctccctgatgcttcctgtgtgggatgctcatttccacatttgtctcggagtcccacatgctgagtaactctgagatt
tgctttaaaatgccatgcaggtaatttaaatgggaaggtctgatccaagtgatgaagtgcagccttgatagcatgcttcctccgccctcccac
aggcttccatcttttgtggggtgcccacctccacaccttttcttttagctagagtggtcaagtggacaagctggtcattagcaatcaaggcgtt
tcagatctggaagtgggtggtgccattatggatcagtgagccctgtattttttgtgcctctgcacaaggtgggtagtgaagccctgtccatta
cataaccatggcatcccctagccatgacataaagggcagtgaaaaattctttaaggatgccagagctgctttttccatttgtgtgtatgcgtg
caggtgtgtgttgtacatgacacaagtgtatgtgtgtgcatgtggaggcctgaggttgatttcaggaatcatcctcaattctttttctaccttatt
cactgaggcagggtctgtggagagatcaccgatatggctactgtgggattcccctgtctctgccttcagagccactcctggatacacagta
cacctggctcagatggtcaccaccctcctagcgctcactccaggccatgagcaactcctcacatctccctaagcccttcctgtcgccctctg
gagtcttttgttctgggaatgagacaggcttgactggctgaaggttctccgggcctggcctgggaaacacaggaaaacacgactatttttat
tgttcattgtgggagagagaactggtaggcaaacccaagagcagaaaatgtaccgtgagggacactgccccagtaaaccctgaaaccta
cattatcctaagccagccaaggttcttttccagcctgggaagttgagcgtgacattggtggctgaatttgtagacagaatggcttctgagtgc
ccctgacattccccaaaaggaggctctctgcattaatccatttgtctttattataataaaatatccaagtcagggcgttttttaaggaaaagactt
atttttaacatcaactcttggaggtgaaagttcaggcagcgtgacaccagctctgctgaggacctagcttgcatcacattttgacaaatgttat
ggaaagagggagtagagaaggaaagagtgcatggagagaaggaacatcagaaagaagagggacagggttcactctttgatagctattc
accttcacagaattacctcacccttccagaggtcaagagcaacatccccagtgacccaataaccttgcactaagccacacctcttttttatttt
ttatgagacagggcctcactctgtatccctgaacttgctatgtggaccaagctgtcctcttgagtgctgggattaaaggcattcagtatcagg
gctggtttataatccggagatctccagatggctaaacttttaaatcatgaatgaagtagatattaccaaattgctttttcagcatccatttagata
atcatgttttttgcctttaatctgttaatgtagtgaattacagaaatacatttcctaaatcattacatcccccaaatcgttaatctgctaaagtacat
ctctggctcaaacaagactggttgtgaccggtatttaaatttaaaggcagtctaggggagaagcagacccagggagtcagagaggcaga
gagagaagagagcccttcctccactctcaagctctggagggggtctctgccctcaccctcatccctccccagaatccttaaatcctctaga
ctgtagctctgattttacagctgtcacagactcgtcctactagccagaggttggctcaggtaagcaccactggggaggtagcctagggtgc
gctggggtgggtccagaggaagagctgcccagaactgtgggggaaggagcgggaccgaccatcaacagggggacttttcagggaga
atgagagcaatcctctggaggcctgggagaggctgctgagttgctggtgcgcgagtcaccaacttttcctgcgctctcggtgtccggcca
gaatcccgaagtggcagctgagcacggggtggcagcttcgtccgccggctctcaaggcgtcccggtaacttcctttcccgcagtccagg
agcagagctcgcgatccgccaccatgctgggcaacggatctgctatcgttcctatcgaccagtgcttctgcctggcttggaccgactctct
gggctctgataccgaacagctggtggccaatatcctgcagtggttcgccttcggcttcagcattctgatcctgatgttctacgcctaccagac
ctggcgggccacctgtggctgggaggaagtgtatgtgtgctgcgtggaactgacaaaggtgatcatcgagttcttccacgagttcgacga
ccccagcatgctgtacctggccaatggcgagagagtgcagtggctgcggtacgccgagtggctgctgacctgtcctgtcatctgcatcca
cctgtccaacctgaccggcctgaaggacgattacagcaaaagaaccatgagactgctcgtgtccgatgtgggcaccatcgtgtggggcg
ctaccagcgctatgagcaccggctacgtgaaggttatcttctttgtgctgggttgcatctatggcgccaacactttcttccacgccgccaagg
tgtacatcgagagctaccacgtggtccccaagggcagacctagaacagtggtgcggatcatggcctggctgtttttcctgagctggggaa
tgtttccagtgctgttcgtggtgggccctgagggatttgatgccatcagcgtgtacggcagcacaatcggccatacaatcattgacctgatg
agcaagaactgttggggcctgctgggactctacttgagggtgctgatccaccagcacatcatcatctacggcgacatcagaaagaccacc
aaaatcaacgtggccggagaagaaatggaagtcgagacaatggtggaccaagaggacgaggaaacagtgtaataactcgagggtaac
cgtggtaccgataatcaacctctggattacaaaatttgtgaaagattgactggtattcttaactatgttgctccttttacgctatgtggatacgct
gctttaatgcctttgtatcatgctattgcttcccgtatggctttcattttctcctccttgtataaatcctggttgctgtctctttatgaggagttgtggc
ccgttgtcaggcaacgtggcgtggtgtgcactgtgtttgctgacgcaacccccactggttggggcattgccaccacctgtcagctcctttcc
gggactttcgctttccccctccctattgccacggcggaactcatcgccgcctgccttgcccgctgctggacaggggctcggctgttgggca
ctgacaattccgtggtgttgtcggggaaatcatcgtcctttccttggctgctcgcctgtgttgccacctggattctgcgcgggacgtccttctg
ctacgtcccttcggccctcaatccagcggaccttccttcccgcggcctgctgccggctctgcggcctcttccgcgtcttcgccttcgccctc
agacgagtcggatctccctttgggccgcctccccgcctgatgcggggatccggtaacgtcgagagatctacgggtggcatccctgtgac
ccctccccagtgcctctcctggccctggaagttgccactccagtgcccaccagccttgtcctaataaaattaagttgcatcattttgtctgact
aggtgtccttctataatattatggggtggaggggggtggtatggagcaaggggcaagttgggaagacaacctgtagggcctgcggggtc
tattgggaaccaagctggagtgcagtggcacaatcttggctcactgcaatctccgcctcctgggttcaagcgattctcctgcctcagcctcc
cgagttgttgggattccaggcatgcatgaccaggctcagctaatttttgtttttttggtagagacggggtttcaccatattggccaggctggtc
tccaactcctaatctcaggtgatctacccaccttggcctcccaaattgctgggattacaggcgtgaaccactgctcccttccctgtccttctga
ttttgtaggtaaccacgtgcggaccgagcggcctcgaggaggaacccctagtgatggagttggccactccctctctgcgcgctcgctcgc
tcactgaggccgggcgaccaaaggtcgcccgacgcccgggctttgcccgggcggcctcagtgagcgagcgagcgcgca
SEQ ID NO: 32 CAG-Chrown-GFP-mWPRE-hGHpA
ctagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaagagctcgcgatccgccaccatgctgggcaacggatctgctatcgttcctatcgacc
agtgcttctgcctggcttggaccgactctctgggctctgataccgaacagctggtggccaatatcctgcagtggttcgccttcggcttcagc
attctgatcctgatgttctacgcctaccagacctggcgggccacctgtggctgggaggaagtgtatgtgtgctgcgtggaactgacaaagg
tgatcatcgagttcttccacgagttcgacgaccccagcatgctgtacctggccaatggcgagagagtgcagtggctgcggtacgccgagt
ggctgctgacctgtcctgtcatctgcatccacctgtccaacctgaccggcctgaaggacgattacagcaaaagaaccatgagactgctcgt
gtccgatgtgggcaccatcgtgtggggcgctaccagcgctatgagcaccggctacgtgaaggttatcttctttgtgctgggttgcatctatg
gcgccaacactttcttccacgccgccaaggtgtacatcgagagctaccacgtggtccccaagggcagacctagaacagtggtgcggatc
atggcctggctgtttttcctgagctggggaatgtttccagtgctgttcgtggtgggccctgagggatttgatgccatcagcgtgtacggcag
cacaatcggccatacaatcattgacctgatgagcaagaactgttggggcctgctgggacactacttgagggtgctgatccaccagcacat
catcatctacggcgacatcagaaagaccaccaaaatcaacgtggccggagaagaaatggaagtcgagacaatggtggaccaagagga
cgaggaaacagtggaattcggaggcggaggtggagctagcaaaggagaagaactcttcactggagttgtcccaattcttgttgaattaga
tggtgatgttaacggccacaagttctctgtcagtggagagggtgaaggtgatgcaacatacggaaaacttaccctgaagttcatctgcacta
ctggcaaactgcctgttccatggccaacactagtcactactctgtgctatggtgttcaatgcttttcaagatacccggatcatatgaaacggc
atgactttttcaagagtgccatgcccgaaggttatgtacaggaaaggaccatcttcttcaaagatgacggcaactacaagacacgtgctga
agtcaagtttgaaggtgatacccttgttaatagaatcgagttaaaaggtattgacttcaaggaagatggcaacattctgggacacaaattgga
atacaactataactcacacaatgtatacatcatggcagacaaacaaaagaatggaatcaaagtgaacttcaagacccgccacaacattgaa
gatggaagcgttcaactagcagaccattatcaacaaaatactccaattggcgatggccctgtccttttaccagacaaccattacctgtccac
acaatctgccctttcgaaagatcccaacgaaaagagagaccacatggtccttcttgagtttgtaacagctgctgggattacacatggcatgg
atgaactgtacaactaataactcgagggtaaccgtggtaccgataatcaacctctggattacaaaatttgtgaaagattgactggtattcttaa
ctatgttgctccttttacgctatgtggatacgctgctttaatgcctttgtatcatgctattgcttcccgtatggctttcattttctcctccttgtataaat
cctggttgctgtctctttatgaggagttgtggcccgttgtcaggcaacgtggcgtggtgtgcactgtgtttgctgacgcaacccccactggtt
ggggcattgccaccacctgtcagctcctttccgggactttcgctttccccctccctattgccacggcggaactcatcgccgcctgccttgcc
cgctgctggacaggggctcggctgttgggcactgacaattccgtggtgttgtcggggaaatcatcgtcctttccttggctgctcgcctgtgtt
gccacctggattctgcgcgggacgtccttctgctacgtcccttcggccctcaatccagcggaccttccttcccgcggcctgctgccggctc
tgcggcctcttccgcgtcttcgccttcgccctcagacgagtcggatctccctttgggccgcctccccgcctgatgcggggatccggtaacg
tcgagagatctacgggtggcatccctgtgacccctccccagtgcctctcctggccctggaagttgccactccagtgcccaccagccttgtc
ctaataaaattaagttgcatcattttgtctgactaggtgtccttctataatattatggggtggaggggggtggtatggagcaaggggcaagtt
gggaagacaacctgtagggcctgcggggtctattgggaaccaagctggagtgcagtggcacaatcttggctcactgcaatctccgcctc
ctgggttcaagcgattctcctgcctcagcctcccgagttgttgggattccaggcatgcatgaccaggctcagctaatttttgtttttttggtaga
gacggggtttcaccatattggccaggctggtctccaactcctaatctcaggtgatctacccaccttggcctcccaaattgctgggattacag
gcgtgaaccactgctcccttccctgtcctt
SEQ ID NO: 33 CAG-Chrown-GFP-hGHpA
ctagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaagagctcgcgatccgccaccatgctgggcaacggatctgctatcgttcctatcgacc
agtgcttctgcctggcttggaccgactctctgggctctgataccgaacagctggtggccaatatcctgcagtggttcgccttcggcttcagc
attctgatcctgatgttctacgcctaccagacctggcgggccacctgtggctgggaggaagtgtatgtgtgctgcgtggaactgacaaagg
tgatcatcgagttcttccacgagttcgacgaccccagcatgctgtacctggccaatggcgagagagtgcagtggctgcggtacgccgagt
ggctgctgacctgtcctgtcatctgcatccacctgtccaacctgaccggcctgaaggacgattacagcaaaagaaccatgagactgctcgt
gtccgatgtgggcaccatcgtgtggggcgctaccagcgctatgagcaccggctacgtgaaggttatcttctttgtgctgggttgcatctatg
gcgccaacactttcttccacgccgccaaggtgtacatcgagagctaccacgtggtccccaagggcagacctagaacagtggtgcggatc
atggcctggctgtttttcctgagctggggaatgtttccagtgctgttcgtggtgggccctgagggatttgatgccatcagcgtgtacggcag
cacaatcggccatacaatcattgacctgatgagcaagaactgttggggcctgctgggacactacttgagggtgctgatccaccagcacat
catcatctacggcgacatcagaaagaccaccaaaatcaacgtggccggagaagaaatggaagtcgagacaatggtggaccaagagga
cgaggaaacagtggaattcggaggcggaggtggagctagcaaaggagaagaactcttcactggagttgtcccaattcttgttgaattaga
tggtgatgttaacggccacaagttctctgtcagtggagagggtgaaggtgatgcaacatacggaaaacttaccctgaagttcatctgcacta
ctggcaaactgcctgttccatggccaacactagtcactactctgtgctatggtgttcaatgcttttcaagatacccggatcatatgaaacggc
atgactttttcaagagtgccatgcccgaaggttatgtacaggaaaggaccatcttcttcaaagatgacggcaactacaagacacgtgctga
agtcaagtttgaaggtgatacccttgttaatagaatcgagttaaaaggtattgacttcaaggaagatggcaacattctgggacacaaattgga
atacaactataactcacacaatgtatacatcatggcagacaaacaaaagaatggaatcaaagtgaacttcaagacccgccacaacattgaa
gatggaagcgttcaactagcagaccattatcaacaaaatactccaattggcgatggccctgtccttttaccagacaaccattacctgtccac
acaatctgccctttcgaaagatcccaacgaaaagagagaccacatggtccttcttgagtttgtaacagctgctgggattacacatggcatgg
atgaactgtacaactaataactcgagggtaaccgtggtaccgatctgatgcggggatccggtaacgtcgagagatctacgggtggcatcc
ctgtgacccctccccagtgcctctcctggccctggaagttgccactccagtgcccaccagccttgtcctaataaaattaagttgcatcattttg
tctgactaggtgtccttctataatattatggggtggaggggggtggtatggagcaaggggcaagttgggaagacaacctgtagggcctgc
ggggtctattgggaaccaagctggagtgcagtggcacaatcttggctcactgcaatctccgcctcctgggttcaagcgattctcctgcctca
gcctcccgagttgttgggattccaggcatgcatgaccaggctcagctaatttttgtttttttggtagagacggggtttcaccatattggccagg
ctggtctccaactcctaatctcaggtgatctacccaccttggcctcccaaattgctgggattacaggcgtgaaccactgctcccttccctgtc
ctt
SEQ ID NO: 34 CAG-Chrown-mWPRE-hGHpA
ctagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaagagctcgcgatccgccaccatgctgggcaacggatctgctatcgttcctatcgacc
agtgcttctgcctggcttggaccgactctctgggctctgataccgaacagctggtggccaatatcctgcagtggttcgccttcggcttcagc
attctgatcctgatgttctacgcctaccagacctggcgggccacctgtggctgggaggaagtgtatgtgtgctgcgtggaactgacaaagg
tgatcatcgagttcttccacgagttcgacgaccccagcatgctgtacctggccaatggcgagagagtgcagtggctgcggtacgccgagt
ggctgctgacctgtcctgtcatctgcatccacctgtccaacctgaccggcctgaaggacgattacagcaaaagaaccatgagactgctcgt
gtccgatgtgggcaccatcgtgtggggcgctaccagcgctatgagcaccggctacgtgaaggttatcttctttgtgctgggttgcatctatg
gcgccaacactttcttccacgccgccaaggtgtacatcgagagctaccacgtggtccccaagggcagacctagaacagtggtgcggatc
atggcctggctgtttttcctgagctggggaatgtttccagtgctgttcgtggtgggccctgagggatttgatgccatcagcgtgtacggcag
cacaatcggccatacaatcattgacctgatgagcaagaactgttggggcctgctgggacactacttgagggtgctgatccaccagcacat
catcatctacggcgacatcagaaagaccaccaaaatcaacgtggccggagaagaaatggaagtcgagacaatggtggaccaagagga
cgaggaaacagtgtaataactcgagggtaaccgtggtaccgataatcaacctctggattacaaaatttgtgaaagattgactggtattcttaa
ctatgttgctccttttacgctatgtggatacgctgctttaatgcctttgtatcatgctattgcttcccgtatggctttcattttctcctccttgtataaat
cctggttgctgtctctttatgaggagttgtggcccgttgtcaggcaacgtggcgtggtgtgcactgtgtttgctgacgcaacccccactggtt
ggggcattgccaccacctgtcagctcctttccgggactttcgctttccccctccctattgccacggcggaactcatcgccgcctgccttgcc
cgctgctggacaggggctcggctgttgggcactgacaattccgtggtgttgtcggggaaatcatcgtcctttccttggctgctcgcctgtgtt
gccacctggattctgcgcgggacgtccttctgctacgtcccttcggccctcaatccagcggaccttccttcccgcggcctgctgccggctc
tgcggcctcttccgcgtcttcgccttcgccctcagacgagtcggatctccctttgggccgcctccccgcctgatgcggggatccggtaacg
tcgagagatctacgggtggcatccctgtgacccctccccagtgcctctcctggccctggaagttgccactccagtgcccaccagccttgtc
ctaataaaattaagttgcatcattttgtctgactaggtgtccttctataatattatggggtggaggggggtggtatggagcaaggggcaagtt
gggaagacaacctgtagggcctgcggggtctattgggaaccaagctggagtgcagtggcacaatcttggctcactgcaatctccgcctc
ctgggttcaagcgattctcctgcctcagcctcccgagttgttgggattccaggcatgcatgaccaggctcagctaatttttgtttttttggtaga
gacggggtttcaccatattggccaggctggtctccaactcctaatctcaggtgatctacccaccttggcctcccaaattgctgggattacag
gcgtgaaccactgctcccttccctgtcctt
SEQ ID NO: 35 Chrown-tdTomato-hGHpA
ctagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaagagctcgcgatccgccaccatgctgggcaacggatctgctatcgttcctatcgacc
agtgcttctgcctggcttggaccgactctctgggctctgataccgaacagctggtggccaatatcctgcagtggttcgccttcggcttcagc
attctgatcctgatgttctacgcctaccagacctggcgggccacctgtggctgggaggaagtgtatgtgtgctgcgtggaactgacaaagg
tgatcatcgagttcttccacgagttcgacgaccccagcatgctgtacctggccaatggcgagagagtgcagtggctgcggtacgccgagt
ggctgctgacctgtcctgtcatctgcatccacctgtccaacctgaccggcctgaaggacgattacagcaaaagaaccatgagactgctcgt
gtccgatgtgggcaccatcgtgtggggcgctaccagcgctatgagcaccggctacgtgaaggttatcttctttgtgctgggttgcatctatg
gcgccaacactttcttccacgccgccaaggtgtacatcgagagctaccacgtggtccccaagggcagacctagaacagtggtgcggatc
atggcctggctgtttttcctgagctggggaatgtttccagtgctgttcgtggtgggccctgagggatttgatgccatcagcgtgtacggcag
cacaatcggccatacaatcattgacctgatgagcaagaactgttggggcctgctgggacactacttgagggtgctgatccaccagcacat
catcatctacggcgacatcagaaagaccaccaaaatcaacgtggccggagaagaaatggaagtcgagacaatggtggaccaagagga
cgaggaaacagtggaattcgtagcagtgagtaagggcgaggaagtgatcaaagagttcatgcggtttaaggtgagaatggaaggaagc
atgaacggccacgagttcgaaattgagggagaaggagagggacggccctacgagggcacccagacagccaagctgaaagtgacaaa
gggcgggcctctgccattcgcttgggacatcctgagcccacagtttatgtacggctccaaggcctatgtgaaacatccagctgacattccc
gattataagaaactgagcttccccgaggggtttaagtgggaaagagtgatgaacttcgaggacggaggcctggtgactgtgacccagga
cagctccctgcaggatgggaccctgatctacaaggtgaaaatgagagggacaaattttccccctgatggacctgtgatgcagaagaaaac
tatgggatgggaggcctccaccgaaaggctgtatccacgcgacggggtgctgaaaggagaaatccaccaggctctgaagctgaaagat
gggggacattacctggtggagttcaagacaatctacatggccaagaaacctgtgcagctgccaggctactattacgtggacacaaaactg
gatatcacttcacacaacgaggactacactattgtggagcagtatgaacggagcgaggggagacaccatctgttcctgggccatgggact
ggaagtaccggctcagggtctagtggaaccgcctcaagcgaggataacaatatggctgtgatcaaagagttcatgaggtttaaggtgcgc
atggagggcagcatgaatgggcacgaatttgagattgaaggagagggcgaagggaggccttacgagggcacacagactgccaagctg
aaagtgaccaagggaggaccactgcctttcgcttgggatatcctgtctcctcagtttatgtacggaagtaaggcctatgtcaagcatcccgc
tgacattcctgattacaagaaactgtctttcccagagggctttaagtgggagagagtgatgaattttgaagatggaggcctggtgaccgtga
cacaggactcctctctgcaggatggcactctgatctacaaagtcaaaatgcgcggcaccaattttccacccgatgggcccgtgatgcaga
agaaaacaatggggtgggaggccagcactgaacggctgtatcctagagacggagtgctgaagggcgaaatccaccaggccctgaagc
tgaaagacggcggccactacctggtggagttcaaaaccatctacatggccaagaaaccagtgcagctgcccggctattactatgtggaca
ccaagctggatatcacatcccacaatgaagactacaccattgtggaacagtatgagaggtctgaaggacgccaccatctgtttctgtacgg
catggatgagctgtataagtaataactcgagggtaaccgtggtaccgatctgatgcggggatccggtaacgtcgagagatctacgggtgg
catccctgtgacccctccccagtgcctctcctggccctggaagttgccactccagtgcccaccagccttgtcctaataaaattaagttgcatc
attttgtctgactaggtgtccttctataatattatggggtggaggggggtggtatggagcaaggggcaagttgggaagacaacctgtaggg
cctgcggggtctattgggaaccaagctggagtgcagtggcacaatcttggctcactgcaatctccgcctcctgggttcaagcgattctcct
gcctcagcctcccgagttgttgggattccaggcatgcatgaccaggctcagctaatttttgtttttttggtagagacggggtttcaccatattg
gccaggctggtctccaactcctaatctcaggtgatctacccaccttggcctcccaaattgctgggattacaggcgtgaaccactgctccctt
ccctgtcctt
SEQ ID NO: 36 CAG-Chrown-hGHpA
ctagttattaatagtaatcaattacggggtcattagttcatagcccatatatggagttccgcgttacataacttacggtaaatggccc
gcctggctgaccgcccaacgacccccgcccattgacgtcaataatgacgtatgttcccatagtaacgccaatagggactttccattgacgt
caatgggtggagtatttacggtaaactgcccacttggcagtacatcaagtgtatcatatgccaagtacgccccctattgacgtcaatgacgg
taaatggcccgcctggcattatgcccagtacatgaccttatgggactttcctacttggcagtacatctacgtattagtcatcgctattaccatg
gtcgaggtgagccccacgttctgcttcactctccccatctcccccccctccccacccccaattttgtatttatttattttttaattattttgtgcagc
gatgggggcggggggggggggggggcgcgcgccaggcggggcggggcggggcgaggggcggggcggggcgaggcggagag
gtgcggcggcagccaatcagagcggcgcgctccgaaagtttccttttatggcgaggcggcggcggcggcggccctataaaaagcgaa
gcgcgcggcgggcgggagtcgctgcgcgctgccttcgccccgtgccccgctccgccgccgcctcgcgccgcccgccccggctctga
ctgaccgcgttactcccacaggtgagcgggcgggacggcccttctcctccgggctgtaattagcgcttggtttaatgacggcttgtttctttt
ctgtggctgcgtgaaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgctccgcagtgtg
cgcgaggggagcgcggccgggggcggtgccccgcggtgcggggggggctgcgaggggaacaaaggctgcgtgcggggtgtgtg
cgtgggggggtgagcagggggtgtgggcgcgtcggtcgggctgcaaccccccctgcacccccctccccgagttgctgagcacggcc
cggcttcgggtgcggggctccgtacggggcgtggcgcggggctcgccgtgccgggcggggggtggcggcaggtgggggtgccgg
gcggggcggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgcggagccgaaatctgg
gaggcgccgccgcaccccctctagcgggcgcggggcgaagcggtgcggcgccggcaggaaggaaatgggcggggagggccttcg
tgcgtcgccgcgccgccgtccccttctccctctccagcctcggggctgtccgcggggggacggctgccttcgggggggacggggcag
ggcggggttcggcttctggcgtgtgaccggcggctctagagcctctgctaaccatgttcatgccttcttctttttcctacagctcctgggcaa
cgtgctggttattgtgctgtctcatcattttggcaaagagctcgcgatccgccaccatgctgggcaacggatctgctatcgttcctatcgacc
agtgcttctgcctggcttggaccgactctctgggctctgataccgaacagctggtggccaatatcctgcagtggttcgccttcggcttcagc
attctgatcctgatgttctacgcctaccagacctggcgggccacctgtggctgggaggaagtgtatgtgtgctgcgtggaactgacaaagg
tgatcatcgagttcttccacgagttcgacgaccccagcatgctgtacctggccaatggcgagagagtgcagtggctgcggtacgccgagt
ggctgctgacctgtcctgtcatctgcatccacctgtccaacctgaccggcctgaaggacgattacagcaaaagaaccatgagactgctcgt
gtccgatgtgggcaccatcgtgtggggcgctaccagcgctatgagcaccggctacgtgaaggttatcttctttgtgctgggttgcatctatg
gcgccaacactttcttccacgccgccaaggtgtacatcgagagctaccacgtggtccccaagggcagacctagaacagtggtgcggatc
atggcctggctgtttttcctgagctggggaatgtttccagtgctgttcgtggtgggccctgagggatttgatgccatcagcgtgtacggcag
cacaatcggccatacaatcattgacctgatgagcaagaactgttggggcctgctgggacactacttgagggtgctgatccaccagcacat
catcatctacggcgacatcagaaagaccaccaaaatcaacgtggccggagaagaaatggaagtcgagacaatggtggaccaagagga
cgaggaaacagtgtaataactcgagggtaaccgtggtaccgatctgatgcggggatccggtaacgtcgagagatctacgggtggcatcc
ctgtgacccctccccagtgcctctcctggccctggaagttgccactccagtgcccaccagccttgtcctaataaaattaagttgcatcattttg
tctgactaggtgtccttctataatattatggggtggaggggggtggtatggagcaaggggcaagttgggaagacaacctgtagggcctgc
ggggtctattgggaaccaagctggagtgcagtggcacaatcttggctcactgcaatctccgcctcctgggttcaagcgattctcctgcctca
gcctcccgagttgttgggattccaggcatgcatgaccaggctcagctaatttttgtttttttggtagagacggggtttcaccatattggccagg
ctggtctccaactcctaatctcaggtgatctacccaccttggcctcccaaattgctgggattacaggcgtgaaccactgctcccttccctgtc
ctt
SEQ ID NO: 37 Wild-type AAV2 VP1 capsid protein
MAADGYLPDWLEDTLSEGIRQWWKLKPGPPPPKPAERHKDDSRGLVLPGYKYL
GPFNGLDKGEPVNEADAAALEHDKAYDRQLDSGDNPYLKYNHADAEFQERLKEDTSFG
GNLGRAVFQAKKRVLEPLGLVEEPVKTAPGKKRPVEHSPVEPDSSSGTGKAGQQPARKR
LNFGQTGDADSVPDPQPLGQPPAAPSGLGTNTMATGSGAPMADNNEGADGVGNSSGN
WHCDSTWMGDRVITTSTRTWALPTYNNHLYKQISSQSGASNDNHYFGYSTPWGYFDFN
RFHCHFSPRDWQRLINNNWGFRPKRLNFKLFNIQVKEVTQNDGTTTIANNLTSTVQVFT
DSEYQLPYVLGSAHQGCLPPFPADVFMVPQYGYLTLNNGSQAVGRSSFYCLEYFPSQML
RTGNNFTFSYTFEDVPFHSSYAHSQSLDRLMNPLIDQYLYYLSRTNTPSGTTTQSRLQFS
QAGASDIRDQSRNWLPGPCYRQQRVSKTSADNNNSEYSWTGATKYHLNGRDSLVNPGP
AMASHKDDEEKFFPQSGVLIFGKQGSEKTNVDIEKVMITDEEEIRTTNPVATEQYGSVST
NLQRGNRQAATADVNTQGVLPGMVWQDRDVYLQGPIWAKIPHTDGHFHPSPLMGGFG
LKHPPPQILIKNTPVPANPSTTFSAAKFASFITQYSTGQVSVEIEWELQKENSKRWNPEIQ
YTSNYNKSVNVDFTVDTNGVYSEPRPIGTRYLTRNL
SEQ ID NO: 38 AAV2 7m8 VP1 capsid protein
MAADGYLPDWLEDTLSEGIRQWWKLKPGPPPPKPAERHKDDSRGLVLPGYKYL
GPFNGLDKGEPVNEADAAALEHDKAYDRQLDSGDNPYLKYNHADAEFQERLKEDTSFG
GNLGRAVFQAKKRVLEPLGLVEEPVKTAPGKKRPVEHSPVEPDSSSGTGKAGQQPARKR
LNFGQTGDADSVPDPQPLGQPPAAPSGLGTNTMATGSGAPMADNNEGADGVGNSSGN
WHCDSTWMGDRVITTSTRTWALPTYNNHLYKQISSQSGASNDNHYFGYSTPWGYFDFN
RFHCHFSPRDWQRLINNNWGFRPKRLNFKLFNIQVKEVTQNDGTTTIANNLTSTVQVFT
DSEYQLPYVLGSAHQGCLPPFPADVFMVPQYGYLTLNNGSQAVGRSSFYCLEYFPSQML
RTGNNFTFSYTFEDVPFHSSYAHSQSLDRLMNPLIDQYLYYLSRTNTPSGTTTQSRLQFS
QAGASDIRDQSRNWLPGPCYRQQRVSKTSADNNNSEYSWTGATKYHLNGRDSLVNPGP
AMASHKDDEEKFFPQSGVLIFGKQGSEKTNVDIEKVMITDEEEIRTTNPVATEQYGSVST
NLQRGNLALGETTRPARQAATADVNTQGVLPGMVWQDRDVYLQGPIWAKIPHTDGHF
HPSPLMGGFGLKHPPPQILIKNTPVPANPSTTFSAAKFASFITQYSTGQVSVEIEWELQKE
NSKRWNPEIQYTSNYNKSVNVDFTVDTNGVYSEPRPIGTRYLTRNL

1. An infectious recombinant adeno-associated virus (rAAV) particle comprising
(i) a capsid protein having a modified amino acid sequence relative to a native AAV capsid of serotype 2, wherein the capsid protein comprises a substitution of tyrosine to phenylalanine at a position corresponding to position 444 of wild-type AAV2 VP1 capsid sequence; and
(ii) a vector genome consisting of a heterologous polynucleotide comprising from 5′ to 3′
an AAV2 inverted terminal repeat sequence (ITR1);
a promoter sequence selected from an mGluR6 promoter and a CAG promoter;
a polynucleotide sequence encoding a channelrhodopsin, wherein the channelrhodopsin comprises the amino acid sequence of SEQ ID NO: 5, or an amino acid sequence having at least 90% amino acid sequence identity thereto;
a polyadenylation sequence; and
an AAV2 inverted terminal repeat sequence (ITR2).

2. The rAAV particle of claim 1, wherein the heterologous polynucleotide comprises a woodchuck hepatitis virus posttranscriptional regulatory element (WPRE) between the polynucleotide sequence encoding the channelrhodopsin and the polyadenylation sequence.

3. (canceled)

4. (canceled)

5. The rAAV particle of claim 1, wherein the promoter is a CAG promoter.

6-11. (canceled)

12. The rAAV particle of claim 1, wherein the promoter comprises an mGluR6 promoter comprising from 3′ to 5′: intron 4 of an mGluR6 gene, intron 3 of the mGluR6 gene, an mGluR6enhancer, and a fragment of the mGluR6 promoter.

13-15. (canceled)

16. The rAAV particle of claim 1, wherein the polyadenylation signal is a human growth hormone polyadenylation sequence (hGHpA) or a Simian virus 40 polyadenylation sequence.

17. (canceled)

18. The rAAV particle of claim 1, wherein the heterologous polynucleotide comprises CAG-Chrown-mWPRE-hGHpA (SEQ ID NO: 34) or CAG-Chrown-hGHpA (SEQ ID NO: 36), or a nucleotide sequence having at least 99.5% nucleotide sequence identity to either of the foregoing.

19. The rAAV particle of claim 1, wherein the capsid protein comprises the amino acid sequence of SEQ ID NO: 7 or SEQ ID NO: 38.

20-32. (canceled)

33. An infectious recombinant adeno-associated virus (rAAV) particle comprising
(i) an AAV2 7m8 capsid protein, wherein the capsid protein comprises the amino acid sequence of SEQ ID NO: 7; and
(ii) a vector genome comprising a heterologous polynucleotide comprising from 5′ to 3′:
an AAV2 inverted terminal repeat sequence (ITR1) comprising SEQ ID NO: 15;
a CAG promoter comprising SEQ ID NO: 10;
a polynucleotide sequence encoding a channelrhodopsin having an amino acid sequence of SEQ ID NO: 5, or an amino acid sequence having at least 90% amino acid sequence identity thereto;
a woodchuck hepatitis virus posttranscriptional regulatory element (WPRE) comprising SEQ ID NO: 14, or a nucleotide sequence having at least 90% nucleotide sequence identity thereto;
a polyadenylation sequence comprising SEQ ID NO: 18; and
an AAV2 inverted terminal repeat sequence (ITR2) comprising SEQ ID NO: 16.

34. (canceled)

35. (canceled)

36. The rAAV particle of claim 33, wherein the heterologous polynucleotide does not encode a fluorescent protein or a sorting or targeting sequence.

37-42. (canceled)

43. The rAAV particle of claim 33, wherein the polynucleotide encoding the channelrhodopsin comprises SEQ ID NO: 17.

44-52. (canceled)

53. The rAAV particle of claim 33, wherein the heterologous polynucleotide comprises ITR-CAG-Chrown-mWPRE-hGHpA-ITR (SEQ ID NO: 29), or a nucleotide sequence having at least 99.5% nucleotide sequence identity thereto.

54-57. (canceled)

58. The rAAV particle of claim 1, wherein the heterologous polynucleotide comprises mGluR6-Chrown-mWPRE-hGHpA (SEQ ID NO: 30), or a nucleotide sequence having at least 99.5% nucleotide sequence identity thereto.

59. The rAAV particle of claim 60, wherein the heterologous polynucleotide comprises ITR1-mGluR6-Chrown-mWPRE-hGHpA-ITR2 (SEQ ID NO: 31), or a nucleotide sequence having at least 99.5% nucleotide sequence identity thereto.

60. An infectious recombinant adeno-associated virus (rAAV) particle comprising:
(i) an AAV2 7m8 capsid protein, wherein the capsid protein comprises SEQ ID NO: 7; and
(ii) a vector genome comprising a heterologous polynucleotide comprising from 5′ to 3′:
an AAV2 inverted terminal repeat sequence (ITR1) comprising SEQ ID NO: 15;
an mGluR6 promoter comprising SEQ ID NO:28;
a polynucleotide encoding a channelrhodopsin comprising the amino acid sequence of SEQ ID NO:5 or an amino acid sequence having at least 90% amino sequence identity thereto;
a woodchuck hepatitis virus posttranscriptional regulatory element (WPRE) comprising SEQ ID NO: 14 or a nucleotide sequence having at least 90% nucleotide sequence identity thereto;
a polyadenylation sequence comprising SEQ ID NO: 18; and
an AAV2 inverted terminal repeat sequence (ITR2) comprising SEQ ID NO:16.

61. (canceled)

62. A pharmaceutical composition comprising a plurality of the rAAV particles of claim 1, and a pharmaceutically acceptable carrier or excipient.

63. The pharmaceutical composition of claim 62, wherein the composition is formulated for intravitreal injection.

64. (canceled)

65. A method for treating a retinal disease in a human subject in need thereof, the method comprising administering to the human subject a therapeutically effective amount a pharmaceutical composition comprising a plurality of the rAAV particles of claim 1, wherein the plurality of rAAV particles is administered in one or more doses per eye.

66. The method of claim 65, wherein the plurality of rAAV particles is administered by intravitreal injection.

67. (canceled)

68. The method of claim 65, wherein the one or more doses each comprises about 1×10¹¹ viral genomes (vg).

69. The method of claim 65, wherein the one or more doses each comprises about 1×10⁸ to 1×10¹⁴ viral genomes (vg).

70. (canceled)

71. The method of claim 65, wherein the retinal disease is selected from Bardet-Biedl syndrome, chorioretinal atrophy or degeneration, cone or cone-rod dystrophy, congenital stationary night blindness, Leber congenital amaurosis (LCA), macular degeneration (MD), including age-related MD (AMD), ocular-retinal developmental disease, optic atrophy, retinitis pigmentosa, syndromic/systemic diseases with retinopathy, Usher syndrome, or other retinopathy, including diabetic retinopathy.

72. The method of claim 65, wherein the retinal disease is age-related macular degeneration (AMD) or retinitis pigmentosa (RP).

73. (canceled)

74. The rAAV particle of claim 1, wherein the heterologous polynucleotide does not encode a fluorescent protein or a sorting or targeting sequence.

75. The rAAV particle of claim 60, wherein the heterologous polynucleotide does not encode a fluorescent protein or a sorting or targeting sequence.

76. The rAAV particle of claim 60, wherein the polynucleotide encoding the channelrhodopsin comprises SEQ ID NO: 17.
